SEQUENCE LISTING (49 sequences)

SEQ ID NO: 1 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 238 aa, Artificial Sequence
  source: /note=“Description of Artificial Sequence: Synthetic polypeptide”
VQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSGGGGSGGGGSIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIK

SEQ ID NO: 3 - protein, 12 aa, Artificial Sequence
  source: /note=“Description of Artificial Sequence: Synthetic peptide”
GGGSGGGSGGGS

SEQ ID NO: 4 - protein, 8 aa, Artificial Sequence
  source: /note=“Description of Artificial Sequence: Synthetic peptide”
YPYDVPDY

SEQ ID NO: 5 - protein, 8 aa, Artificial Sequence
  source: /note=“Description of Artificial Sequence: Synthetic peptide”
DYKDDDDK

SEQ ID NO: 6 - protein, 238 aa, Homo sapiens [sequence omitted]

SEQ ID NO: 7 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 8 - protein, 128 aa, Homo sapiens
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 9 - protein, 348 aa, Erwinia chrysanthemi
MERWFKSLFVLVLFFVFTASAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 10 - protein, 472 aa, Homo sapiens
MELQDPKMNGALPSDAVGYRQEREGFLPSRGPAPGSKPVQFMDFEGKTSFGMSVFNLSNAIMGSGILGLAYAMAHTGVIFFLALLLCIALLSSYSIHLLLTCAGIAGIRAYEQLGQRAFGPAGKVVVATVICLHNVGAMSSYLFIIKSELPLVIGTFLYMDPEGDWFLKGNLLIIIVSVLIILPLALMKHLGYLGYTSGLSLTCMLFFLVSVIYKKFQLGCAIGHNETAMESEALVGLPSQGLNSSCEAQMFTVDSQMSYTVPIMAFAFVCHPEVLPIYTELCRPSKRRMQAVANVSIGAMFCMYGLTATFGYLTFYSSVKAEMLHMYSQKDPLILCVRLAVLLAVTLTVPVVLFPIRRALQQLLFPGKAFSWPRHVAIALILLVLVNVLVICVPTIRDIFGVIGSTSAPSLIFILPSIFYLRIVPSEVEPFLSWPKIQALCFGVLGVLFMAVSLGFMFANWATGQSRMSGH

SEQ ID NO: 11 - protein, 505 aa, Homo sapiens
MKKAEMGRFSISPDEDSSSYSSNSDFNYSYPTKQAALKSHYADVDPENQNFLLESNLGKKKYETEFHPGTTSFGMSVFNLSNAIVGSGILGLSYAMANTGIALFIILLTFVSIFSLYSVHLLLKTANEGGSLLYEQLGYKAFGLVGKLAASGSITMQNIGAMSSYLFIVKYELPLVIQALTNIEDKTGLWYLNGNYLVLLVSLVVILPLSLFRNLGYLGYTSGLSLLCMVFFLIVVICKKFQVPCPVEAALIINETINTTLTQPTALVPALSHNVTENDSCRPHYFIFNSQTVYAVPILIFSFVCHPAVLPIYEELKDRSRRRMMNVSKISFFAMFLMYLLAALFGYLTFYEHVESELLHTYSSILGTDILLLIVRLAVLMAVTLTVPVVIFPIRSSVTHLLCASKDFSWWRHSLITVSILAFTNLLVIFVPTIRDIFGFIGASAASMLIFILPSAFYIKLVKKEPMKSVQKIGALFFLLSGVLVMTGSMALIVLDWVHNPGGGH

SEQ ID NO: 12 - protein, 326 aa, Homo sapiens
TTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMN

SEQ ID NO: 13 - protein, 608 aa, Homo sapiens
ESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNK

SEQ ID NO: 14 - protein, 568 aa, Homo sapiens
ESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRT

SEQ ID NO: 15 - protein, 371 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQP

SEQ ID NO: 16 - protein, 17 aa, Homo sapiens
DYQVSSPIYDINYYTSE

SEQ ID NO: 17 - protein, 29 aa, Homo sapiens
DYQVSSPIYDINYYTSEPCQKINVKQIAA

SEQ ID NO: 18 - protein, 247 aa, Artificial Sequence
  source: /note=“Description of Artificial Sequence: Synthetic polypeptide”
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSGGGGSGGGGSGGGGSDIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIK

SEQ ID NO: 19 - protein, 243 aa, Artificial Sequence
  source: /note=“Description of Artificial Sequence: Synthetic polypeptide”
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 20 - protein, 239 aa, Artificial Sequence
  source: /note=“Description of Artificial Sequence: Synthetic polypeptide”
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSGGGGSGGGGSGGGGSESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 21 - protein, 242 aa, Artificial Sequence
  source: /note=“Description of Artificial Sequence: Synthetic polypeptide”
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKR

SEQ ID NO: 22-31 - skipped (10 entries)

SEQ ID NO: 32 - protein, 5 aa, Unknown
  source: /note=“Description of Unknown: sortase transfer signature peptide”
  MOD_RES (3)..(3): Any amino acid
LPXTG

SEQ ID NO: 33 - protein, 131 aa, Artificial Sequence
  source: /note=“Description of Artificial Sequence: Synthetic polypeptide”
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 34 - protein, 93 aa, Homo sapiens
VSDVPRDLEWAATPTSLLISWDAPAVTVRYYRITYGETGGNSPVQEFTVPGSKSTATISGLKPGVDYTITGYAVTGRGDSPASSKPISINYRT

SEQ ID NO: 35 - protein, 312 aa, Artificial Sequence
  source: /note=“Description of Artificial Sequence: Synthetic polypeptide”
DSFSANQEIRYSEVTPYHVTSVWTKGVTPPAKFTQGEDVFHAPYVANQGWYDITKTFNGKDDLLCGAATAGNMLHWWFDQNKEKIEAYLKKHPDKQKIMFGDQELLDVRKVINTKGDQTNSELFNYFRDKAFPGLSARRIGVMPDLVLDMFINGYYLNVYKTQTTDVNRTYQEKDRRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 36 - protein, 8 aa, Artificial Sequence
  source: /note=“Description of Artificial Sequence: Synthetic peptide”
GGSGGSGG

SEQ ID NO: 37 - protein, 8 aa, Artificial Sequence
  source: /note=“Description of Artificial Sequence: Synthetic peptide”
GGGSGGGS

SEQ ID NO: 38 - protein, 19 aa, Artificial Sequence
  source: /note=“Description of Artificial Sequence: Synthetic peptide”
MYGKIIFVLLLSEIVSISA

SEQ ID NO: 39 - protein, 16 aa, Homo sapiens
CPPCPAPELLGGPSVF

SEQ ID NO: 40 - protein, 15 aa, Homo sapiens
CPPCPAPPVAGPSVF

SEQ ID NO: 41 - protein, 16 aa, Homo sapiens
CPRCPAPELLGGPSVF

SEQ ID NO: 42 - protein, 16 aa, Homo sapiens
AHHAQAPEFLGGPSVF

SEQ ID NO: 43 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 44 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRRGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 45 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRRRSNTLSSPNSKNEKALGIKINSWESSRRGHSFLSNLHLRNGELVIHEKGFYYIYSQTYFRFQEEIKENTKNDKQMVQYIYKYTDYPDPILLMKSARNSCWSKDAEYGLYSIYQGGIFELKENDRIFVSVTNEHLIDMDHEASFFGAFLVG

SEQ ID NO: 46 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 47 - protein, 281 aa, Artificial Sequence
  source: /note=“Description of Artificial Sequence: Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQERIKENTKNDKQMVQYIYKYTSYPDPILLMKSARNSCWSKDAEYGLYSIYQGGIFELKENDRIFVSVTNEHLIDMHHEASFFGAFLVG

SEQ ID NO: 48 - protein, 243 aa, Artificial Sequence
  source: /note=“Description of Artificial Sequence: Synthetic polypeptide”
EVQLVESGGGLVQPGGSLRLSCAASGFNFNDYFMNWVRQAPGKGLEWVAQMRNKNYQYGTYYAESLEGRFTISRDDSKNSLYLQMNSLKTEDTAVYYCARESYYGFTSYWGQGTLVTVSSGGGGSGGGGSGGGGSDIQMTQSPSSLSASVGDRVTITCQASQDIGISLSWYQQKPGKAPKLLIYNANNLADGVPSRFSGSGSGTDFTLTISSLQPEDFATYYCLQHNSAPYTFGQGTKLEIKR

SEQ ID NO: 49 - protein, 243 aa, Artificial Sequence; identical to SEQ ID NO: 19
  source: /note=“Description of Artificial Sequence: Synthetic polypeptide”

(19) United States
(12) Patent Application Publication
(10) Pub. No.: US 2020/0172868 A1
(43) Pub. Date: Jun. 4, 2020

(54) COMPOSITIONS AND METHODS RELATED TO MULTIMODAL THERAPEUTIC CELL SYSTEMS FOR INFECTIOUS DISEASE

(71) Applicant: RUBIUS THERAPEUTICS, INC., Cambridge, MA (US)

(72) Inventors: Tom Wickham, Groton, MA (US); Tiffany F. Chen, Cambridge, MA (US); Nathan Dowden, Winchester, MA (US); Robert Finberg, Northborough, MA (US); Robert J. Deans, Riverside, CA (US); John Round, Cambridge, MA (US); Avak Kahvejian, Lexington, MA (US); Jordi Mata-Fink, Baltimore, MD (US); Noubar B. Afeyan, Lexington, MA (US)

(21) Appl. No.: 16/632,119

(22) PCT Filed: Jul. 19, 2017
(86) PCT No.: PCT/US2017/042868

§ 371 Date: Jan. 17, 2020

(51) Int. Cl. C12N 5/078; C12N 15/86; A61K 35/18; A61P 31/00; A61P 33/06; A61P 35/02

(52) U.S. Cl. CPC C12N 5/0641; C12N 15/86; A61K 35/18; C12N 2740/15043; A61P 33/06; A61P 35/02; C12N 2510/00; A61P 31/00

(57) ABSTRACT

The invention includes compositions and methods related to multimodal therapies, e.g., for treating an infectious disease. A multimodal therapy described herein provides and/or administers a plurality of agents that function in a coordinated manner to provide a therapeutic benefit to a subject in need thereof, e.g., a subject having an infectious disease.

### BACKGROUND

Red blood cells have been considered for use as drug delivery systems, e.g., to degrade toxic metabolites or inactivate xenobiotics, and in other biomedical applications.

### SUMMARY OF THE INVENTION

The invention includes compositions and infectious diseases. A multimodal therapy described herein provides and/or administers a plurality of agents that function in a coordinated manner to provide a therapeutic benefit to a subject in need thereof, e.g., a subject having an infectious disease. In general, a multimodal therapy described herein includes the administration to a subject of a preparation of engineered erythroid cells, e.g., enucleated erythroid cell, comprising (e.g., expressing or containing) a plurality of agents (e.g., polypeptides) that function in a coordinated manner (e.g., agent-additive, agent-synergistic, multiplicative, independent function, localization-based, proximity-dependent, scaffold-based, multimer-based, pathway-based, or compensatory).

In some aspects, the present disclosure provides an enucleated erythroid cell, comprising a first exogenous polypeptide comprising a first infectious disease therapeutic, and a second exogenous polypeptide, comprising a second infectious disease therapeutic.

In some aspects, the present disclosure provides an enucleated erythroid cell, comprising a first exogenous polypeptide comprising a first infectious disease therapeutic, and a second exogenous polypeptide, comprising a second infectious disease therapeutic, wherein the first and second exogenous polypeptides have agent-additive, agent-synergistic, multiplicative, independent function, localization-based, proximity-dependent, scaffold-based, multimer-based, pathway-based, or compensatory activity.

In some aspects, the disclosure provides an enucleated erythroid cell, comprising a first exogenous polypeptide comprising a first infectious disease therapeutic, a second exogenous polypeptide, comprising a second infectious disease therapeutic, and a third exogenous polypeptide comprising a third infectious disease therapeutic.

In some aspects, the disclosure provides an enucleated erythroid cell, comprising a first exogenous polypeptide comprising a first infectious disease therapeutic, and a second exogenous polypeptide, comprising a second infectious disease therapeutic, wherein:
a) the first and second exogenous polypeptides act on the same target, wherein optionally the target is a cell surface receptor and/or an endogenous human protein;
b) the first exogenous polypeptide binds to a first endogenous human protein and the second exogenous polypeptide binds to a second endogenous human target protein, e.g., with a Kd of less than 500, 200, 100, 50, 20, 10, 5, 2, or 1 nM;
c) the first exogenous polypeptide acts on (e.g., binds) a first target, and the second exogenous polypeptide act on (e.g., binds) a second target, wherein the first and second targets are members of the same biological pathway, wherein optionally the targets are cell surface receptors, endogenous human proteins, or both;
d) the first exogenous polypeptide comprises a first pro-apoptotic polypeptide and the second exogenous polypeptide comprises a second pro-apoptotic polypeptide, e.g., a TRAIL receptor ligand, e.g., a TRAIL polypeptide;
e) the first and second exogenous polypeptides are in close proximity to each other, e.g., are less than 10, 7, 5, 4, 3, 2, 1, 0.5, 0.2, or 0.1 nm apart for a duration of at least 1, 2, 5, 10, 30, or 60 seconds; 1, 2, 5, 10, 30, or 60 minutes, or 1, 2, 3, 6, 12, or 14 hours;
f) the first and second exogenous polypeptides have a Kd of less than 500, 200, 100, 50, 20, 10, 5, 2, or 1 nM for each other;
g) the first exogenous polypeptide comprises an antigen-presenting polypeptide, e.g., an MHC molecule, e.g., an MHC class II molecule, and the second exogenous polypeptide comprises an antigen;
h) the first and second exogenous polypeptides act on different targets, wherein optionally at least one of the targets is a cell surface receptor and/or an endogenous human protein, e.g., the first exogenous polypeptide binds a first cell type, e.g., an immune effector cell, and the second exogenous polypeptide binds a second cell type, e.g., an immune effector cell, e.g., a T cell;
i) the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number;
j) the first exogenous polypeptide and the second exogenous polypeptide have a Kd for a first target and a second target, respectively, with a ratio of about 1:1, from about 2:1 to 1:2, from about 5:1 to 1:5, from about 10:1 to 1:10, from about 20:1 to 1:20, from about 50:1 to 1:50, from about 100:1 to 1:100;

k) the first exogenous polypeptide has a first activity (e.g., binding) towards a first target, and the second exogenous polypeptide has a second activity (e.g., binding) towards the first target, e.g., the first and second exogenous polypeptides bind a single target;
l) the first exogenous polypeptide acts on (e.g., binds) a first target and the second exogenous polypeptide acts on (e.g., binds) a second target, and the first and second targets are part of the same pathway, wherein optionally the first exogenous polypeptide acts on the first target and the second exogenous polypeptide acts on the second target simultaneously;
m) the first exogenous polypeptide acts on (e.g., binds) a first target and the second exogenous polypeptide acts on (e.g., binds) a second target, and the first and second targets are part of different pathways, wherein optionally the first and second pathways both act to promote a given cellular response;
n) the first exogenous polypeptide localizes the enucleated erythroid cell to a desired site, e.g., a human cell, and the second exogenous polypeptide has a therapeutic activity, e.g., an immunomodulation activity such as a T cell activation activity or antigen presenting activity;
o) the first exogenous polypeptide binds a first cell, e.g., a first cell type, and the second exogenous polypeptide binds a second cell, e.g., a second cell type, e.g., an immune effector cell, e.g., a T cell;
p) the first exogenous polypeptide and the second exogenous polypeptide are non-human proteins;
q) the first exogenous polypeptide and the second exogenous polypeptide are both enzymes, e.g., biosynthetic enzymes;
r) the first exogenous polypeptide promotes formation of an intermediate molecule and the second exogenous polypeptide acts on the intermediate molecule;
s) the first exogenous polypeptide and the second exogenous polypeptide act on successive steps of a pathway;
t) the erythroid cell comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide; or u) the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide; or
v) the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide.

In some aspects, the disclosure provides an method of treating a subject having an infectious disease (e.g., HIV, Hepatitis B, bacteremia, or LPS toxicity), comprising administering to the subject an effective number of the erythroid cells described herein to the subject, thereby treating the infectious disease.

The disclosure also provides, in some aspects, a method of making an erythroid cell described herein, comprising: a) providing an erythroid cell, b) contacting the erythroid cell with nucleic acid encoding a first exogenous protein (e.g., an exogenous protein of any of Tables 1, 2, 3, or 4) and nucleic acid encoding a second exogenous protein (e.g., an exogenous protein of any of Tables 1, 2, 3, or 4), under conditions that allow uptake of the nucleic acid by the erythroid cell, and c) culturing the cell under conditions that allow for expression of the first and second exogenous proteins, thereby making the erythroid cell.

The disclosure also provides, in some aspects, a plurality of erythroid cells described herein, e.g., wherein the plurality comprises at least 10⁸, 10⁹, 10¹⁰, 10¹¹, or 10¹² erythroid cells according to any of the proceeding claims.

The disclosure also provides, in some aspects, a pharmaceutical composition comprising a cell or plurality of cells described herein.

In some aspects, the present disclosure provides an enucleated erythroid cell, comprising a first exogenous polypeptide comprising a first infectious disease therapeutic, and a second exogenous polypeptide, comprising a second infectious disease therapeutic, wherein the first infectious disease therapeutic is chosen from CD14, TLR2, and TLR4.

In some aspects, the present disclosure provides a method of treating a subject having an infectious disease, comprising administering to the subject an effective number of erythroid cells which comprise a first exogenous polypeptide comprising a first infectious disease therapeutic, and a second exogenous polypeptide, comprising a second infectious disease therapeutic to the subject, wherein the infectious disease is selected from CMV or malaria, thereby treating the infectious disease.

In some aspects, the present disclosure provides a method of treating a subject having an infectious disease, comprising administering to the subject an effective number of erythroid cells which comprise a first exogenous polypeptide comprising a first infectious disease therapeutic, and a second exogenous polypeptide, comprising a second infectious disease therapeutic to the subject, wherein the first infectious disease therapeutic comprises 4-1BB-L, anti PD-L1, ICOS-L, OX40-L, or GITR-L, asparaginase, asparagine transporter SN2, or SAT2, thereby treating the infectious disease.

In some aspects, the present disclosure also provides a method of treating a subject having a cancer, e.g., a leukemia, e.g., AML or ALL, comprising administering to the subject an enucleated erythroid cell comprising a first exogenous polypeptide comprising a tumor starvation enzyme (e.g., asparaginase) and a second exogenous polypeptide comprising a metabolite transporter, e.g., a transporter for a substrate of the tumor starvation enzyme, e.g., SN2 or SAT2.

Any of the aspects herein, e.g., the aspects above, can be characterized by one or more of the embodiments herein, e.g., the embodiments below.

In embodiments, the first exogenous polypeptide or the second exogenous polypeptide comprises: a virus-binding polypeptide, e.g., an antibody molecule, e.g., an scFv; an anti-viral polypeptide, e.g., a protease, e.g., neuraminidase; CD4 or a functional variant or fragment thereof; CCR5 or a functional variant or fragment thereof; TLR4, CD14, MBL, rBPI21, and LPS binding protein, or a functional variant or fragment thereof, or any combination thereof; a viral protein (e.g., Hepatitis B surface antigen or HIV antigen); a vaccine antigen and a polypeptide that induces activation of an APC, e.g. ICOS, CD28, CD40, 4-1BB, or OX40; or an antibacterial protein, e.g., rBPI21.

In embodiments, the first exogenous polypeptide comprises a virus-binding polypeptide, e.g., an antibody molecule, e.g., an scFv, and the second exogenous polypeptide comprises an anti-viral polypeptide, e.g., a protease, e.g., neuraminidase. In embodiments, the first exogenous polypeptide binds a target, e.g., wherein the target comprises a toxin, e.g., anthrax toxin (e.g., PA, EF, or LF, or any combination thereof), a botulinum toxins (A, B, C, D, E, F, G, or any combination thereof), ricin, saxitoxin, Staphyloccocal enterotoxin B, Tetrodotoxin, or Trichothecene mycotoxins. In embodiments, the cell comprises a plurality of antibody molecules, which bind a plurality of common bacterial, fungal, or viral pathogens.

In embodiments, the first exogenous polypeptide binds an antigen presenting cell and the second exogenous polypeptide binds a T cell. In embodiments, the first exogenous polypeptide comprises an MHCII alpha chain and the second exogenous polypeptide comprises an MHCII beta chain polypeptide.

In some embodiments, the first exogenous polypeptide comprises a virus-binding polypeptide, e.g., an antibody molecule, e.g., an scFv, and the second exogenous polypeptide comprises an anti-viral polypeptide, e.g., a protease, e.g., neuraminidase.

In some embodiments,

the first infectious disease therapeutic comprises a CD14 polypeptide and the second infectious disease therapeutic comprises a TLR2 polypeptide, e.g., for the treatment of CMV;

the first infectious disease therapeutic comprises a TLR2 polypeptide and the second infectious disease therapeutic comprises a TLR4 polypeptide, e.g., for the treatment of CMV;

the first infectious disease therapeutic comprises a CD4 polypeptide and the second infectious disease therapeutic comprises a CCR5 polypeptide, e.g., for the treatment of HIV;

the first infectious disease therapeutic comprises a 4-1BB-L polypeptide and the second infectious disease therapeutic comprises an anti PD-L1 polypeptide;

the first infectious disease therapeutic comprises an 1BB-L polypeptide and the second infectious disease therapeutic comprises an ICOS-L polypeptide, an OX40-L polypeptide, or a GITR-L polypeptide; or

the first infectious disease therapeutic comprises an asparaginase polypeptide and the second infectious disease therapeutic comprises an asparagine transporter SN2 or SAT2 polypeptide, e.g., for the treatment of malaria, or any combination thereof.

In some embodiments, the first infectious disease therapeutic comprises a CD4 polypeptide and the second infectious disease therapeutic comprises a CCR5 polypeptide, and at least 80%, 85%, 90%, 95%, 96%, 97%, 98%, or 99% of cells in the population express both the first infectious disease therapeutic and the second infectious disease therapeutic. In some embodiments, the first infectious disease therapeutic comprises a CD4 polypeptide, and at least 80%, 85%, 90%, 95%, 96%, 97%, 98%, or 99% of cells in the population express the first infectious disease therapeutic. In some embodiments, the second infectious disease therapeutic comprises a CCR5 polypeptide, and at least 80%, 85%, 90%, 95%, 96%, 97%, 98%, or 99% of cells in the population express the second infectious disease therapeutic.

In embodiments, the first and second exogenous polypeptides have agent-additive, agent-synergistic, multiplicative, independent function, localization-based, proximity-dependent, scaffold-based, multimer-based, pathway-based, or compensatory activity.

In embodiments, the first exogenous polypeptide binds to a target more strongly than the first exogenous polypeptide binds to the second exogenous polypeptide.

In embodiments, the first exogenous polypeptide promotes fusion of the erythroid cell with a target cell.

In embodiments, e.g., embodiments of methods of making erythroid cells described herein, the nucleic acid encoding the first exogenous protein and the nucleic acid encoding the second exogenous protein are separate nucleic acids. In embodiments, the nucleic acid encoding the first exogenous protein and the nucleic acid encoding the second exogenous protein are part of the same nucleic acid molecule.

In embodiments, the enucleated erythroid cell further comprises a third exogenous polypeptide that comprises a third infectious disease therapeutic.

In embodiments, one or more of (e.g., 2, 3, 4, 5, 10, or more of):
a) the first and second exogenous polypeptides act on the same target, wherein optionally the target is a cell surface receptor, an endogenous human protein, a viral protein, or a bacterial protein;
b) the first exogenous polypeptide binds to a first endogenous human protein and the second exogenous polypeptide binds to a second endogenous human target protein, e.g., with a Kd of less than 500, 200, 100, 50, 20, 10, 5, 2, or 1 nM;
c) the first exogenous polypeptide (e.g., an enzyme) acts on (e.g., binds) a first target, and the second exogenous polypeptide (e.g., an enzyme) act on (e.g., binds) a second target, wherein the first and second targets are members of the same biological pathway, wherein optionally the targets are cell surface receptors, endogenous human proteins (e.g., enzymes), or both;
d) the first and second exogenous polypeptides are in close proximity to each other, e.g., are less than 10, 7, 5, 4, 3, 2, 1, 0.5, 0.2, or 0.1 nm apart for a duration of at least 1, 2, 5, 10, 30, or 60 seconds; 1, 2, 5, 10, 30, or 60 minutes, or 1, 2, 3, 6, 12, or 14 hours; e) the first and second exogenous polypeptides have a Kd of less than 500, 200, 100, 50, 20, 10, 5, 2, or 1 nM for each other;
f) the first and second exogenous polypeptides act on different targets, wherein optionally at least one of the targets is a cell surface receptor and/or an endogenous human protein, e.g., the first exogenous polypeptide binds a first cell type e.g., an immune effector cell, and the second exogenous polypeptide binds a second cell type, e.g., an immune effector cell, e.g., a T cell;
g) the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number;
h) the first exogenous polypeptide and the second exogenous polypeptide have a Kd for a first target and a second target, respectively, with a ratio of about 1:1, from about 2:1 to 1:2, from about 5:1 to 1:5, from about 10:1 to 1:10, from about 20:1 to 1:20, from about 50:1 to 1:50, from about 100:1 to 1:100;
i) the first exogenous polypeptide has a first activity (e.g., binding) towards a first target, and the second exogenous polypeptide has a second activity (e.g., binding) towards the first target, e.g., the first and second exogenous polypeptides bind a single target;
j) the first exogenous polypeptide acts on (e.g., binds) a first target and the second exogenous polypeptide acts on (e.g., binds) a second target, and the first and second targets are part of the same pathway, wherein optionally the first exogenous polypeptide acts on the first target and the second exogenous polypeptide acts on the second target simultaneously;
k) the first exogenous polypeptide acts on (e.g., binds) a first target and the second exogenous polypeptide acts on (e.g., binds) a second target, and the first and second targets are part of different pathways, wherein optionally the first and second pathways both act to promote a given cellular response;
l) the first exogenous polypeptide localizes the enucleated erythroid cell to a desired site, e.g., infected tissue, and the second exogenous polypeptide has a therapeutic activity, e.g., antibacterial or antiviral activity;
m) the first exogenous polypeptide binds a first cell, e.g., a first cell type, and the second exogenous polypeptide binds a second cell, e.g., a second cell type, e.g., an immune effector cell, e.g., a T cell;
n) the first exogenous polypeptide and the second exogenous polypeptide are non-human proteins, e.g., the enzymes are not natively found in humans;
o) the first exogenous polypeptide and the second exogenous polypeptide are both enzymes, e.g., biosynthetic enzymes;
p) the first exogenous polypeptide (e.g., an enzyme) promotes formation of an intermediate molecule (e.g., converts a substrate into an intermediate) and the second exogenous polypeptide (e.g., an enzyme) acts on the intermediate molecule (e.g., converts an intermediate into a product);
q) the first exogenous polypeptide and the second exogenous polypeptide act on successive steps of a pathway;
r) the erythroid cell comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide;
s) the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide; or
t) the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide.

In embodiments, at least:

(a) and (b), (a) and (c), (a) and (d), (a) and (e), (a) and (f), (a) and (g), (a) and (h), (a) and (i), (a) and (j), (a) and (k), (a) and (l), (a) and (m), (a) and (n), (a) and (o), (a) and (p), (a) and (q), (a) and (r), (a) and (s), (a) and (t),

(b) and (c), (b) and (d), (b) and (e), (b) and (f), (b) and (g), (b) and (h), (b) and (i), (b) and (j), (b) and (k), (b) and (l), (b) and (m), (b) and (n), (b) and (o), (b) and (p), (b) and (q), (b) and (r), (b) and (s), (b) and (t),

(c) and (d), (c) and (e), (c) and (f), (c) and (g), (c) and (h), (c) and (i), (c) and (j), (c) and (k), (c) and (l), (c) and (m), (c) and (n), (c) and (o), (c) and (p), (c) and (q), (c) and (r), (c) and (s), (c) and (t),

(d) and (e), (d) and (f), (d) and (g), (d) and (h), (d) and (i), (d) and (j), (d) and (k), (d) and (l), (d) and (m), (d) and (n), (d) and (o), (d) and (p), (d) and (q), (d) and (r), (d) and (s), (d) and (t),

(e) and (f), (e) and (g), (e) and (h), (e) and (i), (e) and (j), (e) and (k), (e) and (l), (e) and (m), (e) and (n), (e) and (o), (e) and (p), (e) and (q), (e) and (r), (e) and (s), (e) and (t),

(f) and (g), (f) and (h), (f) and (i), (f) and (j), (f) and (k), (f) and (l), (f) and (m), (f) and (n), (f) and (o), (f) and (p), (f) and (q), (f) and (r), (f) and (s), (f) and (t),

(g) and (h), (g) and (i), (g) and (j), (g) and (k), (g) and (l), (g) and (m), (g) and (n), (g) and (o), (g) and (p), (g) and (q), (g) and (r), (g) and (s), (g) and (t),

(h) and (i), (h) and (j), (h) and (k), (h) and (l), (h) and (m), (h) and (n), (h) and (o), (h) and (p), (h) and (q), (h) and (r), (h) and (s), (h) and (t),

(i) and (j), (i) and (k), (i) and (l), (i) and (m), (i) and (n), (i) and (o), (i) and (p), (i) and (q), (i) and (r), (i) and (s), (i) and (t),

(j) and (k), (j) and (l), (j) and (m), (j) and (n), (j) and (o), (j) and (p), (j) and (q), (j) and (r), (j) and (s), (j) and (t),

(k) and (l), (k) and (m), (k) and (n), (k) and (o), (k) and (p), (k) and (q), (k) and (r), (k) and (s), (k) and (t),

(l) and (m), (l) and (n), (l) and (o), (l) and (p), (l) and (q), (l) and (r), (l) and (s), (l) and (t),

(m) and (n), (m) and (o), (m) and (p), (m) and (q), (m) and (r), (m) and (s), (m) and (t),

(n) and (o), (n) and (p), (n) and (q), (n) and (r), (n) and (s), (n) and (t),

(o) and (p), (o) and (q), (o) and (r), (o) and (s), (o) and (t),

(p) and (q), (p) and (r), (p) and (s), (p) and (t),

(q) and (r), (q) and (s), (q) and (t),

(r) and (s), (r) and (t), or

(s) and (t), are present.

In some embodiments, the exogenous polypeptides have synergistic activity. In some embodiments, the exogenous polypeptides have additive activity. In some embodiments, the exogenous polypeptides have proximity-dependent activity. The proximity between the plurality of polypeptides, before, during, or after, interaction with a target moiety or moieties, may confer a property or result which is not seen in the absence of such proximity in vivo or in vitro.

In some embodiments, the first exogenous polypeptide interacts with, e.g., binds, a first target moiety, e.g., a first target cell polypeptide on a target cell (e.g., an immune effector cell, e.g., a T cell), and the second exogenous polypeptide interacts with, e.g., binds, a second target moiety, e.g., a second target cell polypeptide on the target cell (e.g., wherein binding of the first and second target cell polypeptide alters a biological property of the target cell). In an embodiment the first and second targets are subunits of a multimeric complex on the target cell.

In some embodiments, the first exogenous polypeptide promotes fusion of the enucleated erythroid cell with a target cell and the second exogenous polypeptide is a polypeptide of any of Table 1, Table 2, Table 3, or Table 4 (e.g., a human polypeptide of any of Table 1, Table 2, Table 3, or Table 4, e.g., a polypeptide having the amino acid sequence of the human wild type polypeptide).

In some embodiments the first and second exogenous polypeptides interact with one another, e.g., the first modifies, e.g., by cleavage or phosphorylation, the second, or the first and second form a dimeric or multimeric protein.

In some embodiments, the enucleated erythroid cell comprises 3, 4, 5, 6, 7, 8, 9, or 10 different exogenous polypeptides. In an embodiment a plurality (e.g., 2, 3, 4, 5, 6, 7, 8, 9, or 10), or all, of the different exogenous polypeptides, have a preselected level of homology to each other, e.g., at least 40, 50, 60, 70, 75, 80, 85, 90, 95, 96, 97, 98, 99, or 99.5% sequence identity to each other. In an embodiment a plurality (e.g., 2, 3, 4, 5, 6, 7, 8, 9, or 10), or all, of the different exogenous polypeptides, have a preselected level of homology to a reference sequence, e.g., at least 40, 50, 60, 70, 75, 80, 85, 90, 95, 96, 97, 98, 99, 99.5%, or 100% sequence identity with a reference sequence (which reference sequence, includes an entire polypeptide sequence, or a portion thereof, e.g., a preselected domain), e.g., a plurality or all of the different exogenous polypeptides are antibodies or antibody molecules. In some embodiments, the reference sequence is an antibody sequence or fragment thereof. In some embodiments, the reference sequence comprises a heavy chain constant region or portion thereof, light chain constant region or fragment thereof, heavy chain variable region or portion thereof, light chain variable region or fragment thereof, or any combination of the foregoing. In embodiments, the enucleated erythroid cells are used to stimulate an immune response in a subject having an infectious disease, e.g., as a substitute for (or combination with) an IVIG therapy.

In some embodiments, the enucleated erythroid cell comprises at least 2 but no more than 5, 6, 7, 8, 9, or 10 different exogenous polypeptides, e.g., exogenous polypeptides that are encoded by one or more exogenous nucleic acids that are not retained by the enucleated erythroid cell.

In some embodiments, the exogenous polypeptides are encoded by one or more exogenous nucleic acids that are not retained by the enucleated erythroid cell.

In some embodiments, one or more (e.g., two or three) of the first, second, and optionally third exogenous polypeptides are transmembrane polypeptides or surface-anchored polypeptides. In some embodiments, the first exogenous polypeptide is a transmembrane polypeptides or surface-anchored polypeptide, and the second exogenous polypeptide is internal to the erythroid cell and not associated with the cell membrane. In some embodiments, the second exogenous polypeptide is a transmembrane polypeptides or surface-anchored polypeptide, and the first exogenous polypeptide is internal to the erythroid cell and not associated with the cell membrane. In some embodiments, both of the first and second exogenous polypeptides are internal to the erythroid cell and not associated with the cell membrane.

In some embodiments, the first exogenous polypeptide interacts with, e.g., binds, a moiety on a target cell, and the second exogenous polypeptide alters a property of the target cell, e.g., kills or activates the target cell.

In some embodiments, the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, or from about 100:1 to 1:100 by weight or by copy number. In some embodiments, both the first and second polypeptides have a stoichiometric mode of action, or both have a catalytic mode of action, and both are present at a similar abundance, e.g., about 1:1 or from about 2:1 to 1:2. In some embodiments, the first exogenous polypeptide is more abundant than the second exogenous polypeptide by at least about 10%, 20%, 30%, 50%, or a factor of 2, 3, 4, 5, 10, 20, 50, or 100 (and optionally up to 10 or 100 fold) by weight or copy number. In some embodiments, the second exogenous polypeptide is more abundant than the first exogenous polypeptide by at least about 10%, 20%, 30%, 50%, or a factor of 2, 3, 4, 5, 10, 20, 50, or 100 (and optionally up to 10 or 100 fold) by weight or copy number. In some embodiments, the first polypeptide has a stoichiometric mode of action and the second polypeptide has a catalytic mode of action, and the first polypeptide is more abundant than the second polypeptide. In some embodiments, the second polypeptide has a stoichiometric mode of action and the first polypeptide has a catalytic mode of action, and the second polypeptide is more abundant than the first polypeptide.

In some embodiments, the first exogenous polypeptide comprises a targeting moiety.

In some embodiments, the enucleated erythroid cell has one or more of the following characteristics:
a) an osmotic fragility of less than 50% cell lysis at 0.3%, 0.35%, 0.4%, 0.45%, or 0.5%

NaCl;
b) a cell volume of about 10-200 fL or a cell diameter of between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns, or between about 10 microns and about 30 microns;
c) greater than 1%, 2%, 3%, 4%, 5%, 6%, 7%, 8%, 9% or 10% fetal hemoglobin; or at least about 20, 25, or 30 pg/cell of hemoglobin; or
d) phosphatidylserine content of the outer leaflet is less than 30%, 25%, 20%, 15%, 10%, or 5% as measured by Annexin V staining.

In some embodiments, at least one, e.g., all, of the plurality of exogenous polypeptides are glycosylated. In some embodiments, at least one, e.g., all, of the plurality of exogenous polypeptides are phosphorylated.

In some embodiments, the enucleated erythroid cell is a reticulocyte.

In some embodiments, the exogenous polypeptide or polypeptides lack a sortase transfer signature (i.e., a sequence that can be created by a sortase reaction) such as LPXTG (SEQ ID NO: 32).

In some embodiments, the enucleated erythroid cell comprises: a first exogenous polypeptide that interacts with a target, and a second exogenous polypeptide that modifies the target.

In some embodiments, one or more of:

(a) the second exogenous polypeptide comprises an enzyme (e.g., a protease) that modifies, e.g., is specific, e.g., binds to a site on target, binds (e.g., specifically) and modifies, e.g., covalently modifies, e.g., cleaves, or removes or attaches a moiety to, the target, wherein the target is optionally a viral factor, e.g., a viral protein, or a bacterial factor, e.g., a bacterial protein;

(b) the second exogenous polypeptide comprises a polypeptide, e.g., an enzyme, e.g., a protease, that modifies the secondary, tertiary, or quaternary structure of the target, and, in embodiments, alters, e.g., decreases or increases, the ability of the target to interact with another molecule, e.g., the first exogenous polypeptide or a molecule other than the first exogenous polypeptide, wherein optionally the target comprises a viral factor or a bacterial factor;

(c) the second exogenous polypeptide comprises a polypeptide, e.g., an enzyme (e.g., a protease) that cleaves the target, e.g., a polypeptide, between a first target domain and a second target domain, e.g., a first target domain that binds a first substrate and a second target domain that binds a second substrate;

(d) the target is a polypeptide (e.g., a viral factor or bacterial factor); a carbohydrate (e.g., a glycan), a lipid (e.g., a phospholipid), or a nucleic acid (e.g., DNA, or RNA);

(e) the first exogenous polypeptide binds but does not cleave a target and the second exogenous polypeptide cleaves a bond e.g., a covalent bond, e.g., a covalent bond in the target;

(f) the first exogenous polypeptide has an affinity for the target that is about 1-2 pM, 2-5 pM, 5-10 pM, 10-20 pM, 20-50 pM, 50-100 pM, 100-200 pM, 200-500 pM, 500-1000 pM, 1-2 nM, 2-5 nM, 5-10 nM, 10-20 nM, 20-50 nM, 50-100 nM, 100-200 nM, 200-500 nM, 500-1000 nM, 1-2 μM, 2-5 μM, 5-10 μM, 10-20 μM, 20-50 μM, or 50-100 μM;

(g) the second exogenous polypeptide has a K_{M} for the target of about 10⁻¹-10⁻⁷M, 10⁻¹ -10⁻²M, 10⁻²-10⁻³M, 10⁻³-10⁻⁴M, 10⁻⁴-10⁻⁵M, 10⁻⁵-10⁻⁶M, or 10⁻⁶-10⁻⁷M;

(h) a ratio of the K_{d} of the first exogenous polypeptide for the target (measured in M) divided by the K_{M} of the second exogenous polypeptide for the target (measured in M) is about 1×10⁻⁹-2×10⁻⁹, 2×10⁻⁹-5×10⁻⁹, 5×10⁻⁹-1×10⁻⁸, 1×10⁻⁸-2×10⁻⁸, 2×10⁻⁸-5×10⁻⁸, 5×10⁻⁸-1×10⁻⁷, 1×10⁻⁷-2×10⁻⁷, 2×10⁻⁷-5×10⁻⁷, 5×10⁻⁷-1×10⁻⁶, 1×10⁻⁶-2×10⁻⁶, 2×10⁻⁶-5×10⁻⁶, 5×10⁻⁶-1×10⁻⁵, 1×10⁻⁵-2×10⁻⁵, 2×10⁻⁵-5×10⁻⁵, 5×10⁻⁵-1×10⁻⁴, 1×10-2×10⁻⁴, 2×10-5×10⁻⁴, 5×10⁻⁴-1×10³, 1×10³-2×10³, 2×10³-5×10³, 5×10³-1×10², 1×10²-2×10², 2×10⁻²-5×10⁻², 5×10⁻²-1×10⁻¹, 1×10⁻¹-2×10⁻¹, 2×10⁻¹-5×10⁻¹, or 5×10⁻¹-1;

(i) the observed reaction rate of the second exogenous polypeptide modifying the target is greater than the reaction rate of an enucleated cell which is similar but which lacks the first exogenous polypeptide under otherwise similar reaction conditions;

(j) a ratio of the average number of the first exogenous polypeptide on the erythroid cell to the average number of the second exogenous polypeptide on the erythroid cell is about 50:1, 20:1, 10:1, 8:1, 6:1, 4:1, 2:1, 1:1, 1:2, 1:4, 1:6, 1:8, 1:10, 1:20, or 1:50;

(k) affinity of the first exogenous polypeptide for the target is greater than the affinity of the first exogenous polypeptide for the modified (e.g., cleaved) target;

(l) a therapeutically effective dose of the enucleated erythroid cell is less than stoichiometry (e.g., less by 10%, 20%, 30%, 40%, 50%, 60%, 70%, 80%, 90%, 95%, 98%, 99%, 99.5%, 99.9%, or 99.99%) to the amount of target in a subject's peripheral blood at the time of administration;

(m) the number of enucleated erythroid cells in an effective dose, is less than (e.g., less by 10%, 20%, 30%, 40%, 50%, 60%, 70%, 80%, 90%, 95%, 98%, 99%, 99.5%, 99.9%, or 99.99%) the number of targets, e.g., target molecules, in the subject's peripheral blood at the time of administration;

(n) the number of second exogenous polypeptides comprised by a preselected amount of enucleated erythroid cells, e.g., an effective dose, or in vitro effective amount of enucleated erythroid cells, is less than (e.g., less by 10%, 20%, 30%, 40%, 50%, 60%, 70%, 80%, 90%, 95%, 98%, 99%, 99.5%, 99.9%, or 99.99%) a reference value for targets, e.g., less than the number of targets in the peripheral blood of the subject at the time of administration;

(o) the number of first exogenous polypeptides comprised by a preselected amount of enucleated erythroid cells, e.g., an effective dose, or in vitro effective amount of enucleated erythroid cells, is less than (e.g., less by 10%, 20%, 30%, 40%, 50%, 60%, 70%, 80%, 90%, 95%, 98%, 99%, 99.5%, 99.9%, or 99.99%) a reference value for targets, e.g., less than the number of targets in the peripheral blood of the subject at the time of administration;

(p) the number of first exogenous polypeptides and the number of second exogenous polypeptides comprised by a preselected amount of enucleated erythroid cells, e.g., an effective dose, enucleated erythroid cells, is each less than a reference value for targets, e.g., less than the number of targets in the peripheral blood of the subject at the time of administration;

(q) the second exogenous polypeptide modifies (e.g., cleaves) the target with a K_{M} of at least 10⁻¹ M, 10⁻²M, 10⁻³ M, 10⁻⁴ M, 10⁻⁵ M, 10⁻⁶M, or 10⁻⁷ M;

(r) the second exogenous polypeptide comprises a chaperone;

(s) the first exogenous polypeptide comprises a surface-exposed portion and the second exogenous polypeptide comprises a surface exposed portion; or

(t) an effective amount of the enucleated erythroid cells is less than (e.g., less by 10%, 20%, 30%, 40%, 50%, 60%, 70%, 80%, 90%, 95%, 98%, 99%, 99.5%, 99.9%, or 99.99%) an effective amount of otherwise similar enucleated erythroid cells that lack the second exogenous polypeptide.

In embodiments, (a) the second exogenous polypeptide comprises an enzyme (e.g., a protease) that modifies, e.g., is specific, e.g., binds to a site on target, binds (e.g., specifically) and modifies, e.g., covalently modifies, e.g., cleaves, or removes or attaches a moiety to, the target, wherein the target is optionally a viral factor or bacterial factor. In embodiments the modification alters, e.g., increases or decreases, the ability of the target to interact with another molecule, e.g., the first exogenous polypeptide or a molecule other than the first exogenous polypeptide.

In embodiments, (c) the second exogenous polypeptide comprises a polypeptide, e.g., an enzyme (e.g., a protease) that cleaves the target, e.g., a polypeptide, between a first target domain and a second target domain, e.g., a first target domain that binds a first substrate and a second target domain that binds a second substrate. In embodiments the first target domain is released from the second target domain. In embodiments cleavage alters the affinity one or both of the first target domain for a first substrate and the affinity of the second target domain for a second substrate.

In embodiments, the target is other than an infectious component, e.g., other than a bacterial component, a viral component, a fungal component, or a parasitic component. In embodiments, the surface-exposed portion of the first exogenous polypeptide binds the target. In embodiments, the surface-exposed portion of the second exogenous polypeptide comprises enzymatic activity, e.g., protease activity. In embodiments, the surface-exposed portion of the second exogenous polypeptide enzymatically modifies, e.g., cleaves, the target. In embodiments, the enucleated erythroid cell is capable of clearing the target from a subject's body at a faster rate than an otherwise similar enucleated erythroid cell that lacks the second exogenous polypeptide. In embodiments, the enucleated erythroid cell is complexed with the target or a reaction product of the second exogenous protein acting on the target, e.g., during cleavage.

In some embodiments of any of the compositions and methods described herein,

i) at least 50, 60, 70, 80, 90, 95, or 99% of the exogenous polypeptides, e.g., fusion proteins on the surface of the erythroid cell have an identical sequence,

ii) at least 50, 60, 70, 80, 90, 95, or 99% of the exogenous polypeptides, e.g., fusion proteins have the same transmembrane region,

iii) the first and/or second exogenous polypeptide, e.g., fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;

iv) at least 50, 60, 70, 80, 90, 95, or 99% of the exogenous polypeptides, e.g., fusion proteins do not differ from one another by more than 1, 2, 3, 4, 5, 10, 20, or 50 amino acids,

v) the first and/or second exogenous polypeptide lacks a sortase transfer signature,

vi) the first and/or second exogenous polypeptide comprises a moiety that is present on less than 1, 2, 3, 4, or 5 sequence distinct fusion polypeptides;

vii) the first and/or second exogenous polypeptide is present as a single fusion polypeptide;

viii) the first and/or second exogenous polypeptide, e.g., fusion protein does not contain Gly-Gly at the junction of an endogenous transmembrane protein and the moiety;

ix) the first and/or second exogenous polypeptide, e.g., fusion protein does not contain

Gly-Gly, or the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof.

In some aspects, the present disclosure provides a method of treating a disease or condition described herein, comprising administering to a subject in need thereof an enucleated erythroid cell described herein. In some embodiments, the disease or condition is an infectious disease.

In some aspects, the present disclosure provides a method of bringing into proximity a first and a second cell surface moiety, e.g., transmembrane receptors, e.g., receptors of an infected cell, comprising administering to a subject in need thereof an enucleated erythroid cell described herein.

In some aspects, the present disclosure provides a method of delivering, presenting, or expressing a plurality of proximity-dependent molecules comprising providing an enucleated erythroid cell described herein.

In some aspects, the present disclosure provides a method of producing an enucleated erythroid cell described herein, providing contacting an erythroid cell precursor with one or more nucleic acids encoding the exogenous polypeptides and placing the cell in conditions that allow enucleation to occur.

In some aspects, the present disclosure provides a preparation, e.g., pharmaceutical preparation, comprising a plurality of enucleated erythroid cells described herein, e.g., at least 10⁸, 10⁹, 10¹⁰, 10¹¹, or 10¹² cells.

In some aspects, the present disclosure provides a cell complex, e.g., an in vitro or in vivo complex, of an engineered erythroid cell, e.g., an enucleated erythroid cell, and a target cell, the complex mediated by one of the exogenous polypeptides. In some embodiments, the cell complex comprises at least 2, 3, 4, 5, 10, 20, 50, or 100 cells.

In some aspects, the present disclosure proves a reaction mixture comprising an engineered erythroid cell, e.g., an enucleated erythroid cell, and nucleic acid, e.g., one or more nucleic acid molecules, encoding a multimodal pair described herein. In some embodiments, the nucleic acid comprises at least one promoter that is active in an erythroid cell. In some embodiments, nucleic acid encodes at least two proteins described herein (e.g., in Table 1, Table 2, Table 3, and Table 4). In some embodiments, the nucleic acid encodes a third exogenous polypeptide.

In some aspects, the present disclosure comprises a method of making an engineered erythroid cell (e.g., an enucleated erythroid cell) described herein, comprising: providing, e.g., receiving, information about a target cell or subject, responsive to that information selecting a plurality of exogenous polypeptides, and introducing nucleic acids encoding the exogenous polypeptides into an erythroid cell or erythroid cell precursor.

In some aspects, the present invention comprises a method of evaluating an engineered erythroid cell (e.g., enucleated erythroid cell), comprising providing a candidate erythroid cell, and determining if nucleic acid encoding a plurality of exogenous polypeptides, e.g., a multimodal pair of the exogenous polypeptides, are present.

The present disclosure also provides, in some aspects, a nucleic acid composition comprising:

a first nucleic acid sequence encoding a first exogenous polypeptide that interacts with a target, e.g., a first exogenous polypeptide described herein,

a second nucleic acid sequence encoding a second exogenous polypeptide that modifies the target, e.g., a second nucleic acid sequence described herein and

optionally, a promoter sequence that is active in an erythroid cell.

In embodiments, the first nucleic acid sequence and second nucleic acid sequence are contiguous or are separate molecules (e.g., admixed molecules or in separate containers). In embodiments, the first nucleic acid sequence and second nucleic acid sequence are part of the same open reading frame and have a protease cleavage site situated therebetween. In embodiments, the first nucleic acid is operatively linked to a first promoter and the second nucleic acid is operatively linked to a second promoter.

The disclosure provides, in some aspects, a kit comprising:

(A) nucleic acids encoding: (A-i) a plurality of binding moieties (e.g., antibody molecules, e.g., scFv domains), fused to (A-ii) a membrane anchor domain, e.g., a transmembrane domain, wherein (A-i) and (A-ii) are operatively linked to a nucleic acid that directs expression in an erythroid cell; and

(B) nucleic acids encoding (B-i) a plurality of enzymes (e.g., proteases), optionally fused to (B-ii) a membrane anchor domain, e.g., a transmembrane domain, wherein (B-i) and (B-ii) are operatively linked to nucleic acid that directs expression in an erythroid cell.

The present disclosure provides, in some aspects, a method of making a fragment of a target, e.g., a bacterial factor or viral factor, e.g., a viral capsid protein, comprising contacting the target polypeptide with an erythroid cell described herein. In embodiments, the second exogenous polypeptide cleaves the target to provide the fragment. In embodiments, the contacting comprises administering the erythroid cell to a subject that comprises the target polypeptide. In embodiments, the fragment inhibits the corresponding full-length protein, e.g., a fragment of a viral capsid protein inhibits assembly of the viral capsid.

The present disclosure also provides, in some aspects, a method of converting or activating a target, e.g., a bacterial factor or viral factor, e.g., converting a prodrug to a drug, comprising contacting the polypeptide with an erythroid cell described herein. In embodiments, the second exogenous polypeptide interacts with the target (e.g., prodrug), e.g., cleaves the target. In embodiments, the contacting comprises administering the erythroid cell to a subject that comprises the polypeptide, e.g., prodrug.

The present disclosure also provides, in some aspects, a method of converting an endogenous polypeptide from a first activity state to a second activity state (e.g., from an inactive state to an active state or an active state to an inactive state), comprising contacting the endogenous polypeptide with an erythroid cell described herein. In embodiments, the second exogenous polypeptide interacts with the target, e.g., covalently modifies, e.g., cleaves the target, or alters its ability to interact with, e.g., bind, another molecule. In embodiments, the contacting comprises administering the erythroid cell to a subject that comprises the endogenous polypeptide.

The disclosure provides, in some aspects, a method of reducing a level of a target (e.g., a viral factor or bacterial factor) in a subject, comprising administering to the subject an erythroid cell described herein. In embodiments, the second exogenous polypeptide interacts with the target, e.g., covalently modifies, e.g., cleaves the target, or alters its ability to interact with, e.g., bind, another molecule.

The disclosure contemplates all combinations of any one or more of the foregoing aspects and/or embodiments, as well as combinations with any one or more of the embodiments set forth in the detailed description and examples.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Jul. 19, 2017. When one gene or protein references a plurality of sequence accession numbers, all of the sequence variants are encompassed.

### BRIEF DESCRIPTION OF THE DRAWINGS

FIG. 1 is a set of graphs showing results of a Raji apoptosis assay measured through flow cytometry. Raji cells are CFSE labeled and co-cultured with erythroid differentiated cells that are untransduced (control) and transduced with single or multiple TRAIL variants or co-cultured with two different singly transduced cells. Percent apoptosis determined by percent of cells that are Raji (CFSE+) and annexin V+. (Top) Flow cytometry plots of CFSE and annexin V staining of various conditions. (Bottom) Graph of percent apoptosis of the various conditions.

FIG. 2 is a set of graphs showing expression of 4-1BB-L and anti-PD-L1 from enucleated erythroid cells comprising 4-1BB-L and anti-PD-L1 as assessed using flow cytometry.

FIG. 3 is a set of graphs showing IFNg, IL-2 and TNFa secretion in peripheral blood mononuclear cells (PBMC) via ELISA following addition of enucleated erythroid cells comprising 4-1BB-L, anti-PD-L1, or 4-1BB-L and anti-PD-L1 in combination. Controls include PBMC and HA-GPA enucleated erythroid cells.

FIG. 4 is a set of graphs showing expression of 4-1BB-L, ICOS-L, and 4-1BB-L and ICOS-L from enucleated erythroid cells as assessed using flow cytometry.

FIG. 5 is a set of graphs showing IFNg and IL-2 secretion in PBMC as assessed by ELISA following addition of enucleated erythroid cells comprising 4-1BB-L, ICOS-L, or 4-1BB-Land ICOS-L in combination. Controls include PBMC, control erythroid cells (UNT), and HA-GPA enucleated erythroid cells.

FIG. 6 is a set of graphs showing proliferation of PBMC as measured by the total number of CD4 and CD8 cells following co-culture with enucleated erythroid cells expressing 4-1BB-L, ICOS-L, OX40-L, GITR-L, and combinations of 4-1BB-L/ICOS-L and 4-1BB-L/GITR-L.

FIG. 7 is a set of graphs showing proliferation of PBMC as measured by the number of proliferating CD4 and CD8 cells following co-culture with enucleated erythroid cells expressing 4-1BB-L, ICOS-L, OX40-L, GITR-L, and combinations of 4-1BB-L/ICOS-L and 4-1BB-L/GITR-L. Controls include PBMC stimulated with CD3 and without CD3, and CDS including HA-GPA.

FIG. 8 is a bar graph showing the mean fluorescent intensity from control erythroid cells (UNT) or IdeS-expressing erythroid cells (IDES) labelled with an anti-Rabbit Fc fluorophore labeled antibody, before or after a 5 hour incubation.

FIG. 9 is a Western blot showing intact heavy chain of target antibodies or fragments of the heavy chain in supernatant from control cells (UNT) or Ide-S expressing cells (IdeS-RCT). Arrows indicate the heavy chain (Hc), heavy chain fragment (Hc-fragment), and light chain (Lc).

### DETAILED DESCRIPTION OF THE INVENTION

### Definitions

As used herein, the term “antibody molecule” refers to a protein, e.g., an immunoglobulin chain or fragment thereof, comprising at least one immunoglobulin variable domain sequence. The term “antibody molecule” encompasses antibodies and antibody fragments. In an embodiment, an antibody molecule is a multispecific antibody molecule, e.g., a bispecific antibody molecule. Examples of antibody molecules include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, an isolated epitope binding fragment of an antibody, maxibodies, minibodies, nanobodies, intrabodies, diabodies, triabodies, tetrabodies, v-NAR and bis-scFv.

As used herein, a “combination therapy” or “administered in combination” means that two (or more) different agents or treatments are administered to a subject as part of a treatment regimen for a particular disease or condition. The treatment regimen includes the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

The term “coordinated” or “coordinated manner” means that a plurality of agents work together to provide a therapeutic benefit. Types of coordinated activity include agent-additive, agent-synergistic, multiplicative, independent function, localization-based, proximity-dependent, scaffold-based, multimer-based, pathway-based, and compensatory activity. In an embodiment the level of therapeutic benefit conferred by a plurality of exogenous polypeptides delivered in the same enucleated erythroid cell is greater than would be seen if each of the plurality of polypeptides were delivered from different enucleated erythroid cells.

As used herein, “enucleated” refers to a cell, e.g., a reticulocyte or mature red blood cell that lacks a nucleus. In an embodiment an enucleated cell is a cell that has lost its nucleus through differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell, into a reticulocyte or mature red blood cell. In an embodiment an enucleated cell is a cell that has lost its nucleus through in vitro differentiation from a precursor cell, e.g., a hematopoietic stem cell (e.g., a CD34+ cell), a common myeloid progenitor (CMP), a megakaryocyte erythrocyte progenitor cell (MEP), a burst-forming unit erythrocyte (BFU-E), a colony-forming unit erythrocyte (CFU-E), a pro-erythroblast, an early basophilic erythroblast, a late basophilic erythroblast, a polychromatic erythroblast, or an orthochromatic erythroblast, or an induced pluripotent cell into a reticulocyte or mature red blood cell.

“Erythroid cell” as used herein, includes a nucleated red blood cell, a red blood cell precursor, an enucleated mature red blood cell, and a reticulocyte. For example, any of a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, is an erythroid cell. A preparation of erythroid cells can include any of these cells or a combination thereof. In some embodiments, the erythroid cells are immortal or immortalized cells. For example, immortalized erythroblast cells can be generated by retroviral transduction of CD34+hematopoietic progenitor cells to express Oct4, Sox2, Klf4, cMyc, and suppress TP53 (e.g., as described in Huang et al., Mol. Ther. 2013, epub ahead of print September 3). In addition, the cells may be intended for autologous use or provide a source for allogeneic transfusion. In some embodiments, erythroid cells are cultured. In an embodiment an erythroid cell is an enucleated red blood cell.

As used herein, the term “exogenous polypeptide” refers to a polypeptide that is not produced by a wild-type cell of that type or is present at a lower level in a wild-type cell than in a cell containing the exogenous polypeptide. In some embodiments, an exogenous polypeptide is a polypeptide encoded by a nucleic acid that was introduced into the cell, which nucleic acid is optionally not retained by the cell. In some embodiments, an exogenous polypeptide is a polypeptide conjugated to the surface of the cell by chemical or enzymatic means.

“Infectious disease therapeutic” as used herein, refers to an exogenous polypeptide which inhibits an infectious disease, e.g., reduces or alleviates a cause or symptom of an infectious disease, or improves a value for a parameter associated with the infectious disease, e.g., viral load or bacterial load. In embodiments, the infectious disease therapeutic is a first or second exogenous polypeptide, which when present or expressed with the other exogenous polypeptide, inhibits an infectious disease. In an embodiment, a first or second infectious disease therapeutic has activity in the absence of the other. In embodiments, the infectious disease therapeutic inhibits the infectious disease directly, e.g., by killing pathogens. In embodiments, the infectious disease therapeutic inhibits the infectious disease by stimulating a subject's immune response, e.g., as a vaccine.

As used herein, the term “multimodal therapy” refers to a therapy, e.g., an enucleated erythroid cell therapy, that provides a plurality (e.g., 2, 3, 4, or 5 or more) of exogenous agents (e.g., polypeptides) that have a coordinated function (e.g., agent-additive, agent-synergistic, multiplicative, independent function, localization-based, proximity-dependent, scaffold-based, multimer-based, pathway-based, or compensatory activity).

As used herein, the term “pathway” or “biological pathway” refers to a plurality of biological molecules, e.g., polypeptides, that act together in a sequential manner. Examples of pathways include signal transduction cascades and complement cascades. In some embodiments, a pathway begins with detection of an extracellular signal and ends with a change in transcription of a target gene. In some embodiments, a pathway begins with detection of a cytoplasmic signal and ends with a change in transcription of a target gene. A pathway can be linear or branched. If branched, it can have a plurality of inputs (converging), or a plurality of outputs (diverging).

As used herein, a “proximity-dependent” molecule refers to a first molecule that has a different, e.g., greater, activity when in proximity with a second molecule than when alone. In some embodiments, a pair of proximity-dependent ligands activates a downstream factor more strongly when the ligands are in proximity than when they are distant from each other.

As used herein, “receptor component” refers to a polypeptide that functions as a receptor, by itself or as part of a complex. Thus a receptor component encompasses a polypeptide receptor and a polypeptide that functions as part of a receptor complex.

The term “synergy” or “synergistic” means a more than additive effect of a combination of two or more agents (e.g., polypeptides that are part of an enucleated erythroid cell) compared to their individual effects. In certain embodiments, synergistic activity is a more-than-additive effect of an enucleated erythroid cell comprising a first polypeptide and a second polypeptide, compared to the effect of an enucleated erythroid cell comprising the first polypeptide and an enucleated erythroid cell comprising the second polypeptide. In some embodiments, synergistic activity is present when a first agent produces a detectable level of an output X, a second agent produces a detectable level of the output X, and the first and second agents together produce a more-than-additive level of the output X.

As used herein, the term “variant” of a polypeptide refers to a polypeptide having at least one sequence difference compared to that polypeptide, e.g., one or more substitutions, insertions, or deletions. In some embodiments, the variant has at least 70%, 80%, 85%, 90%, 95%, 96%, 97%, 98%, or 99% identity to that polypeptide. A variant includes a fragment. In some embodiments, a fragment lacks up to 1, 2, 3, 4, 5, 10, 20, or 100 amino acids on the N-terminus, C-terminus, or both (each independently), compared to the full-length polypeptide.

### Exemplary Exogenous Polypeptides and Uses Thereof

In embodiments, the erythroid cell therapeutics described herein comprise one or more (e.g., 2, 3, 4, 5, 6, 10 or more) different exogenous agents, e.g., exogenous polypeptides, lipids, or small molecules. In some embodiments, an enucleated erythroid cell therapeutic comprises an exogenous fusion polypeptide comprising two or more different proteins described herein. In some embodiments, an enucleated erythroid cell comprises two or more different exogenous polypeptides described herein. In some embodiments, one or more (e.g., all) of the exogenous polypeptides are human polypeptides or fragments or variants thereof.

In some embodiments, the two or more polypeptides act on the same target, and in other embodiments, they act on two or more different targets. In some embodiments, the single target or plurality of targets is chosen from an endogenous human protein, a pathogen (e.g., a virus or bacterium), or a soluble factor (e.g., a polypeptide, small molecule, or cell-free nucleic acid).

One or more of the exogenous proteins may have post-translational modifications characteristic of eukaryotic cells, e.g., mammalian cells, e.g., human cells. In some embodiments, one or more (e.g., 2, 3, 4, 5, or more) of the exogenous proteins are glycosylated, phosphorylated, or both. In vitro detection of glycoproteins can be accomplished on SDS-PAGE gels and Western Blots using a modification of Periodic acid-Schiff (PAS) methods. Cellular localization of glycoproteins can be accomplished utilizing lectin fluorescent conjugates known in the art. Phosphorylation may be assessed by Western blot using phospho-specific antibodies.

Post-translation modifications also include conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition such as ADP-ribosylation, oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine). In embodiments, glycosylation includes the addition of a glycosyl group to arginine, asparagine, cysteine, hydroxylysine, serine, threonine, tyrosine, or tryptophan, resulting in a glycoprotein. In embodiments, the glycosylation comprises, e.g., O-linked glycosylation or N-linked glycosylation.

In some embodiments, one or more of the exogenous polypeptides is a fusion protein, e.g., is a fusion with an endogenous red blood cell protein or fragment thereof, e.g., a transmembrane protein, e.g., GPA or a transmembrane fragment thereof. In some embodiments, one or more of the exogenous polypeptides is fused with a domain that promotes dimerization or multimerization, e.g., with a second fusion exogenous polypeptide, which optionally comprises a dimerization domain. In some embodiments, the dimerization domain comprises a portion of an antibody molecule, e.g., an Fc domain or CH3 domain. In some embodiments, the first and second dimerization domains comprise knob-in-hole mutations (e.g., a T366Y knob and a Y407T hole) to promote heterodimerization. An exemplary human polypeptide, e.g., a human polypeptide selected from any of Tables 1-4, includes:

a) a naturally occurring form of the human polypeptide, e.g., a naturally occurring form of the human polypeptide that is not associated with a disease state;

b) the human polypeptide having a sequence appearing in a database, e.g., GenBank database, on Jul. 19, 2017, for example a naturally occurring form of the human polypeptide that is not associated with a disease state having a sequence appearing in a database, e.g., GenBank database, on Jul. 19, 2017;

c) a human polypeptide having a sequence that differs by no more than 1, 2, 3, 4, 5 or 10 amino acid residues from a sequence of a) or b);

d) a human polypeptide having a sequence that differs at no more than 1, 2, 3, 4, 5 or 10% its amino acids residues from a sequence of a) or b);

e) a human polypeptide having a sequence that does not differ substantially from a sequence of a) or b); or

f) a human polypeptide having a sequence of c), d), or e) that does not differ substantially in a biological activity, e.g., an enzymatic activity (e.g., specificity or turnover) or binding activity (e.g., binding specificity or affinity) from a human polypeptide having the sequence of a) or b). Candidate peptides under f) can be made and screened for similar activity as described herein and would be equivalent hereunder if expressed in enucleated erythroid cells as described herein).

In embodiments, an exogenous polypeptide comprises a human polypeptide or fragment thereof, e.g., all or a fragment of a human polypeptide of a), b), c), d), e), or f) of the preceding paragraph. In an embodiment, the exogenous polypeptide comprises a fusion polypeptide comprising all or a fragment of a human polypeptide of a), b), c), d), e), or f) of the preceding paragraph and additional amino acid sequence. In an embodiment the additional amino acid sequence comprises all or a fragment of human polypeptide of a), b), c), d), e), or f) of the preceding paragraph for a different human polypeptide.

The invention contemplates that functional fragments or variants thereof (e.g., a ligand-binding fragment or variant thereof or enzymatically active fragment or variant thereof, e.g., of the proteins listed in Table 1, 2, 3 or 4) can be made and screened for similar activity as described herein and would be equivalent hereunder if expressed in enucleated erythroid cells as described herein).

In embodiments, the two or more exogenous agents (e.g., polypeptides) have related functions that are agent-additive, agent-synergistic, multiplicative, independent function, localization-based, proximity-dependent, scaffold-based, multimer-based, pathway-based, or compensatory, as described herein. In some embodiments, more than one of these descriptors applies to a given erythroid cell.

Particular exogenous polypeptides that can be present or expressed in an erythroid cell are now described in greater detail.

### Exogenous CD14 Polypeptides

In some embodiments, the exogenous polypeptide comprises CD14 or a fragment or variant thereof. For example, an exogenous CD14 polypeptide can comprise a sequence of SEQ ID NO: 1, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the CD14 polypeptide has at least one activity characteristic of a CD14 polypeptide of SEQ ID NO: 1, e.g., it can bind a lipopolysaccharide (e.g., in the presence of a lipopolysaccharide-binding protein), e.g., with a Kd no greater than 10%, 20%, 50%, 2-fold, or 5-fold the Kd of a CD14 polypeptide of SEQ ID NO: 1 for the lipopolysaccharide. Functional CD14 polypeptides are described, Viriyakosol et al., “Structure-Function Analysis of CD14 as a Soluble Receptor for Lipopolysaccharide” The Journal of Biological Chemistry, 275, 3144-3149 (2000), which is herein incorporated by reference in its entirety. In some embodiments, the CD14 polypeptide comprises a CD14 extracellular domain (or fragment or variant thereof) and a membrane-anchor or transmembrane domain, e.g., a heterologous transmembrane domain, e.g., GPA.

In some embodiments, an erythroid cell described herein is contacted with, or comprises, a nucleic acid sequence (e.g., DNA or RNA) comprising a CD14 polypeptide described herein. In some embodiments, an erythroid cell described herein comprises a CD14 polypeptide as described herein and a TLR polypeptide (e.g., TLR2 or TLR4), e.g., as described herein.

### Exogenous TLR4 Polypeptides

In some embodiments, the exogenous polypeptide comprises TLR4 or a fragment or variant thereof. For example, an exogenous TLR4 polypeptide can comprise a sequence of SEQ ID NO: 2, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the TLR4 polypeptide has at least one activity characteristic of a TLR4 polypeptide of SEQ ID NO: 2, e.g., it can bind a lipopolysaccharide (e.g., in the presence of a lipopolysaccharide-binding protein), e.g., with a Kd no greater than 10%, 20%, 50%, 2-fold, or 5-fold the Kd of a TLR4 polypeptide of SEQ ID NO: 2 for the lipopolysaccharide. Functional TLR4 polypeptides are described, Ohto et al., “Structural Analyses of Human Toll-like Receptor 4 Polymorphisms D299G and T399I” The Journal of Biological Chemistry Vol. 287, No. 48, pp. 40611-40617, Nov. 23, 2012, and Anwar et al., “Structure-Activity Relationship in TLR4 Mutations: Atomistic Molecular Dynamics Simulations and Residue Interaction Network Analysis” Scientific Reports 7, Article number: 43807 (2017), each of which is incorporated herein by reference in its entirety. TLR structure is described in more detail in Jin et al., “Structures of the Toll-like Receptor Family and Its Ligand Complexes” Immunity, Volume 29, Issue 2, 15 August 2008, Pages 182-191, which is incorporated herein by reference in its entirety. In some embodiments, the TLR4 polypeptide comprises a TLR4 extracellular domain (or fragment or variant thereof) and a membrane-anchor or transmembrane domain, e.g., a heterologous transmembrane domain, e.g., GPA.

In some embodiments, an erythroid cell described herein is contacted with, or comprises, a nucleic acid sequence (e.g., DNA or RNA) comprising a TLR4 polypeptide described herein.

In some embodiments, an erythroid cell described herein comprises a TLR4 polypeptide as described herein and a CD14 polypeptide or a TLR2 polypeptide, e.g., as described herein.

### Exogenous TLR2 Polypeptides

In some embodiments, the exogenous polypeptide comprises TLR2 or a fragment or variant thereof. For example, an exogenous TLR2 polypeptide can comprise a sequence of SEQ ID NO: 5, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the TLR2 polypeptide has at least one activity characteristic of a TLR2 polypeptide of SEQ ID NO: 5, e.g., it can bind a lipopolysaccharide (e.g., in the presence of a lipopolysaccharide-binding protein), e.g., with a Kd no greater than 10%, 20%, 50%, 2-fold, or 5-fold the Kd of a TLR2 polypeptide of SEQ ID NO: 5 for the lipopolysaccharide. Functional TLR2 polypeptides are described, Kajava et al., “A Network of Hydrogen Bonds on the Surface of TLR2 Controls Ligand Positioning and Cell Signaling” J. Biol. Chem. 2010 Feb 26; 285(9): 6227-6234, which is herein incorporated by reference in its entirety. TLR structure is described in more detail in Jin et al., “Structures of the Toll-like Receptor Family and Its Ligand Complexes” Immunity, Volume 29, Issue 2, 15 August 2008, Pages 182-191, which is incorporated herein by reference in its entirety. In some embodiments, the TLR2 polypeptide comprises a TLR2 extracellular domain (or fragment or variant thereof) and a membrane-anchor or transmembrane domain, e.g., a heterologous transmembrane domain, e.g., GPA.

In some embodiments, an erythroid cell described herein is contacted with, or comprises, a nucleic acid sequence (e.g., DNA or RNA) comprising a TLR2 polypeptide described herein.

In some embodiments, an erythroid cell described herein comprises a TLR2 polypeptide as described herein and a CD14 polypeptide or a TLR4, e.g., as described herein.

### Exogenous CD4 Polypeptides

In some embodiments, the exogenous polypeptide comprises CD4 or a fragment or variant thereof. For example, an exogenous CD4 polypeptide can comprise a sequence of SEQ ID NO: 6, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the CD4 polypeptide has at least one activity characteristic of a CD4 polypeptide of SEQ ID NO: 6, e.g., it can bind HIV gp120, e.g., with a Kd no greater than 10%, 20%, 50%, 2-fold, or 5-fold the Kd of a CD4 polypeptide of SEQ ID NO: 6 for the gp120. Functional CD4 polypeptides are described, Moir et al., “CD4 deletion mutants evaluated for human immunodeficiency virus type 1 infectivity in a highly efficient system of expression and detection based on LTR-dependent reporter gene activation.” J. Virol. Methods. 1997 May; 65(2):209-17. In some embodiments, the CD4 polypeptide comprises a CD4 extracellular domain (or fragment or variant thereof) and a membrane-anchor or transmembrane domain, e.g., a heterologous transmembrane domain, e.g., GPA.

In some embodiments, an erythroid cell described herein is contacted with, or comprises, a nucleic acid sequence (e.g., DNA or RNA) comprising a CD4 polypeptide described herein.

In some embodiments, an erythroid cell described herein comprises a CD4 polypeptide as described herein and a CCR5 polypeptide, e.g., as described herein.

### Exogenous CCR5 Polypeptides

In some embodiments, the exogenous polypeptide comprises CCR5 or a fragment or variant thereof. For example, an exogenous CCR5 polypeptide can comprise a sequence of SEQ ID NO: 7 or 8, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the CCR5 polypeptide has at least one activity characteristic of a CCR5 polypeptide of SEQ ID NO: 7 or 8, e.g., it can bind HIV gp120, e.g., with a Kd no greater than 10%, 20%, 50%, 2-fold, or 5-fold the Kd of a CCRS polypeptide of SEQ ID NO: 7 or 8 for the gp120. Functional CCR5 polypeptides are described, in Kunstman et al., “Structure and Function of CC-Chemokine Receptor 5 Homologues Derived from Representative Primate Species and Subspecies of the Taxonomic Suborders Prosimii and Anthropoidea” J. Virol. 2003 Nov; 77(22): 12310-12318, which is herein incorporated by reference in its entirety

In some embodiments, an erythroid cell described herein is contacted with, or comprises, a nucleic acid sequence (e.g., DNA or RNA) comprising a CCR5 polypeptide described herein.

In some embodiments, an erythroid cell described herein comprises a CCR5 polypeptide as described herein and a CD4 polypeptide, e.g., as described herein.

### Asparaginase Polypeptides

In some embodiments, the exogenous polypeptide comprises asparaginase or a fragment or variant thereof. For example, an exogenous asparaginase polypeptide can comprise a sequence of SEQ ID NO: 8, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the asparaginase polypeptide has at least one activity characteristic of an asparaginase polypeptide of SEQ ID NO: 8, e.g., it can metabolize asparagine, e.g., with reaction rate constant at least 90%, 80%, 70%, 60%, or 50% of that of an asparaginase polypeptide of SEQ ID NO: 8. Asparagine metabolism can be measured, e.g., using an assay of Gervais and Foote, “Recombinant deamidated mutants of Erwinia chrysanthemi L-asparaginase have similar or increased activity compared to wild-type enzyme.” Mol. Biotechnol. 2014; 45(10): 865-877, which is herein incorporated by reference in its entirety. Functional asparaginase polypeptides are described, e.g., in Gervais and Foote, (supra), Nguyen et al. “Design and Characterization of Erwinia Chrysanthemi L-Asparaginase Variants with Diminished L-Glutaminase Activity.” J. Biol. Chem. 2016; 291(34): 17664-17676, and Moola et al. “Erwinia chrysanthemi L-asparaginase: epitope mapping and production of antigenically modified enzymes.” Biochemical Journal. 1994; 302(3): 921-927., each of which is herein incorporated by reference in its entirety. In embodiments, the asparaginase polypeptide comprises Erwinia chrysanthemi asparaginase or a fragment or variant thereof.

In some embodiments, an erythroid cell described herein is contacted with, or comprises, a nucleic acid sequence (e.g., DNA or RNA) comprising an asparaginase polypeptide described herein.

In some embodiments, an erythroid cell described herein comprises an asparaginase polypeptide as described herein and an asparagine transporter polypeptide (e.g., a SN2 or SAT2 polypeptide), e.g., as described herein.

### Sodium-Coupled Neutral Amino Acid Transporter 5 (SN2) Polypeptides

In some embodiments, the exogenous polypeptide comprises SN2 or a fragment or variant thereof. For example, an exogenous SN2 polypeptide can comprise a sequence of SEQ ID NO: 9, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the SN2 polypeptide has at least one activity characteristic of an asparaginase polypeptide of SEQ ID NO: 9, e.g., it can transport asparagine, e.g., at a rate at least 90%, 80%, 70%, 60%, or 50% of that of an SN2 polypeptide of SEQ ID NO: 9. Transport can be measured, e.g., using an assay of Nakanishi et al. “Structure, function, and tissue expression pattern of human SN2, a subtype of the amino acid transport system N.” Biochem. Biophys. Res. Commun. 2001 March; 281(5):1343-8, which is herein incorporated by reference in its entirety.

In some embodiments, an erythroid cell described herein is contacted with, or comprises, a nucleic acid sequence (e.g., DNA or RNA) comprising a SN2 polypeptide described herein.

In some embodiments, an erythroid cell described herein comprises a SN2 polypeptide as described herein and an asparaginase polypeptide, e.g., as described herein.

### Sodium-Coupled Neutral Amino Acid Transporter 2 (SAT2) Polypeptides

In some embodiments, the exogenous polypeptide comprises SAT2 or a fragment or variant thereof. For example, an exogenous SAT2 polypeptide can comprise a sequence of SEQ ID NO: 10, or a sequence with at least 80%, 85%, 90%, 95%, 96%, 97%, 98%, or 99% identity thereto, or a sequence with no more than 5, 4, 3, 2, or 1 amino acid alterations relative thereto, e.g., substitutions, insertions, or deletions. In embodiments, the SAT2 polypeptide has at least one activity characteristic of a SAT2 polypeptide of SEQ ID NO: 10, e.g., it can transport alpha-(methylamino)isobutyric acid, e.g., at a rate at least 90%, 80%, 70%, 60%, or 50% of that of a SAT2 polypeptide of SEQ ID NO: 10. Transport can be measured, e.g., using an assay of Hatanaka et al. “ Primary structure, functional characteristics and tissue expression pattern of human ATA2, a subtype of amino acid transport system A.” Biochim. Biophys. Acta. 2000 Jul. 31; 1467(1):1-6, which is herein incorporated by reference in its entirety.

In some embodiments, an erythroid cell described herein is contacted with, or comprises, a nucleic acid sequence (e.g., DNA or RNA) comprising a SAT2 polypeptide described herein.

In some embodiments, an erythroid cell described herein comprises a SAT2 polypeptide as described herein and an asparaginase polypeptide, e.g., as described herein.

Agent-Additive Configurations

When two or more agents (e.g., polypeptides) are agent-additive, the effect of the agents acting together is greater than the effect of either agent acting alone. In an embodiment, two agents have different (e.g., complementary) functions in the erythroid cell (e.g., on the erythroid cell surface) and act together to have a stronger effect (compared to either of the agents acting alone), e.g., a higher binding affinity for the target, or a greater degree of modulation of signal transduction by the target, e.g., a single target. In some embodiments, two or more agents each bind to the same target, e.g., to different epitopes within the same target protein.

In an embodiment the agents associate with one another, e.g., are members of a heterodimeric complex. In an embodiment, the agents have greater avidity for a target when acting together than when acting alone.

In some embodiments the agents bind to a pathogenic target with higher affinity than any one agent alone. For example, the agents may bind a virus or a bacterium or a fungus or a parasite and serve to clear the pathogenic target from circulation. In one embodiment, the agents comprise the proteins CD4 and CCR5, or functional variants or fragments thereof, for the capture of circulating HIV. As an example, the first polypeptide comprises CD4 (or an HIV-binding fragment or variant thereof) and the second polypeptide comprises CCR5 (or an HIV-binding fragment or variant thereof) to bind and/or inhibit circulating HIV. In embodiment, HIV enters a T cell using CD4 and CCR5 receptors. While not wishing to be bound by theory, in embodiments, an enucleated erythroid cell expressing CD4 and CCR5 serves as a decoy to prevent HIV from entering a T cell. In some embodiments, a treatment comprising a decoy is less prone to having the virus develop resistance, compared to conventional treatments, because a mutant virus that loses affinity for the decoy will also lack affinity for the corresponding human proteins and will be unable to dock at a human cell. In another embodiment the agents are chosen from CD14, MBL, rBPI21, and LPS binding protein, or any combination thereof, for the capture of circulating bacteria, fungi, or lipopolysaccharide. CD14 is a weak binder (on the order of micromolar affinity) alone, but this affinity is enhanced by the presence of LPS binding protein. In some embodiments, an enucleated erythroid cell comprising agents with affinity for bacteria is used to treat bacteremia.

In some embodiments, the two or more agents enable tighter binding to a target than either agent alone. In some embodiments, a heterodimer of receptor components bind to a target with higher affinity than either receptor component alone. Many signaling molecules form heterodimers or heteromultimers on the cell surface to bind to their ligand. Receptors, for example, can be heterodimers or heteromultimers.

In embodiments, an enucleated erythroid cell (e.g., a reticulocyte) herein expresses one or more (e.g., 2 or 3) of the receptors for the target molecule simultaneously. A table of cytokines and their receptors is provided herein as Table 1. In some embodiments, the agents are one or more cytokines. In some embodiments, an enucleated erythroid cell comprises one or more (e.g., 2, 3, 4, 5, or more) cytokines from Table 1 or receptor-binding variants or fragments thereof.

In some embodiments the first polypeptide comprises a cytokine or fragment or variant thereof, e.g., a cytokine of Table 1 or a fragment or variant thereof. In embodiments, the second polypeptide comprises a second cytokine or fragment or variant thereof, e.g., a cytokine of Table 1 or a fragment or variant thereof. In embodiments, one or more (e.g., 2 or all) of the cytokines are fused to transmembrane domains (e.g., a GPA transmembrane domain or other transmembrane domain described herein), e.g., such that the cytokine is on the surface of the erythroid cell. In embodiments, the erythroid cell further comprises a targeting moiety, e.g., a targeting moiety described in the section herein entitled “Localization Configurations.”

In some embodiments the agents are antibody molecules that bind cytokines, e.g., one or more cytokines of Table 1. In some embodiments, an enucleated erythroid cell comprises one or more (e.g., 2, 3, 4, 5, or more) cytokine receptor subunits from Table 1 or cytokine-binding variants or fragments thereof. In some embodiments, an enucleated erythroid cell comprises two or three (e.g., all) cytokine receptor subunits from a single row of Table 1 or cytokine-binding variants or functional fragments thereof. The cytokine receptors can be present on the surface of the erythroid cell. The expressed receptors typically have the wild type human receptor sequence or a variant or fragment thereof that is able to bind and sequester its target ligand. In embodiments, two or more cytokine receptor subunits are linked to each other, e.g., as a fusion protein.

TABLE 1
Cytokines and Receptors

| Name | Cytokine Receptor(s)(Da) and Form |
|---|---|
| Interleukins | |
| IL-1-like | |
| IL-lα | CD121a, CDw121b |
| IL-1β | CD121a, CDw121b |
| IL-1RA | CD121a |
| IL-18 | IL-18Rα, β |
| Common g chain (CD132) | |
| IL-2 | CD25, 122, 132 |
| IL-4 | CD124, 213a13, 132 |
| IL-7 | CD127, 132 |
| IL-9 | IL-9R, CD132 |
| IL-13 | CD213a1, 213a2, |
| IL-15 | IL-15Ra, CD122, 132 |
| Common b chain (CD131) | |
| IL-3 | CD123, CDw131 |
| IL-5 | CDw125, 131 |
| Also related | |
| GM-CSF | CD116, CDw131 |
| IL-6-like | |
| IL-6 | CD126, 130 |
| IL-11 | IL-11Ra, CD130 |
| Also related | |
| G-CSF | CD114 |
| IL-12 | CD212 |
| LIF | LIFR, CD130 |
| OSM | OSMR, CD130 |
| IL-10-like | |
| IL-10 | CDw210 |
| IL-20 | IL-20Rα, β |
| Others | |
| IL-14 | IL-14R |
| IL-16 | CD4 |
| IL-17 | CDw217 |
| Interferons | |
| IFN-α | CD118 |
| IFN-β | CD118 |
| IFN-γ | CDw119 |
| TNF | |
| CD154 | CD40 |
| LT-β | LTβR |
| TNF-α | CD120a, b |
| TNF-β (LT-α) | CD120a, b |
| 4-1BBL | CD137 (4-1BB) |
| APRIL | BCMA, TACI |
| CD70 | CD27 |
| CD153 | CD30 |
| CD178 | CD95 (Fas) |
| GITRL | GITR |
| LIGHT | LTbR, HVEM |
| OX40L | OX40 |
| TALL-1 | BCMA, TACI |
| TRAIL | TRAILR1-4 |
| TWEAK | Apo3 |
| TRANCE | RANK, OPG |
| TGF-β | |
| TGF-β1 | TGF-βR1 |
| TGF-β2 | TGF-βR2 |
| TGF-β3 | TGF-βR3 |
| Miscellaneous hematopoietins | |
| Epo | EpoR |
| Tpo | TpoR |
| Flt-3L | Flt-3 |
| SCF | CD117 |
| M-CSF | CD115 |
| MSP | CDw136 |

An enucleated erythroid cell can comprise a first exogenous polypeptide that interacts with a target and a second exogenous polypeptide (e.g., a protease) that modifies the target.

In embodiments, an effective amount of the enucleated erythroid cells comprising a first exogenous polypeptide and a second exogenous polypeptide is less than (e.g., less by 10%, 20%, 30%, 40%, 50%, 60%, 70%, 80%, 90%, 95%, 98%, 99%, 99.5%, 99.9%, or 99.99%) an effective amount of otherwise similar enucleated erythroid cells that lack the first exogenous polypeptide or lack the second exogenous polypeptide. In embodiments, the preselected amount is an effective dose or an in vitro effective amount of enucleated erythroid cells. In embodiments, the preselected amount (e.g., in vitro effective amount) is an amount that is effective in an assay, e.g., to convert at least 10%, 20%, 30%, 40%, 50%, 60%, 70%, 80%, or 90% of substrate into produce in a preselected amount of time, e.g., 1, 2, 3, 4, 5, or 6 hours. In embodiments, the preselected amount (e.g., in vitro effective amount) is effective to cleave or enzymatically convert at least 50% of a target (e.g., viral factor or bacterial factor) in 5 hours. The assay may measure, e.g., reduction in levels of soluble, unmodified (e.g., non-cleaved) target in a solution.

In embodiments, the reference value for targets is the number of targets in the peripheral blood of the subject at the time of administration. In embodiments (e.g., embodiments involving an in vitro effective amount of cells) the reference value for targets is the number of targets in a reaction mixture for an assay.

First Exogenous Polypeptide

In embodiments, the first exogenous polypeptide can bind a target.

In embodiments, the first exogenous polypeptide comprises a binding domain (e.g., a domain that binds the target) and a membrane anchor domain (e.g., a transmembrane domain, e.g., type I or type II red blood cell transmembrane domain). In embodiments, the membrane anchor domain is C-terminal or N-terminal of the binding domain. In embodiments, the transmembrane domain comprises GPA or a transmembrane portion thereof, e.g., as set out in SEQ ID NO: 33 herein or a transmembrane portion thereof, or a polypeptide having at least 70%, 75%, 80%, 85%, 90%, 95%, 96%, 97%, 98%, 99% identity to any of the foregoing. In embodiments, the GPA polypeptide is C-terminal of the binding domain.

In embodiments, the first exogenous polypeptide comprises an address moiety or targeting moiety described in WO2007030708, e.g., in pages 34-45 therein, which application is herein incorporated by reference in its entirety.

Other examples of proteins that can be suitably adapted for use as the first exogenous polypeptide include ligand binding domains of receptors, such as where the target is the receptor ligand. Conversely, the first exogenous polypeptide can comprise a receptor ligand where the target is the receptor. A target ligand can be a polypeptide or a small molecule ligand.

In a further embodiment, a first exogenous polypeptide may comprise a domain derived from a polypeptide that has an immunoglobulin-like fold, such as the 10th type III domain of human fibronectin (“Fn3”). See U.S. Pat. Nos. 6,673,901; 6,462,189. Fn3 is small (about 95 residues), monomeric, soluble and stable. It does not have disulfide bonds which permit improved stability in reducing environments. The structure may be described as a beta-sandwich similar to that of Ab VH domain except that Fn3 has seven beta-strands instead of nine. There are three loops on each end of Fn3; and the positions of three of these loops correspond to those of CDR1, 2 and 3 of the VH domain. The 94 amino acid Fn3 sequence is:

(SEQ ID NO: 34)
VSDVPRDLEWAATPTSLLISWDAPAVTVRYYRITYGETGGNSPVQEFTVP
GSKSTATISGLKPGVDYTITGYAVTGRGDSPASSKPISINYRT

The amino acid positions of the CDR-like loops will be defined as residues 23-30 (BC Loop), 52-56 (DE Loop) and 77-87 (FG Loop). Accordingly, one or more of the CDR-like loops may be modified or randomized, to generate a library of Fn3 binding domains which may then be screened for binding to a desired address binding site. See also PCT Publication WO0232925. Fn3 is an example of a large subfamily of the immunoglobulin superfamily (IgSF). The Fn3 family includes cell adhesion molecules, cell surface hormone and cytokine receptors, chaperonin, and carbohydrate-binding domains, all of which may also be adapted for use as binding agents. Additionally, the structure of the DNA binding domains of the transcription factor NF-kB is also closely related to the Fn3 fold and may also be adapted for use as a binding agent. Similarly, serum albumin, such as human serum albumin contains an immunoglobulin-like fold that can be adapted for use as a targeting moiety.

In still other embodiments, the first exogenous polypeptide can comprise an engineered polypeptide sequence that was selected, e.g., synthetically evolved, based on its kinetics and selectivity for binding to the address site. In embodiments, the sequence of the first exogenous polypeptide is designed using a screen or selection method, e.g., by phage display or yeast two-hybrid screen.

In some embodiments, the first exogenous polypeptide comprises a peptide ligand for a soluble receptor (and optionally the target comprises a soluble receptor), a synthetic peptide that binds a target, a complement regulatory domain (and optionally the target comprises a complement factor), or a ligand for a cell surface receptor (and optionally the target comprises the cell surface receptor).

Second Exogenous Polypeptide (e.g., Protease)

In embodiments, the second exogenous polypeptide (which modifies the target) is a factor set out in Table 5. In some embodiments, the protease is a protease set out in Table 5. In embodiments, the protease is a bacterial protease, a human protease, or a plant protease, or a fragment or variant thereof.

In embodiments, the second exogenous polypeptide (which modifies the target) is a protease. Exemplary proteases include those classified as Aminopeptidases; Dipeptidases; Dipeptidyl-peptidases and tripeptidyl peptidases; Peptidyl-dipeptidases; Serine-type carboxypeptidases; Metallocarboxypeptidases; Cysteine-type carboxypeptidases; Omegapeptidases; Serine proteinases; Cysteine proteinases; Aspartic proteinases; Metalloproteinases; or Proteinases of unknown mechanism.

Aminopeptidases include cytosol aminopeptidase (leucyl aminopeptidase), membrane alanyl aminopeptidase, cystinyl aminopeptidase, tripeptide aminopeptidase, prolyl aminopeptidase, arginyl aminopeptidase, glutamyl aminopeptidase, x-pro aminopeptidase, bacterial leucyl aminopeptidase, thermophilic aminopeptidase, clostridial aminopeptidase, cytosol alanyl aminopeptidase, lysyl aminopeptidase, x-trp aminopeptidase, tryptophanyl aminopeptidase, methionyl aminopeptidase, d-stereospecific aminopeptidase, and aminopeptidase. Dipeptidases include x-his dipeptidase, x-arg dipeptidase, x-methyl-his dipeptidase, cys-gly dipeptidase, glu-glu dipeptidase, pro-x dipeptidase, x-pro dipeptidase, met-x dipeptidase, non-stereospecific dipeptidase, cytosol non-specific dipeptidase, membrane dipeptidase, and beta-ala-his dipeptidase. Dipeptidyl-peptidases and tripeptidyl peptidases include dipeptidyl-peptidase I, dipeptidyl-peptidase II, dipeptidyl peptidase III, dipeptidyl-peptidase IV, dipeptidyl-dipeptidase, tripeptidyl-peptidase I, and tripeptidyl-peptidase II. Peptidyl-dipeptidases include peptidyl-dipeptidase A and peptidyl-dipeptidase B. Serine-type carboxypeptidases include lysosomal pro-x carboxypeptidase, serine-type D-ala-D-ala carboxypeptidase, carboxypeptidase C, and carboxypeptidase D. Metallocarboxypeptidases include carboxypeptidase A, carboxypeptidase B, lysine(arginine) carboxypeptidase, gly-X carboxypeptidase, alanine carboxypeptidase, muramoylpentapeptide carboxypeptidase, carboxypeptidase H, glutamate carboxypeptidase, carboxypeptidase M, muramoyltetrapeptide carboxypeptidase, zinc D-ala-D-ala carboxypeptidase, carboxypeptidase A2, membrane pro-x carboxypeptidase, tubulinyl-tyr carboxypeptidase, and carboxypeptidase T. Omegapeptidases include acylaminoacyl-peptidase, peptidyl-glycinamidase, pyroglutamyl-peptidase I, beta-aspartyl-peptidase, pyroglutamyl-peptidase II, n-formylmethionyl-peptidase, pteroylpoly-[gamma]-glutamate carboxypeptidase, gamma-glu-X carboxypeptidase, and acylmuramoyl-ala peptidase. Serine proteinases include chymotrypsin, chymotrypsin C, metridin, trypsin, thrombin, coagulation factor Xa, plasmin, enteropeptidase, acrosin, alpha-lytic protease, glutamyl, endopeptidase, cathepsin G, coagulation factor VIIa, coagulation factor IXa, cucumisi, prolyl oligopeptidase, coagulation factor XIa, brachyurin, plasma kallikrein, tissue kallikrein, pancreatic elastase, leukocyte elastase, coagulation factor XIIa, chymase, complement component c1r55, complement component c1s55, classical-complement pathway c3/c5 convertase, complement factor I, complement factor D, alternative-complement pathway c3/c5 convertase, cerevisin, hypodermin C, lysyl endopeptidase, endopeptidase la, gamma-reni, venombin AB, leucyl endopeptidase, tryptase, scutelarin, kexin, subtilisin, oryzin, endopeptidase K, thermomycolin, thermitase, endopeptidase SO, T-plasminogen activator, protein C, pancreatic endopeptidase E, pancreatic elastase II, IGA-specific serine endopeptidase, U-plasminogen, activator, venombin A, furin, myeloblastin, semenogelase, granzyme A or cytotoxic T-lymphocyte proteinase 1, granzyme B or cytotoxic T-lymphocyte proteinase 2, streptogrisin A, treptogrisin B, glutamyl endopeptidase II, oligopeptidase B, limulus clotting factor C, limulus clotting factor, limulus clotting enzyme, omptin, repressor lexa, bacterial leader peptidase I, and togavirin, flavirin. Cysteine proteinases include cathepsin B, papain, ficin, chymopapain, asclepain, clostripain, streptopain, actinide, cathepsin 1, cathepsin H, calpain, cathepsin T, glycyl, endopeptidase, cancer procoagulant, cathepsin S, picornain 3C, picornain 2A, caricain, ananain, stem bromelain, fruit bromelain, legumain, histolysain, and interleukin 1-beta converting enzyme. Aspartic proteinases include pepsin A, pepsin B, gastricsin, chymosin, cathepsin D, neopenthesin, renin, retropepsin, pro-opiomelanocortin converting enzyme, aspergillopepsin I, aspergillopepsin II, penicillopepsin, rhizopuspepsin, endothiapepsin, mucoropepsin, candidapepsin, saccharopepsin, rhodotorulapepsin, physaropepsin, acrocylindropepsin, polyporopepsin, pycnoporopepsin, scytalidopepsin A, scytalidopepsin B, xanthomonapepsin, cathepsin E, barrierpepsin, bacterial leader peptidase I, pseudomonapepsin, and plasmepsin. Metalloproteinases include atrolysin A, microbial collagenase, leucolysin, interstitial collagenase, neprilysin, envelysin, IgA-specific metalloendopeptidase, procollagen N-endopeptidase, thimet oligopeptidase, neurolysin, stromelysin 1, meprin A, procollagen C-endopeptidase, peptidyl-lys metalloendopeptidase, astacin, stromelysin 2, matrilysin gelatinase, aeromonolysin, pseudolysin, thermolysin, bacillolysin, aureolysin, coccolysin, mycolysin, beta-lytic metalloendopeptidase, peptidyl-asp metalloendopeptidase, neutrophil collagenase, gelatinase B, leishmanolysin, saccharolysin, autolysin, deuterolysin, serralysin, atrolysin B, atrolysin C, atroxase, atrolysin E, atrolysin F, adamalysin, horrilysin, ruberlysin, bothropasin, bothrolysin, ophiolysin, trimerelysin I, trimerelysin II, mucrolysin, pitrilysin, insulysin, O-syaloglycoprotein endopeptidase, russellysin, mitochondrial, intermediate, peptidase, dactylysin, nardilysin, magnolysin, meprin B, mitochondrial processing peptidase, macrophage elastase, choriolysin, and toxilysin. Proteinases of unknown mechanism include thermopsin and multicatalytic endopeptidase complex. In embodiments, the second exogenous polypeptide comprises a fragment or variant of any of the foregoing.

In embodiments, the second exogenous polypeptide comprises an IdeS polypeptide. In some embodiments, the IdeS polypeptide comprises the sequence set out below as SEQ ID NO: 35 or a proteolytically active fragment of the sequence of SEQ ID NO: 35 (e.g., a fragment of at least 100, 150, 200, 250, or 300 amino acids) or a sequence having at least 70%, 75%, 80%, 85%, 90%, 95%, 96%, 97%, 98%, 99% identity to any of the foregoing. In some embodiments involving nucleic acids, the nucleic acid encodes an IdeS polypeptide having the sequence set out below as SEQ ID NO: 35, or a proteolytically active fragment thereof, or a sequence having at least 70%, 75%, 80%, 85%, 90%, 95%, 96%, 97%, 98%, 99% identity to any of the foregoing.

IdeS Polypeptide:

(SEQ ID NO: 35)
DSFSANQEIRYSEVTPYHVTSVWTKGVTPPAKFTQGEDVFHAPYVANQGW
YDITKTFNGKDDLLCGAATAGNMLHWWFDQNKEKIEAYLKKHPDKQKIMF
GDQELLDVRKVINTKGDQTNSELFNYFRDKAFPGLSARRIGVMPDLVLDM
FINGYYLNVYKTQTTDVNRTYQEKDRRGGIFDAVFTRGDQSKLLTSRHDF
KEKNLKEISDLIKKELTEGKALGLSHTYANVRINHVINLWGADFDSNGNL
KAIYVTDSDSNASIGMKKYFVGVNSAGKVAISAKEIKEDNIGAQVLGLFT
LSTGQDSWNQTN

In embodiments, the second exogenous polypeptide comprises a modifier domain (e.g., a protease domain, e.g., an IdeS polypeptide) and a membrane anchor domain (e.g., a transmembrane domain, e.g., type I or type II red blood cell transmembrane domain). In embodiments, the membrane anchor domain is C-terminal or N-terminal of the modifier (e.g., protease) domain. In embodiments, the transmembrane domain comprises GPA or a transmembrane portion thereof. In embodiments, the GPA polypeptide has a sequence of:

(SEQ ID NO: 33)
LSTTEVAMHTSTSSSVTKSYISSQTNDTHKRDTYAATPRAHEVSEISVRT
VYPPEEETGERVQLAHHFSEPEITLIIFGVMAGVIGTILLISYGIRRLIK
KSPSDVKPLPSPDTDVPLSSVEIENPETSDQ

or a transmembrane portion thereof, or a polypeptide having at least 70%, 75%, 80%, 85%, 90%, 95%, 96%, 97%, 98%, 99% identity to any of the foregoing. In embodiments, the GPA polypeptide is C-terminal of the modifier (e.g., protease) domain.

In some embodiments, a linker is disposed between the IdeS polypeptide and the transmembrane polypeptide, e.g., a glycine-serine linker, e.g., a linker comprising a sequence of GGSGGSGG (SEQ ID NO: 36) and/or GGGSGGGS (SEQ ID NO: 37). In some embodiments, the exogenous polypeptide, e.g., the second exogenous polypeptide, e.g., a protease, e.g., IdeS polypeptide, comprises a leader sequence, e.g., a GPA leader sequence, e.g., MYGKIIFVLLLSEIVSISA (SEQ ID NO: 38).

In some embodiments, the exogenous polypeptide, e.g., the second exogenous polypeptide further comprises a tag, e.g., an HA tag or a FLAG tag.

In some embodiments, the protease (e.g., immunoglobulin degrading enzyme, e.g., immunoglobulin-G degrading enzyme, e.g., IdeS) cleaves an immunoglobulin at a hinge region, a CH2 region, or between a hinge and CH2 region. In embodiments, the protease cleaves an immunoglobulin at one of the sequences below, e.g., between the two italicized glycines or the italicized alanine and glycine in the sequences below.

| (SEQ ID NO: 39) | |
|---|---|
| Human IgG1 Hinge/CH2 Sequence | CPPCPAPELLGGPSVF |
| (SEQ ID NO: 40) | |
| Human IgG2 Hinge/CH2 Sequence | CPPCPAPPVAGPSVF |
| (SEQ ID NO: 41) | |
| Human IgG3 Hinge/CH2 Sequence | CPRCPAPELLGGPSVF |
| (SEQ ID NO: 42) | |
| Human IgG4 Hinge/CH2 Sequence | AHHAQAPEFLGGPSVF |

In embodiments, the protease (e.g., a bacterial protease) cleaves IgG, e.g., IdeS or IgA protease.

In embodiments, the protease (e.g., a papain family protease, e.g., papain) cleaves an immunoglobulin between the Fc and Fab regions, e.g., a histidine-threonine bond between positions 224 and 225 of the heavy chain and/or a glutamic acid-leucine bond between positions 233 and 234 of the heavy chain.

In embodiments, the protease or other modifier acts on a target listed in Table 5 or Table 6.

In embodiments, the protease or other modifier acts on (e.g., inactivates or inhibits) a TNF molecule (such as TNF-alpha).

In embodiments, the second exogenous polypeptide comprises a catalytic moiety described in WO2007030708, e.g., in pages 45-46 therein, which application is herein incorporated by reference in its entirety.

The second exogenous polypeptide can comprise a moiety capable of acting on a target to induce a chemical change, thereby modulate its activity, e.g., a moiety capable of catalyzing a reaction within a target. The second exogenous polypeptide can comprise a naturally occurring enzyme, an active (e.g., catalytically active) fragment thereof, or an engineered enzyme, e.g., a protein engineered to have an enzymatic activity, such as a protein designed to contain a serine protease active motif. A catalytic domain of a second exogenous polypeptide may comprise the arrangement of amino acids that are effective to induce the desired chemical change in the target. They may be N-terminal or C-terminal truncated versions of natural enzymes, mutated versions, zymogens, or complete globular domains.

The second exogenous polypeptide can comprise an enzymatically active site that alone is promiscuous, binding with a cleavage site it recognizes on many different biomolecules, and may have relatively poor reaction kinetics. In embodiments, the first exogenous polypeptide supplies or improves specificity by increasing the local concentration of target near the second exogenous polypeptide.

The second exogenous polypeptide can, in embodiments, modify the target so that it is recognized and acted upon by another enzyme (e.g., an enzyme that is already present in a subject). In an embodiment, the second exogenous polypeptide comprises a moiety that alters the structure of the target so that its activity is inhibited or upregulated. Many naturally occurring enzymes activate other enzymes, and these can be exploited in accordance with the compositions and methods described herein.

The second exogenous polypeptide can comprise a protease, a glycosidase, a lipase, or other hydrolases, an amidase (e.g., N-acetylmuramoyl-L-alanine amidase, PGRP-L amidase), or other enzymatic activity, including isomerases, transferases (including kinases), lyases, oxidoreductases, oxidases, aldolases, ketolases, glycosidases, transferases and the like. In embodiments, the second exogenous polypeptide comprises human lysozyme, a functional portion of a human lysozyme, a human PGRP-L, a functional portion of a human PGRP-L, a phospholipase A2, a functional portion of a phospholipase A2, or a matrix metalloproteinase (MMP) extracellular enzyme such as MMP-2 (gelatinase A) or MMP-9 (gelatinase B).

In embodiments, the second exogenous polypeptide is a serine proteinase, e.g., of the chymotrypsin family which includes the mammalian enzymes such as chymotrypsin, trypsin or elastase or kallikrein, or the substilisin family which includes the bacterial enzymes such as subtilisin. The general three-dimensional structure is different in the two families but they have the same active site geometry and catalysis proceeds via the same mechanism. The serine proteinases exhibit different substrate specificities which are related to amino acid substitutions in the various enzyme subsites interacting with the substrate residues. Three residues which form the catalytic triad are important in the catalytic process: His-57, Asp-102 and Ser-195 (chymotrypsinogen numbering).

In embodiments, the second exogenous polypeptide is a cysteine proteinase which includes the plant proteases such as papain, actinidin or bromelain, several mammalian lysosomal cathepsins, the cytosolic calpains (calcium-activated), and several parasitic proteases (e.g., Trypanosoma, Schistosoma). Papain is the archetype and the best studied member of the family. Like the serine proteinases, catalysis proceeds through the formation of a covalent intermediate and involves a cysteine and a histidine residue. The essential Cys-25 and His-159 (papain numbering) play the same role as Ser-195 and His-57 respectively. The nucleophile is a thiolate ion rather than a hydroxyl group. The thiolate ion is stabilized through the formation of an ion pair with neighboring imidazolium group of His-159. The attacking nucleophile is the thiolate-imidazolium ion pair in both steps and then a water molecule is not required.

In embodiments, the second exogenous polypeptide is an aspartic proteinase, most of which belong to the pepsin family. The pepsin family includes digestive enzymes such as pepsin and chymosin as well as lysosomal cathepsins D, processing enzymes such as renin, and certain fungal proteases (penicillopepsin, rhizopuspepsin, endothiapepsin). A second family comprises viral proteinases such as the protease from the AIDS virus (HIV) also called retropepsin. In contrast to serine and cysteine proteinases, catalysis by aspartic proteinases does not involve a covalent intermediate, though a tetrahedral intermediate exists. The nucleophilic attack is achieved by two simultaneous proton transfers: one from a water molecule to the dyad of the two carboxyl groups and a second one from the dyad to the carbonyl oxygen of the substrate with the concurrent CO—NH bond cleavage. This general acid-base catalysis, which may be called a “push-pull” mechanism leads to the formation of a non-covalent neutral tetrahedral intermediate.

In embodiments, the second exogenous polypeptide is a metalloproteinase, which can be found in bacteria, fungi as well as in higher organisms. They differ widely in their sequences and their structures but the great majority of enzymes contain a zinc (Zn) atom which is catalytically active. In some cases, zinc may be replaced by another metal such as cobalt or nickel without loss of the activity. Bacterial thermolysin has been well characterized and its crystallographic structure indicates that zinc is bound by two histidines and one glutamic acid. Many enzymes contain the sequence HEXXH, which provides two histidine ligands for the zinc whereas the third ligand is either a glutamic acid (thermolysin, neprilysin, alanyl aminopeptidase) or a histidine (astacin). Other families exhibit a distinct mode of binding of the Zn atom. The catalytic mechanism leads to the formation of a non-covalent tetrahedral intermediate after the attack of a zinc-bound water molecule on the carbonyl group of the scissile bond. This intermediate is further decomposed by transfer of the glutamic acid proton to the leaving group.

In embodiments, the second exogenous polypeptide comprises an isomerase (e.g., an isomerase that breaks and forms chemical bonds or catalyzes a conformational change). In embodiments, the isomerase is a racemase (e.g., amino acid racemase), epimerase, cis-trans isomerase, intramolecular oxidoreductase, intramolecular transferase, or intramolecular lyase.

In embodiments, the second exogenous protease comprises a chaperone, or an active variant or fragment thereof. For instance, the chaperone can be a general chaperone (e.g., GRP78/BiP, GRP94, GRP170), a lectin chaperone (e.g., calnexin or calreticulin), a non-classical molecular chaperone (e.g., HSP47 or ERp29), a folding chaperone (e.g., PDI, PPI, or ERp57), a bacterial or archaeal chaperone (e.g., Hsp60, GroEL/GroES complex, Hsp70, DnaK, Hsp90, HtpG, Hsp100, Clp family (e.g., ClpA and ClpX), Hsp104). In embodiments, the enucleated erythrocyte comprises a co-chaperone, or an active variant or fragment thereof, e.g., immunophilin, Stil, p50 (Cdc37), or Ahal. In embodiments, the molecular chaperone is a chaperonin.

Candidates for the second exogenous protein (which modifies a target) can be screened based on their activity. Depending on the specific activity of each molecule being tested, an assay appropriate for that molecule can be used. For example, if the second exogenous protein is a protease, the assay used to screen the protease can be an assay to detect cleavage products generated by the protease, e.g., a chromatography or gel electrophoresis based assay.

In an example, the second exogenous polypeptide may have kinase activity. An assay for kinase activity could measure the amount of phosphate that is covalently incorporated into the target of interest. For example, the phosphate incorporated into the target of interest could be a radioisotope of phosphate that can be quantitated by measuring the emission of radiation using a scintillation counter.

Targets (e.g., Complement Pathway Factors, Clotting Factors, and Amino Acids) and Indications

In embodiments, the target is a target listed in Table 5 or Table 6.

In embodiments, the target is an immune checkpoint molecule selected from PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 or TGF beta. In embodiments, the target is an inhibitory ligand listed in Table 3, and the first exogenous polypeptide optionally comprises a binding domain from a corresponding target receptor of Table 3. In some embodiments, the target is a target receptor of Table 3, and the first exogenous polypeptide optionally comprises a binding domain from a corresponding inhibitory ligand of Table 3. In some embodiments, the second exogenous polypeptide comprises a protease that cleaves an immune checkpoint molecule, e.g., trypsin. In embodiments, e.g., for treating an infectious disease, a T cell is activated or prevented from being inactivated, e.g., by contacting its receptor (e.g., a receptor of Table 3) with a molecule that blocks T cell inhibition.

In embodiments, the target is an antibody e.g., a human antibody.

Engineered erythroid cells described herein can also be used to treat a subject that has antibodies against a drug. The erythroid cell can reduce levels of anti-drug antibodies in a subject, and can optionally further comprise a therapeutic protein that treats the disease. For instance, the erythroid cell comprises a first exogenous polypeptide that binds a target, e.g., wherein the target is an anti-drug antibody. The erythroid cell can further comprise a second exogenous polypeptide (e.g., a protease) that inactivates, e.g., cleaves the target. The erythroid cell may optionally further comprise a third exogenous polypeptide, e.g., a therapeutic protein that treats the same disease as the prior therapeutic to which the subject developed anti-drug antibodies, e.g., a therapeutic protein which is the same as or different from the prior therapeutic to which the subject developed anti-drug antibodies. In embodiments, the subject comprises anti-drug antibodies against erythropoietin, an anti-TNF antibody molecule (adalimumab or infliximab), an anti-EGFR antibody (e.g., cetuximab), an anti-CD20 antibody molecule, insulin, an anti-alpha4 integrin antibody molecule (e.g., natalizumab), or an interferon, e.g., IFNβ1a or IFNβ1b. In embodiments, the first polypeptide comprises a molecule that binds inflamed tissue (e.g., an anti-MAdCAM-1 antibody molecule) and the second polypeptide comprises an antiviral or anti-bacterial protein, e.g., wherein the target tissue is inflamed tissue, e.g., infected tissue. In some embodiments, the patient may be tested for the presence of anti-drug antibodies, e.g., for the presence of neutralizing anti-drug antibodies, before, during and/or after administration of the engineered erythroid cells described herein.

Agent-Synergistic Configurations

When two or more agents (e.g., polypeptides) are agent-synergistic, the agents act on two or more different targets within a single pathway. In an embodiment, the action of the two or more agents together is greater than the action of any of the individual agents. For example, the first and second polypeptides are ligands for cellular receptors that signal to the same downstream target. For example, the first exogenous polypeptide comprises a ligand for a first target cellular receptor, and the second exogenous polypeptide comprises a ligand for a second target cellular receptor, e.g., which first and second target cellular receptors signal to the same downstream target. In embodiments, the first exogenous polypeptide acts on the first target and the second exogenous polypeptide acts on the second target simultaneously, e.g., there is some temporal overlap in binding of the first exogenous polypeptide to the first target and binding of the second exogenous polypeptide to the second target. In some embodiments the simultaneous action generates a synergistic response of greater magnitude than would be expected when either target is acted on alone or in isolation.

In an embodiment, the first and second polypeptides are ligands for a first cellular receptor and a second cellular receptor that mediates apoptosis. In an embodiment the agents comprise two or more TRAIL receptor ligands, e.g., wild-type or mutant TRAIL polypeptides, or antibody molecules that bind TRAIL receptors, and induce apoptosis in a target cell, e.g., an infected cell. In some embodiments, a erythroid cell comprising TRAIL receptor ligands further comprises a targeting moiety, e.g., a targeting moiety described herein. In an embodiment the first target and the second target interacts with the same substrate, e.g., a substrate protein. In an embodiment the first target and the second target interact with different substrates.

TRAIL (TNF-related apoptosis inducing ligand) is a member of the TNF family that induces apoptosis. TRAIL has at least two receptors, TRAIL R1 and TRAIL R2. TRAIL receptor agonists, e.g., mutants of TRAIL that bind one or more of the receptors, or antibody molecules that bind one or both of TRAIL R1 or TRAIL R2 (see, e.g. Gasparian et al., Apoptosis 2009 Jun. 14(6), Buchsbaum et al. Future Oncol. 2007 Aug. 3(4)), have been developed as a clinical therapy for a wide range of cancers. Clinical trials of TRAIL receptor agonists have failed for, among other reasons, the fact that many primary cancers are not sensitive to signaling through a single receptor but rather require engagement of both receptors to induce cytotoxicity (Marconi et al., Cell Death and Disease (2013) 4, e863). In one embodiment the agents expressed on the engineered erythroid cell are single receptor-specific TRAIL agonists that, in combination, enable the cell to engage and agonize both TRAIL receptors simultaneously, thus leading to a synergistic induction of apoptosis of a target cell. Thus, in some embodiments, the enucleated erythroid cell comprises on its surface a first polypeptide that binds TRAIL R1 and a second polypeptide that binds TRAIL R2. In embodiments, each polypeptide has a Kd for TRAIL R1 or TRAIL R2 that is 2, 3, 4, 5, 10, 20, 50, 100, 200, or 500-fold stronger than the Kd for the other receptor. While not wishing to be bound by theory, in some embodiments an enucleated erythroid cell comprising a TRAIL R1-specific ligand and a TRAIL R2-specific ligand promote better heterodimerization of TRAIL R1 and TRAIL R2 than an enucleated erythroid cell comprising a ligand that binds to TRAIL R1 and TRAIL R2 with about the same affinity.

In some embodiments, one, two, or more of the exogenous polypeptides are members of the TNF superfamily. In some embodiments, the exogenous polypeptides bind to one or both of death receptors DR4 (TRAIL-R1) and DRS (TRAIL-R2). In some embodiments, the exogenous polypeptides bind to one or more of TNFRSF10A/TRAILR1, TNFRSF10B/TRAILR2, TNFRSF10C/TRAILR3, TNFRSF10D/TRAILR4, or TNFRSF11B/OPG. In some embodiments, the exogenous polypeptides activate one or more of MAPK8/JNK, caspase 8, and caspase 3.

In some embodiments, a TRAIL polypeptide is a TRAIL agonist having a sequence of any of SEQ ID NOS: 43-47 herein, or a sequence with at least 70%, 80%, 85%, 90%, 95%, 96%, 97%, 98%, or 99% identity thereto. Sequence identity is measured, e.g., by BLAST (Basic Local Alignment Search Tool). SEQ ID Nos. 43-47 are further described in Mohr et al. BMC Cancer (2015) 15:494), which is herein incorporated by reference in its entirety.

Soluble TRAIL variant DR4-1
SEQ ID NO: 43
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRRRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG
Soluble TRAIL variant DR4-2
SEQ ID NO: 44
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRRGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG
Soluble TRAIL variant DR4-3
SEQ ID NO: 45
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRRRSNTLSSPNSKNEKALGIK
INSWESSRRGHSFLSNLHLRNGELVIHEKGFYYIYSQTYFRFQEEIKENT
KNDKQMVQYIYKYTDYPDPILLMKSARNSCWSKDAEYGLYSIYQGGIFEL
KENDRIFVSVTNEHLIDMDHEASFFGAFLVG
Soluble TRAIL variant DR5-1
SEQ ID NO: 46
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMHHEASFFGAFLVG
Soluble TRAIL variant DR5-2
SEQ ID NO: 47
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQERIKENT
KNDKQMVQYIYKYTSYPDPILLMKSARNSCWSKDAEYGLYSIYQGGIFEL
KENDRIFVSVTNEHLIDMHHEASFFGAFLVG

All combinations of the TRAIL receptor ligands are envisioned. In some embodiments, the first and second agents comprise SEQ ID NO: 43 and SEQ ID NO: 44; SEQ ID NO: 43 and SEQ ID NO: 45; SEQ ID NO: 434 and SEQ ID NO: 36; SEQ ID NO: 43 and SEQ ID NO: 47; SEQ ID NO: 44 and SEQ ID NO: 45; SEQ ID NO: 44 and SEQ ID NO: 46; SEQ ID NO: 44 and SEQ ID NO: 47; SEQ ID NO: 45 and SEQ ID NO: 46; SEQ ID NO: 45 and SEQ ID NO: 47; or SEQ ID NO: 45 and SEQ ID NO: 48, or a fragment or variant of any of the foregoing.

In some embodiments, the TRAIL receptor ligand comprises an antibody molecule. In embodiments, the antibody molecule recognizes one or both of TRAIL R1 and TRAIL R2. The antibody molecule may be, e.g., Mapatumumab (human anti-DR4 mAb), Tigatuzumab (humanized anti-DRS mAb), Lexatumumab (human anti-DRS mAb), Conatumumab (human anti-DRS mAb), or Apomab (human anti-DRS mAb), or a fragment or variant thereof, e.g., a variant having the same CDRs as any of the aforementioned antibodies, e.g., by the Chothia or Kabat definitions. In some embodiments, the enucleated erythroid cell comprises two or more (e.g., three, four, five, or more) different antibody molecules that bind a TRAIL receptor. In some embodiments, the enucleated erythroid cell comprises at least one antibody molecule that binds a TRAIL receptor and at least one TRAIL polypeptide.

In some embodiments, the agents are modulators of a multi-step pathway that act agent-synergistically by targeting upstream and downstream steps of the pathway, e.g., simultaneously.

In one embodiment, the target pathway is the complement cascade, which has several parallel activation paths (classical, alternative, lectin pathways) and multiple auto-catalytic enzymes to enhance its potency in responding to infection and leading to membrane-attack complex formation (see, e.g. Bu et al., Clin. Dev. Immunol. 2012; 2012: 370426). In embodiments, an erythroid cell described herein is administered to a subject having an infectious disease in order to activate the complement pathway. In embodiments, the first exogenous polypeptide comprises a first complement factor (e.g., an activated complement factor) and the second exogenous polypeptide comprises a second complement factor (e.g., an activated complement factor). The exogenous polypeptide can also comprise a fragment or variant of any of the complement inhibitors described herein. In embodiments, the complement factor or factors are chosen from C1, C2a, C4b, C3, C3a, C3b, C5, C5a, C5b, C6, C7, C8, or C9.

Multiplicative Configurations

When two or more agents (e.g., polypeptides) are multiplicative, a first agent acts on a first molecule that is part of a first pathway and a second agent acts on a second molecule that is part of a second pathway, which pathways act in concert toward a desired response.

In some embodiments, the desired response is cell death, e.g., of an infected cell. In some embodiments, the agents trigger multiple T cell activation pathways to induce an immune response. In some embodiments, the engineered erythroid cell promotes T cell proliferation. In embodiments, one or more (e.g., 2, 3, 4, or 5 or more) T cell activation ligands comprise a ligand of Table 2 or a T-cell activating variant (e.g., fragment) thereof. In embodiments, one or more (e.g., 2, 3, 4, or 5 or more) T cell activation ligands comprise an antibody molecule that binds a target receptor of Table 2 or a T-cell activating variant (e.g., fragment) thereof. In some embodiments, the first and second polypeptides comprise different T cell activation ligands, e.g. CD80, 41BB-ligand, CD86, or any combination thereof, to stimulate T cells. In some embodiments, the enucleated erythroid cell comprises 4-1BBL, OX4OL, and CD4OL, or fragments or variants thereof. In embodiments, these proteins signal through complementary activation pathways. In some embodiments the ligands are activating cytokines, interferons, or TNF family members (e.g., of Table 1), e.g. IFNa, IL2, or IL6 or any combination thereof. In some embodiments the agents are combinations of the above classes of molecules. The agents can be derived from endogenous ligands or antibody molecules to the target receptors.

TABLE 2
T cell activation

| Activating Ligand | Target Receptor on T cell |
|---|---|
| B7-H2 (e.g., Accession Number | ICOS, CD28 (e.g., Accession Number |
| NP_056074.1) | NP_006130.1) |
| B7-1 (e.g., Accession Number | CD28 (e.g., Accession Number |
| NP_005182.1) | NP_006130.1) |
| B7-2 (e.g., Accession Number | CD28 (e.g., Accession Number |
| AAA86473) | NP_006130.1) |
| CD70 (e.g., Accession Number | CD27 (e.g., Accession Number |
| NP_001243.1) | NP_001233.1) |
| LIGHT (e.g., Accession Number | HVEM (e.g., Accession Number |
| NP_003798.2) | AAQ89238.1) |
| HVEM (e.g., Accession Number | LIGHT (e.g., Accession Number |
| AAQ89238.1) | NP_003798.2) |
| CD40L (e.g., Accession Number | CD40 (e.g., Accession Number |
| BAA06599.1) | NP_001241.1) |
| 4-1BBL (e.g., Accession Number | 4-1BB (e.g., Accession |
| NP_003802.1) | NP_001552.2) |
| OX40L (e.g., Accession Number | OX40 (e.g., Accession Number |
| NP_003317.1) | NP_003318.1) |
| TL1A (e.g., Accession Number | DR3 (e.g., Accession Number |
| NP_005109.2) | NP_683866.1) |
| GITRL (e.g., Accession Number | GITR (e.g., Accession Number |
| NP_005083.2) | NP_004186.1) |
| CD30L (e.g., Accession Number | CD30 (e.g., Accession Number |
| NP_001235.1), | NP_001234.3) |
| TIM4 (e.g., Accession Number | TIM1 (e.g., Accession Number |
| NP_612388.2) | NP_036338.2) |
| SLAM (e.g., Accession Number | SLAM (e.g., Accession Number |
| AAK77968.1) | AAK77968.1) |
| CD48 (e.g., Accession Number | CD2 (e.g., Accession Number |
| CAG33293.1) | NP 001315538.1) |
| CD58 (e.g., Accession Number | CD2 (e.g., Accession Number |
| CAG33220.1) | NP_001315538.1) |
| CD155 (e.g., Accession Number | CD226 (e.g., Accession Number |
| NP_001129240.1) | NP_006557.2) |
| CD112 (e.g., Accession Number | CD226 (e.g., Accession Number |
| NP_001036189.1) | NP_006557.2) |
| CD137L (e.g., Accession Number | CD137 (e.g., Accession |
| NP_003802.1) | NP_001552.2) |

In some embodiments, an anti-IL6 or TNFa antibody molecule comprises a sequence of either of SEQ ID NO: 48 or 49 herein, or a sequence with at least 70%, 80%, 85%, 90%, 95%, 96%, 97%, 98%, or 99% identity thereto.

Anti-IL6 scFv
SEQ ID NO: 48
EVQLVESGGGLVQPGGSLRLSCAASGFNFNDYFMNWVRQAPGKGLEWVAQ
MRNKNYQYGTYYAESLEGRFTISRDDSKNSLYLQMNSLKTEDTAVYYCAR
ESYYGFTSYWGQGTLVTVSSGGGGSGGGGSGGGGSDIQMTQSPSSLSASV
GDRVTITCQASQDIGISLSWYQQKPGKAPKLLIYNANNLADGVPSRFSGS
GSGTDFTLTISSLQPEDFATYYCLQHNSAPYTFGQGTKLEIKR
Anti-TNFα scFv
SEQ ID NO: 49
EVQLVESGGGLVQPGRSLRLSCAASGFTFDDYAMHWVRQAPGKGLEWVSA
ITWNSGHIDYADSVEGRFTISRDNAKNSLYLQMNSLRAEDTAVYYCAKVS
YLSTASSLDYWGQGTLVTVSSGGGGSGGGGSGGGGSDIQMTQSPSSLSAS
VGDRVTITCRASQGIRNYLAWYQQKPGKAPKLLIYAASTLQSGVPSRFSG
SGSGTDFTLTISSLQPEDVATYYCQRYNRAPYTFGQGTKVEIK

As another example, the first and second polypeptides comprise a T cell activating ligand and an agent which inhibits an immune inhibitory molecule (e.g., an immune inhibitory receptor), e.g. CD80 and anti-PD1, in an immuno-oncology setting. In another embodiment, one agent is an activating 4-1BBL, or fragment or variant thereof, and a second agent an antibody molecule that blocks PD1 signaling (e.g., an antibody molecule to PD1 or PD-L1). Thus, in embodiments, a target T cell is both activated and prevented from being repressed. Examples of agents that inhibit an immune inhibitory molecule include inhibitors of (e.g., antibody molecules that bind) PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta, or a functional variant (e.g., fragment) thereof. In some embodiments, the agent that inhibits an immune inhibitory molecule is an inhibitor of an inhibitory ligand of Table 3, or an inhibitory fragment or variant thereof. In some embodiments, the agent that inhibits an immune inhibitory molecule is an antibody molecule that binds a target receptor of Table 3, or a fragment or variant thereof.

TABLE 3
T cell inhibition

| Inhibitory Ligand | Target Receptor on T cell |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN9 | TIM3 |
| CD48, TIM4 | TIM4R |
| CD48 | 2B4 |
| CD155, CD112, CD113 | TIGIT |
| PDL1 | PD1 |

In embodiments, an engineered erythroid cell targets multiple T cell inhibitory pathways in combination (e.g., as described in Table 3), e.g., using ligands or antibody molecules, or both, co-expressed on an engineered erythroid cell.

In some embodiments, one of the agents for treating an infectious disease comprises an activating cytokine, e.g., IL-2, IL-12, or another activating cytokine of Table 1, or a fragment or variant thereof.

In some embodiments the objective is to activate or to inhibit T cells. To ensure that T cells are preferentially targeted over other immune cells that may also express either activating or inhibitory receptors as described herein, one of the agents on the erythroid cell may comprise a targeting moiety, e.g., an antibody molecule that binds the T cell receptor (TCR) or another T cell marker. Targeting moieties are described in more detail in the section entitled “Localization configurations” herein. In some embodiments, a specific T cell subtype or clone may be enhanced or inhibited. In some embodiments, one or more of the agents on the erythroid cell is a peptide-MHC molecule that will selectively bind to a T cell receptor in an antigen-specific manner.

In some embodiments the engineered erythroid cell is used as a vaccine. In embodiments an engineered erythroid cell comprises a vaccine antigen and a polypeptide that induces activation of an APC. In embodiments an APC activating agents is an inflammatory cytokine or an activating receptor on T cells (see, e.g., Table 2), e.g. ICOS, CD28, CD40, 4-1BB, or OX40, or another polypeptide that binds APCs. In some embodiments, the erythroid cell further comprises an antibody molecule that binds a dendritic cell receptor, e.g. CD205 or CD206. In embodiments this agent promotes uptake by antigen presenting cells.

In some embodiments a plurality of agents comprise multiple antigens derived from a complex target, e.g., an infected eukaryotic cell, virus, or bacterium, against which it is desirable to mount a complex immune response with multiple specificities.

In some embodiments, the first and second exogenous polypeptides comprise, in some embodiments, an antigen and a costimulatory molecule, e.g., wherein the erythroid cell can act as an APC, e.g., for vaccination.

In some embodiments, an enucleated erythroid cell comprising a first exogenous polypeptide and a second exogenous polypeptide is administered to a subject having a first target and a second target. In embodiments, the first exogenous polypeptide acts on (e.g., binds) the first target and the second exogenous polypeptide acts on the second target. Optionally, the enucleated erythroid cell comprises a third exogenous polypeptide and the patient comprises a third target. In embodiments, the third exogenous polypeptide acts on the third target.

In some embodiments an erythroid cell comprises a first exogenous polypeptide which is an agonist or antagonist of a first target in a first pathway, and further comprises a second exogenous polypeptide which is an agonist or antagonist of a second target in a second pathway, wherein the first and second pathways act in concert toward a desired response. The first and second exogenous polypeptides can both be agonists; can both be antagonists; or one can be an agonist and the other can be an antagonist. In some embodiments, the target cell or tissue comprises a virus, bacterium, or infected tissue. In some embodiments, the erythroid cell further comprises a targeting agent.

Independent Function Configurations

When two or more agents (e.g., polypeptides) have an independent function relationship, the agents have two distinct (e.g., complementary) functions. For example, a first agent binds a first target and the second agent binds a second target. The patient may lack the first or second target. Optionally, the first and second agents are in different pathways.

In some embodiments the agents comprise a plurality of antibody molecules, which plurality of antibody molecules binds a plurality of common bacterial, fungal, or viral pathogens. In embodiments, the antibodies are generated from human B cells, e.g., a combinatorial library of pooled human B cells. In embodiments, the poly-antibody engineered erythroid cells serve as an alternative, replacement, or supplement to intravenous immunoglobulin (IVIG). IVIG is in embodiments administered to a patient having an infectious disease or at risk of contracting an infectious disease. The erythroid cell would provide long-lasting protection and be free from the risks associated with plasma donation.

In some embodiments, an enucleated erythroid cell comprising a first exogenous polypeptide and a second exogenous polypeptide is administered to a subject having a first target but not a second target, or wherein the patient is not known to have a first target or second target. In embodiments, the first exogenous polypeptide acts on (e.g., binds) the first target and the second exogenous polypeptide remains substantially unbound. Optionally, the enucleated erythroid cell comprises a third exogenous polypeptide and the patient lacks a third target, or is not known to have the third target. In some embodiments, the enucleated erythroid cell comprises a plurality of exogenous polypeptides, and the patient does not have, or is not known to have, targets for one or a subset of the plurality of exogenous polypeptides.

In some embodiments, one or more (e.g., two or more) of the targets are toxins (e.g., bacterial or fungal toxins or bioterror agents). In embodiments, the erythroid cell provides pan-bioterror-threat protection. In embodiments, the erythroid cell comprises a plurality of agents and protects (e.g., by capture, neutralization, and clearance) from a broad array of potential toxins as a single therapeutic agent. In some embodiments, the toxin comprises, e.g., anthrax toxin (e.g., PA, EF, or LF, or any combination thereof), a botulinum toxins (A, B, C, D, E, F, G, or any combination thereof), ricin, saxitoxin, Staphyloccocal enterotoxin B, Tetrodotoxin, or Trichothecene mycotoxins. In embodiments, the polypeptides comprise one or more antibody molecules.

In some embodiments the agents comprise a plurality of antibody molecules, which plurality of antibody molecules binds a plurality of common bacterial, fungal, or viral pathogens. In embodiments, the antibodies are generated from human B cells, e.g., a combinatorial library of pooled human B cells. In embodiments, the poly-antibody engineered erythroid cells serve as an alternative, replacement, or supplement to intravenous immunoglobulin (IVIG). The erythroid cell would provide long-lasting protection and be free from the risks associated with plasma donation.

Localization Configurations

When two or more agents (e.g., polypeptides) have a localization relationship, a first agent localizes the erythroid cell to a site of action that enhances the activity of the second or other agent or agents compared to their activity when not localized to the site of action (e.g., by binding of the first agent to its target, there is an increase in the local concentration of the second agent in the area of its target). In some embodiments one agent serves to target the erythroid cell to a site of action and one or more agents have a therapeutic effect. In an embodiment, binding of the first agent increases the activity of an entity, e.g., polypeptide, bound by the second agent. In an embodiment, the first agent binds to a substrate or product of the entity, e.g., polypeptide, bound by the second agent. The agent that localizes the erythroid cell may be, e.g., a ligand for a receptor on a target cell, or an antibody that binds a cell surface molecule on a target cell.

The erythroid cell can comprise one or more targeting agents. The targeting agent can be an exogenous polypeptide. In embodiments, an erythroid cell comprises two targeting agents, which may increase the specificity and/or affinity and/or avidity of the erythroid cell binding to its target, compared to an otherwise similar erythroid cell comprising only one of the targeting agents. The erythroid cell optionally further comprises an exogenous polypeptide with therapeutic activity, e.g., anti-viral or anti-bacterial activity. The exogenous polypeptide with therapeutic activity can comprise an enzyme, capture reagent, agonist, or antagonist.

As another example, an erythroid cell comprises a targeting agent that binds to inflamed tissue, e.g., infected tissue and also comprises an anti-viral or anti-bacterial molecule. In some embodiments, the targeting agent binds an inflammatory integrin, e.g., an avB3 integrin or an addressin such as MADCAM1. In some embodiments, the targeting agent comprises a lymphocyte homing receptor (e.g., CD34 or GLYCAM-1) or integrin-binding portion or variant thereof.

In some embodiments, the erythroid cell targets a cell in an inflamed tissue, e.g., infected tissue. For instance, the cell can comprise one or more targeting agents, e.g., exogenous polypeptides that bind surface markers of inflamed tissue. The targeting agent can be an exogenous polypeptide comprising, e.g., an anti-VCAM antibody molecule or an anti-E-selectin antibody molecule. In embodiments, an erythroid cell comprises two targeting agents, which may increase the specificity and/or affinity and/or avidity of the erythroid cell binding to its target, compared to an otherwise similar erythroid cell comprising only one of the targeting agents. In embodiments, the targeting moieties comprise: a surface exposed anti-VCAM antibody molecule and a surface exposed anti-E-selectin antibody molecule; a surface exposed alpha4Beta1 integrin or fragment or variant thereof and a surface exposed anti-E-selectin antibody molecule; or a surface exposed alphavbeta2 integrin or fragment or variant thereof and a surface exposed anti-E-selectin antibody molecule. The erythroid cell optionally further comprises an exogenous polypeptide with therapeutic activity, e.g., anti-viral or anti-bacterial activity. The exogenous polypeptide with therapeutic activity can comprise an enzyme, capture reagent, agonist, or antagonist.

In embodiments, the targeting moiety comprises a receptor or a fragment or variant thereof. In embodiments, the targeting moiety comprises an antibody molecule such as an scFv.

In embodiments, the first exogenous polypeptide can comprise a targeting agent and the second exogenous polypeptide can comprise an enzyme. For example, in some embodiments, the erythroid cell comprises a first polypeptide comprising a targeting agent that binds a cell and a second polypeptide that inhibits the cell, or a virus or bacterium in proximity to the cell. For instance, the targeting agent can comprise an anti-CD4 antibody which binds CD4 on the surface of a T cell, or an anti-MAdCAM-1 antibody molecule.

The first exogenous polypeptide can comprise a targeting agent and the second exogenous polypeptide can comprise an agonist of a target. For instance, in some embodiments, the first exogenous polypeptide comprises an anti-MAdCAM-1 antibody molecule, e.g., which can bind MAdCAM-1, e.g., on inflamed, e.g., infected tissue. In embodiments, the targeting agent comprises a receptor or fragment or variant thereof, an antibody molecule, a ligand or fragment or variant thereof, a cytokine or fragment or variant thereof. In embodiments, the second exogenous polypeptide comprises an attenuator, an activator, a cell-killing agent, or a cytotoxic molecule (e.g., a small molecule, protein, RNA e.g., antisense RNA, or TLR ligand). In embodiments, the second exogenous polypeptide is intracellular, e.g., not membrane associated, and in some embodiments, the second exogenous polypeptide is surface-exposed.

The therapeutic agent may have, e.g., an anti-pathogenic effect, of which there are several strategies. For example the therapeutic agent may be an enzyme, e.g., asparaginase, or a fragment or variant thereof, that degrades metabolites that are selectively required by the pathogen. In some embodiments, the erythroid cell comprising asparaginase further comprises a transporter protein, e.g., a protein that transports asparaginase, e.g., SN2 or SAT2.

The erythroid cell can comprise a targeting agent and a capture agent. In embodiments, the targeting agent comprises a ligand or a cytokine or fragment or variant thereof, or an antibody molecule, e.g., an scFv. In embodiments, the capture agent comprises a receptor or fragment or variant thereof, or an antibody molecule, e.g., an scFv. In embodiments, the target is a bacterium or virus.

Proximity-Based Configurations

When two or more agents (e.g., polypeptides) have a proximity-based relationship, the two agents function more strongly, e.g., exert a more pronounced effect, when they are in proximity to each other than when they are physically separate. In embodiments, the two agents are in proximity when they are directly binding to each other, when they are part of a complex (e.g., linked by a third agent), when they are present on the same cell membrane, or when they are present on the same subsection of a cell membrane (e.g., within a lipid raft, outside a lipid raft, or bound directly or indirectly to an intracellular structure such as a cytoskeleton component). In some embodiments, first polypeptide binds a first target molecule and the second polypeptide binds a second target molecule, and this binding causes the first target molecule and the second target molecule to move into closer proximity with each other, e.g., to bind each other. In some embodiments, the first and second target molecules are cell surface receptors on a target cells.

In an embodiment, an erythroid cell comprises an optional first exogenous polypeptide, a second exogenous polypeptide, and a third exogenous polypeptide. The second and third exogenous polypeptides can bind to different epitopes within the same polypeptide chain of a target, e.g., a viral factor or a bacterial factor. The second and third exogenous polypeptides, which are mounted on the erythrocyte, bind to the target with higher avidity than if the second and third exogenous polypeptides were free polypeptides. As examples, two or more exogenous polypeptides could bind different sites on the same target, wherein the target is a toxin (e.g., a bacterial toxin), or a pathogen (e.g., a bacterium, virus, or parasite). As an example, the first exogenous polypeptide comprises a targeting moiety.

Scaffold Configurations

When two or more agents (e.g., polypeptides) have a scaffold relationship, the agents bring two or more targets together, to increase the likelihood of the targets interacting with each other. In an embodiment the first and second agent are associated with each other (forming a scaffold) at the surface of the erythroid cell, e.g., two complexed polypeptides. In an embodiment, the erythroid cell comprises a bispecific antibody molecule, e.g., an antibody molecule that recognizes one or more (e.g., 2) proteins described herein, e.g., in any of Table 1, Table 2, Table 3, and Table 4.

The targets may comprise, e.g., proteins, cells, small molecules, or any combination thereof. In an embodiment, the first and second targets are proteins. In an embodiment, the first and second targets are cells.

As another example, a an erythroid cell brings an immune effector cell (e.g., T cell) and a pathogen (e.g., bacterium) in close proximity with one another to facilitate the killing of the pathogen by the immune effector cell. Thus, in some embodiments, the first polypeptide binds a site on a pathogen (e.g., a protein) and the second polypeptide binds a cell surface marker of an immune effector cell. The first and second polypeptides may comprise, e.g., antibody molecules. In some embodiments, the immune cell marker is CD3.

In some embodiments, the erythroid cell brings an immune effector cell into proximity with another immune cell, e.g., to promote antigen presentation (e.g., when one cell is an antigen presenting cell and the other cell is a T cell).

In some embodiments, an erythroid cell expresses an exogenous fusion polypeptide comprising a first antibody molecule domain and a second antibody molecule domain, wherein the exogenous polypeptide functions as a bispecific antibody, e.g., wherein the first antibody molecule domain binds a first target on a first cell and the second antibody molecule domain binds a second target on a second cell, e.g., a different cell type.

Multimer Configurations

When two or more agents (e.g., polypeptides) have a multimer configuration, the agents combine with each other, e.g., bind each other, to form a complex that has a function or activity on a target.

In an embodiment, the agents are subunits of a cell surface complex, e.g., MHCI, and a function is to bind a peptide. In an embodiment, the agents are subunits of MHCII, and a function is to bind a peptide. In an embodiment, the agents are subunits of a cell surface molecule, e.g., MHCI and a peptide, e.g., a peptide loaded on the MHCI molecule, and a function is to present the peptide. The peptide may comprise an antigen, e.g., a bacterial sequence, a viral sequence, or other pathogen sequence. In some embodiments, the antigen is about 8, 9, 10, 11, 12, 13, 14, 15, 16, 17, 18, 19, 20, 25, 30, or 35 amino acids in length. In an embodiment, the agents are subunits of a MHCII and a peptide, e.g., a peptide loaded on the MHCII molecule, and a function is to present the peptide. In one embodiment, the complex is a functional MHC I, the agents are MHC I (alpha chain 1-3) and beta-2 microglobulin. In one embodiment the complex is MHC II and the agents are MHC II alpha chain and MHC II beta chain. In some embodiments, the MHC molecule comprises human MHC class I or II, e.g., MHC II alpha subunit and MHC II beta subunit or a fusion molecule comprising both subunits or antigen-presenting fragments thereof. An erythroid cell with these two polypeptides is used, in some embodiments, for immune induction or antigen presentation, e.g., for vaccination. In some embodiments, the erythroid cell comprises a single protein that is a fusion between an MHC molecule and an antigen, e.g., a single-chain peptide-MHC construct. In some embodiments, a non-membrane tethered component of the complex, e.g. the peptide, or the beta-2 microglobulin, is assembled with another agent within the cell prior to trafficking to the surface, is secreted by the cell then captured on the surface by the membrane-tethered component of the multimer, or is added in a purified form to an engineered erythroid cell.

In some embodiments, an enucleated erythroid cell acts on a complement cascade. Some complement regulatory proteins act in concert as co-factors for one another, e.g. CFH and CD55 are co-factors for the enzymatic activity of CFI. In some embodiments, the agents comprise an enzymatic protein or domain, e.g., CFI, and a co-factor, e.g., CFH or CD55, that accelerates and enhances the activity of CFI on the target complement molecule.

In some embodiments the complex comprises multiple subdomains derived from different polypeptide chains, all of which must be expressed in order for the complex to be active.

Pathway Biology Configurations

When two or more agents (e.g., polypeptides) have a pathway biology relationship, the agents act on successive steps of a pathway, e.g., to modulate complex systems. As an example, the first exogenous polypeptide is an enzyme that converts a substrate into an intermediate, and the second exogenous polypeptide is an enzyme that converts the intermediate into a product. Co-localization of the enzymes on or in the same erythroid cell allow the reaction to take place faster than it would in solution. In some embodiments, one, two, or more, of the enzymes are not natively found in humans; this would enable new metabolic pathways to be introduced.

In some embodiments, the enzymes synthesize a beneficial product. In some embodiments, the enzymes break down a harmful substrate. In some embodiments, the first exogenous polypeptide produces a reaction product, e.g., an undesired reaction product, e.g., peroxide, and the second exogenous polypeptide breaks down the reaction product.

In some embodiments, the first exogenous polypeptide has a first K_{M} for a first substrate and the second exogenous polypeptide has a second K_{M} for a second substrate. In embodiments, the first K_{M} is less than 90%, 80%, 70%, 60%, 50%, 40%, 30%, 20%, or 10% of the second K_{M}. In embodiments, the first K_{M} is at least 90%, 80%, 70%, 60%, 50%, 40%, 30%, 20%, or 10% of the second Kₘ

Compensatory Configurations

When two or more agents (e.g., polypeptides) have a compensatory relationship, a first agent reduces an undesirable characteristic of a second agent. For example, in some embodiments, the second agent has a given level of immunogenicity, and the first agent reduces the immunogenicity, e.g., by negatively signaling immune cells (see Table 3), or by shielding an antigenic epitope of the second agent. In some embodiments, the second agent has a given half-life, and the first agent increases the half-life of the second agent. For example, the first agent can comprise a chaperone or fragment or variant thereof.

An enucleated erythroid cell can co-express a therapeutic protein and its inhibitor. The inhibitor can be released (e.g., cease binding the therapeutic but remain on the surface of the cell) at the desired location in the body, to activate the therapeutic protein.

For instance, in some embodiments, the erythroid cell comprises a first exogenous polypeptide with therapeutic activity (e.g., an antiviral or antibacterial molecule), a second exogenous polypeptide (e.g., an antibody that binds the antiviral or antibacterial molecule) that inhibits the first exogenous polypeptide. The second polypeptide may inhibit activity of the first exogenous polypeptide until the erythroid cell is at a desired location, e.g., at infected tissue, e.g., limiting off-target effects. The second exogenous polypeptide may comprise a variant of the target that the first exogenous polypeptide binds. For instance, the variant can be a weakly-binding variant that is competed away in the presence of the target. In embodiments, the Kd of the first exogenous polypeptide for the second exogenous polypeptide is at least 2, 3, 5, 10, 20, 50, or 100-fold greater than the Kd of the first exogenous polypeptide for its target. The erythroid cell optionally comprises a third exogenous polypeptide that comprises a targeting agent.

In some embodiments, the enucleated erythroid cell comprises a prodrug that becomes a drug at a desired site in a subject.

Enucleated Erythroid Cells Comprising Three or More Agents (e.g., Polypeptides)

In embodiments, an enucleated erythroid cell described herein comprises three or more, e.g., at least 4, 5, 10, 20, 50, 100, 200, 500, or 1000 agents. In embodiments, a population of erythroid cells described herein comprises three or more, e.g., at least 4, 5, 10, 20, 50, 100, 200, 500, 1000, 2000, or 5000 agents, e.g., wherein different erythroid cells in the population comprise different agents or wherein different erythroid cells in the population comprise different pluralities of agents. In embodiments, two or more (e.g., all) of the agents in the erythroid cell or population of erythroid cell have agent-additive, agent-synergistic, multiplicative, independent function, localization-based, proximity-dependent, scaffold-based, multimer-based, pathway-based, or compensatory activity.

In embodiments, the erythroid cell is produced by contacting an erythroid cell progenitor cell with a plurality of mRNAs encoding the agents.

Pan-Influenza Therapeutic. In an embodiment, an antiviral therapeutic, e.g., a pan-influenza antiviral therapeutic or a therapeutic for another viral infection described herein, comprises a plurality of modified erythroid cells. A nucleic acid library encoding one or more (1, 2, 5, 10, 20, 50, 100, 200, 500, 1000, 2000, or 5000 or more) viral antagonists or binders as membrane fusion proteins can be generated. This library can be introduced into erythroid cell progenitors as a mixture or individually, using methods such as electroporation, transfection or viral transduction. In one embodiment, the cells are subsequently grown in differentiation media until the desired level of maturity. The cells can be purified and formulated for injection into a subject. Once injected, the cells circulate, bind, and neutralize virus particles in the bloodstream. The resulting universal therapeutic could be universally potent against a plurality of viral strains, and obviate the need to know the strain one wishes to target a priori. Furthermore, through its ability to target multiple serotypes at once, such an approach may minimize the emergence of resistant strains.

Pan-Influenza Vaccine. In an embodiment, an antiviral vaccine, e.g., a pan-influenza vaccine or a vaccine for another viral infection described herein, comprises a plurality of modified cells. A nucleic acid library encoding one or more (e.g., 1, 2, 5, 10, 20, 50, 100, 200, 500, 1000, 2000, or 5000 or more) viral antigens would be generated using methods. This library can be introduced into erythroid cell progenitors as a mixture or individually, using methods such as electroporation, transfection or viral transduction. In one embodiment, the cells would subsequently be grown in differentiation media until the desired level of maturity. The cells can be purified and formulated for injection into a patient. Once injected, the cells elicit a broad immune response against the library of original antigens and prime the immune system for responding to secondary infections. In one embodiment, the cells are co-injected with an adjuvant, or express an adjuvant protein, to further stimulate the immune response. The resulting universal vaccine could be potent against a wide variety of viral strains, and obviate the need to know the strain one wishes to target a priori. Furthermore, through its ability to target multiple serotypes at once, such an approach may minimize the emergence of resistant strains.

Physical Characteristics of Enucleated Erythroid Cells

In some embodiments, the erythroid cells described herein have one or more (e.g., 2, 3, 4, or more) physical characteristics described herein, e.g., osmotic fragility, cell size, hemoglobin concentration, or phosphatidylserine content. While not wishing to be bound by theory, in some embodiments an enucleated erythroid cell that expresses an exogenous protein has physical characteristics that resemble a wild-type, untreated erythroid cell. In contrast, a hypotonically loaded erythroid cell sometimes displays aberrant physical characteristics such as increased osmotic fragility, altered cell size, reduced hemoglobin concentration, or increased phosphatidylserine levels on the outer leaflet of the cell membrane.

In some embodiments, the enucleated erythroid cell comprises an exogenous protein that was encoded by an exogenous nucleic acid that was not retained by the cell, has not been purified, or has not existed fully outside an erythroid cell. In some embodiments, the erythroid cell is in a composition that lacks a stabilizer.

Osmotic Fragility

In some embodiments, the enucleated erythroid cell exhibits substantially the same osmotic membrane fragility as an isolated, uncultured erythroid cell that does not comprise an exogenous polypeptide. In some embodiments, the population of enucleated erythroid cells has an osmotic fragility of less than 50% cell lysis at 0.3%, 0.35%, 0.4%, 0.45%, or 0.5% NaCl. Osmotic fragility can be assayed using the method of Example 59 of WO2015/073587, which is herein incorporated by reference in its entirety.

Cell Size

In some embodiments, the enucleated erythroid cell has approximately the diameter or volume as a wild-type, untreated erythroid cell.

In some embodiments, the population of erythroid cells has an average diameter of about 4, 5, 6, 7, or 8 microns, and optionally the standard deviation of the population is less than 1, 2, or 3 microns. In some embodiments, the one or more erythroid cell has a diameter of about 4-8, 5-7, or about 6 microns. In some embodiments, the diameter of the erythroid cell is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system.

In some embodiment the volume of the mean corpuscular volume of the erythroid cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In one embodiment the mean corpuscular volume of the erythroid cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In one embodiment the mean corpuscular volume of the erythroid cells is between 80-100, 100-200, 200-300, 300-400, or 400-500 femtoliters (fL). In some embodiments, a population of erythroid cells has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter.

Hemoglobin Concentration

In some embodiments, the enucleated erythroid cell has a hemoglobin content similar to a wild-type, untreated erythroid cell. In some embodiments, the erythroid cells comprise greater than 1%, 2%, 3%, 4%, 5%, 6%, 7%, 8%, 9% or greater than 10% fetal hemoglobin. In some embodiments, the erythroid cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of WO2015/073587, which is herein incorporated by reference in its entirety.

Phosphatidylserine Content

In some embodiments, the enucleated erythroid cell has approximately the same phosphatidylserine content on the outer leaflet of its cell membrane as a wild-type, untreated erythroid cell. Phosphatidylserine is predominantly on the inner leaflet of the cell membrane of wild-type, untreated erythroid cells, and hypotonic loading can cause the phosphatidylserine to distribute to the outer leaflet where it can trigger an immune response. In some embodiments, the population of erythroid cells comprises less than about 30, 25, 20, 15, 10, 9, 8, 6, 5, 4, 3, 2, or 1% of cells that are positive for Annexin V staining. Phosphatidylserine exposure is assessed, in some embodiments, by staining for Annexin-V-FITC, which binds preferentially to PS, and measuring FITC fluorescence by flow cytometry, e.g., using the method of Example 54 of WO2015/073587, which is herein incorporated by reference in its entirety.

Other Characteristics

In some embodiments, the population of erythroid cells comprises at least about 50%, 60%, 70%, 80%, 90%, or 95% (and optionally up to 90 or 100%) of cells that are positive for GPA. The presence of GPA is detected, in some embodiments, using FACS.

In some embodiments, the erythroid cells have a half-life of at least 0.5, 1, 2, 7, 14, 30, 45, or 90 days in a subject.

In some embodiments, a population of cells comprising erythroid cells comprises less than about 10, 5, 4, 3, 2, or 1% echinocytes.

In some embodiments, an erythroid cell is enucleated, e.g., a population of cells comprising erythroid cells used as a therapeutic preparation described herein is greater than 50%, 60%, 70%, 80%, 90% enucleated. In some embodiments, a cell, e.g., an erythroid cell, contains a nucleus that is non-functional, e.g., has been inactivated.

### Methods of Manufacturing Enucleated Erythroid Cells

Methods of manufacturing enucleated erythroid cells comprising (e.g., expressing) an exogenous agent (e.g., polypeptides) are described, e.g., in WO2015/073587 and WO2015/153102, each of which is incorporated by reference in its entirety.

In some embodiments, hematopoietic progenitor cells, e.g., CD34+hematopoietic progenitor cells, are contacted with a nucleic acid or nucleic acids encoding one or more exogenous polypeptides, and the cells are allowed to expand and differentiate in culture.

In some embodiments, the two or more polypeptides are encoded in a single nucleic acid, e.g. a single vector. In embodiments, the single vector has a separate promoter for each gene, has two proteins that are initially transcribed into a single polypeptide having a protease cleavage site in the middle, so that subsequent proteolytic processing yields two proteins, or any other suitable configuration. In some embodiments, the two or more polypeptides are encoded in two or more nucleic acids, e.g., each vector encodes one of the polypeptides.

The nucleic acid may be, e.g., DNA or RNA. A number of viruses may be used as gene transfer vehicles including retroviruses, Moloney murine leukemia virus (MMLV), adenovirus, adeno-associated virus (AAV), herpes simplex virus (HSV), lentiviruses such as human immunodeficiency virus 1 (HIV 1), and spumaviruses such as foamy viruses, for example.

In some embodiments, the cells are produced using sortagging, e.g., as described in WO2014/183071 or WO2014/183066, each of which is incorporated by reference in its entirety.

Erythroid cells described herein can also be produced using coupling reagents to link an agent (e.g., an exogenous polypeptide) to a cell. For instance, click chemistry can be used. Coupling reagents can be used to couple an agent to a cell, for example, when the agent is a complex or difficult to express agent, e.g., a polypeptide, e.g., a multimeric polypeptide; large polypeptide; agent derivatized in vitro, e.g., polypeptide; agent that may have toxicity to, or which are not expressed efficiently in, the erythroid cells.

Thus, in some embodiments, an erythroid cell described herein comprises many as, at least, more than, or about 5,000, 10,000, 50,000, 100,000, 200,000, 300,000, 400,000, 500,000 coupling reagents per cell. In some embodiments, the erythroid cells are made by a method comprising a) coupling a first coupling reagent to an erythroid cell, thereby making a pharmaceutical preparation, product, or intermediate. In an embodiment, the method further comprises: b) contacting the cell with an agent coupled to a second coupling reagent e.g., under conditions suitable for reaction of the first coupling reagent with the second coupling reagent. In embodiments, two or more agents are coupled to the cell (e.g., using click chemistry). In embodiments, a first agent is coupled to the cell (e.g., using click chemistry) and a second agent comprises a polypeptide expressed from an exogenous nucleic acid.

In some embodiments, the coupling reagent comprises an azide coupling reagent. In some embodiments, the azide coupling reagent comprises an azidoalkyl moiety, azidoaryl moiety, or an azidoheteroaryl moiety. Exemplary azide coupling reagents include 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. Coupling reagents may also comprise an alkene moiety, e.g., a transcycloalkene moiety, an oxanorbornadiene moiety, or a tetrazine moiety. Additional coupling reagents can be found in Click Chemistry Tools (https://clickchemistrytools.com/) or Lahann, J (ed) (2009) Click Chemistry for Biotechnology and Materials Science, each of which is incorporated herein by reference in its entirety.

In some embodiments, the erythroid cells are expanded at least 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, the population of erythroid cells comprises at least 30, 35, 40, 45, 50, 55, 60, 65, 70, 75, 80, 85, 90, 95, or 98% (and optionally up to about 80, 90, or 100%) enucleated erythroid cells. In some embodiments, the population of erythroid cells contains less than 1% live enucleated cells, e.g., contains no detectable live enucleated cells. Enucleation is measured, in some embodiments, by FACS using a nuclear stain. In some embodiments, at least 30, 35, 40, 45, 50, 55, 60, 65, 70, 75, or 80% (and optionally up to about 70, 80, 90, or 100%) of erythroid cells in the population comprise one or more (e.g., 2, 3, 4 or more) of the exogenous polypeptides. Expression of the polypeptides is measured, in some embodiments, by erythroid cells using labeled antibodies against the polypeptides. In some embodiments, at least 30, 35, 40, 45, 50, 55, 60, 65, 70, 75, or 80% (and optionally up to about 70, 80, 90, or 100%) of erythroid cells in the population are enucleated and comprise one or more (e.g., 2, 3, 4, or more) of the exogenous polypeptides. In some embodiments, the population of erythroid cells comprises about 1×10⁹-2×10⁹, 2×10⁹-5×10⁹, 5×10⁹-1×10¹⁰, 1×10¹°-2×10¹⁰, 2×10¹°-5×10¹⁰, 5×10¹°-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

### Physically Proximal, Synergistic Agents

In some aspects, the present disclosure provides a composition comprising a first agent and a second agent in physical proximity to each other. In some embodiments, agents act synergistically when they are in physical proximity to each other but not when they are separate. In some embodiments, the first and second agents are covalently linked, e.g., are part of a fusion protein or are chemically conjugated together. In some embodiments, the first and second agent are non-covalently linked, e.g., are bound directly to each other or to a scaffold. In some embodiments, the first and second agents are part of (e.g., linked to) a nanoparticle (e.g., 1-100, 100-2,500, or 2,500-10,000 nm in diameter) liposome, vesicle, bead, polymer, implant, or polypeptide complex.

In some embodiments, the composition comprises at least 3, 4, 5, 6, 7, 8, 9, or 10 different agents that are in physical proximity to each other (e.g., covalently or noncovalently linked).

In some embodiments, the composition comprises one or more (e.g., 2, 3, 4, 5, or more) agents described herein, e.g., exogenous polypeptides described herein, e.g., polypeptides of any of Table 1, Table 2, Table 3, or Table 4, or a fragment or variant thereof, or an antibody molecule thereto. In some embodiments, one or more (e.g., 2, 3, or more) of the exogenous polypeptides comprise a first polypeptide of Table 4 and a second polypeptide of Table 4.

### Engineered Erythroid Cells Comprising One or More Agents

In some aspects, the present disclosure provides an engineered erythroid cell comprising an exogenous agent. More specifically, in some aspects, the present disclosure provides an enucleated erythroid cell comprising an exogenous polypeptide. The erythroid cell optionally further comprises a second, different, exogenous polypeptide.

In some embodiments, the exogenous polypeptide (e.g., an exogenous polypeptide comprised by an enucleated erythroid cell that optionally further comprises a second exogenous polypeptide) is an exogenous polypeptide described herein. In embodiments, the polypeptide is selected from any of Table 1, Table 2, Table 3, or Table 4, or a fragment or variant thereof, or an antibody molecule thereto.

In some embodiments, the exogenous polypeptide comprises an antibacterial protein such as rBPI21, e.g., for the treatment of bacteremia or LPS toxicity.

### Vehicles for Polypeptides Described Herein

While in many embodiments herein, the one or more (e.g., two or more) exogenous polypeptides are situated on or in an enucleated erythroid cell, it is understood that any exogenous polypeptide or combination of exogenous polypeptides described herein can also be situated on or in another vehicle. The vehicle can comprise, e.g., a cell, an erythroid cell, a corpuscle, a nanoparticle, a micelle, a liposome, or an exosome. For instance, in some aspects, the present disclosure provides a vehicle (e.g., a cell, an erythroid cell, a corpuscle, a nanoparticle, a micelle, a liposome, or an exosome) comprising, e.g., on its surface, one or more agents described herein. In some embodiments, the one or more agents comprise an agent selected a polypeptide of any of Table 1, Table 2, Table 3, or Table 4, or a fragment or variant thereof, or an agonist or antagonist thereof, or an antibody molecule thereto. In some embodiments, the vehicle comprises two or more agents described herein, e.g., any pair of agents described herein.

In some embodiments, the vehicle comprises an erythroid cell. In embodiments, the erythroid cell is a nucleated red blood cell, red blood cell precursor, or enucleated red blood cell. In embodiments, the erythroid cell is a cord blood stem cell, a CD34+ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), a reticulocyte, an erythrocyte, an induced pluripotent stem cell (iPSC), a mesenchymal stem cell (MSC), a polychromatic normoblast, an orthochromatic normoblast, or a combination thereof. In some embodiments, the erythroid cells are immortal or immortalized cells.

### Heterogeneous Populations of Cells

While in many embodiments herein, the one or more (e.g., two or more) exogenous polypeptides are situated on or in a single cell, it is understood that any exogenous polypeptide or combination of exogenous polypeptides described herein can also be situated on a plurality of cells. For instance, in some aspects, the disclosure provides a plurality of erythroid cells, wherein a first cell of the plurality comprises a first agent (e.g., an exogenous polypeptide, e.g., an exogenous polypeptide described herein) and a second cell of the plurality comprises a second agent (e.g., an exogenous polypeptide, e.g., an exogenous polypeptide described herein). In some embodiments, the plurality of cells comprises two or more agents described herein, e.g., any pair of agents described herein. In some embodiments, less than 90%, 80%, 70%, 60%, 50%, 40%, 30%, 20%, 10%, 5%, 2%, or 1% of the cells in the population comprise both the first exogenous polypeptide and the second exogenous polypeptide.

### Cells Encapsulated in a Membrane

In some embodiments, enucleated erythroid cells or other vehicles described herein are encapsulated in a membrane, e.g., semi-permeable membrane. In embodiments, the membrane comprises a polysaccharide, e.g., an anionic polysaccharide alginate. In embodiments, the semipermeable membrane does not allow cells to pass through, but allows passage of small molecules or macromolecules, e.g., metabolites, proteins, or DNA. In embodiments, the membrane is one described in Lienert et al., “Synthetic biology in mammalian cells: next generation research tools and therapeutics” Nature Reviews Molecular Cell Biology 15, 95-107 (2014), incorporated herein by reference in its entirety. While not wishing to be bound by theory, in some embodiments, the membrane shields the cells from the immune system and/or keeps a plurality of cells in proximity, facilitating interaction with each other or each other's products.

Methods of Treatment with Compositions Herein, e.g., Enucleated Erythroid Cells

Methods of administering enucleated erythroid cells (e.g., reticulocytes) comprising (e.g., expressing) exogenous agent (e.g., polypeptides) are described, e.g., in WO2015/073587 and WO2015/153102, each of which is incorporated by reference in its entirety.

In embodiments, the enucleated erythroid cells described herein are administered to a subject, e.g., a mammal, e.g., a human. Exemplary mammals that can be treated include without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like). The methods described herein are applicable to both human therapy and veterinary applications.

In some embodiments, the erythroid cells are administered to a patient every 1, 2, 3, 4, 5, or 6 months.

In some embodiments, a dose of erythroid cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some embodiments, the erythroid cells are administered to a patient in a dosing regimen (dose and periodicity of administration) sufficient to maintain function of the administered erythroid cells in the bloodstream of the patient over a period of 2 weeks to a year, e.g., one month to one year or longer, e.g., at least 2 weeks, 4 weeks, 6 weeks, 8 weeks, 3 months, 6 months, a year, 2 years.

In some aspects, the present disclosure provides a method of treating a disease or condition described herein, comprising administering to a subject in need thereof a composition described herein, e.g., an enucleated erythroid cell described herein. In some embodiments, the disease or condition is an infectious disease. In some aspects, the disclosure provides a use of an erythroid cell described herein for treating a disease or condition described herein, e.g., an infectious disease. In some aspects, the disclosure provides a use of an erythroid cell described herein for manufacture of a medicament for treating a disease or condition described herein, e.g., an infectious disease.

An enucleated erythroid cell population may comprise, for example, a plurality (e.g., 2, 3, 4, or 5 or more) of infectious disease polypeptides. The enucleated erythroid cell populations comprising infectious disease polypeptides may be formulated in a pharmaceutical composition comprising an appropriate excipient (e.g., AS-3 additive solution) and administered, e.g., intravenously, to a patient, e.g., a patient suffering from or at risk of contracting an infectious disease.

Viral infections include adenovirus, coxsackievirus, hepatitis A virus, poliovirus, Epstein-Barr virus, herpes simplex type 1, herpes simplex type 2, human cytomegalovirus, human herpesvirus type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

### Additional Tables

TABLE 4
Amino acid sequences of infectious disease
therapeutic polypeptides described herein

| Seq | Sequence | | |
|---|---|---|---|
| ID | Name | ProtID | Amino acid sequence |
| 1 | 4-1BB-L | Uniprot | ACPWAVSGARASPGSAASPRLREGPELSPDDPAGLLDL |
| | | P41273 | RQGMFAQLVAQNVLLIDGPLSWYSDPGLAGVSLTGGLS |
| | | | YKEDTKELVVAKAGVYYVFFQLELRRVVAGEGSGSVS |
| | | | LALHLQPLRSAAGAAALALTVDLPPASSEARNSAFGFQ |
| | | | GRLLHLSAGQRLGVHLHTEARARHAWQLTQGATVLGL |
| | | | FRVTPEIPAGLPSPRSE |
| 2 | Anti PD-L1 | | VQLVESGGGLVQPGGSLRLSCAASGFTFSDSWIHWVRQ |
| | scFv | | APGKGLEWVAWISPYGGSTYYADSVKGRFTISADTSKN |
| | | | TAYLQMNSLRAEDTAVYYCARRHWPGGFDYWGQGTL |
| | | | VTVSSGGGGSGGGGSGGGGSIQMTQSPSSLSASVGDRV |
| | | | TITCRASQDVSTAVAWYQQKPGKAPKLLIYSASFLYSG |
| | | | VPSRFSGSGSGTDFTLTISSLQPEDFATYYCQQYLYHPA |
| | | | TFGQGTKVEIK |
| 3 | Linker | | GGGSGGGSGGGS |
| 4 | HA peptide | | YPYDVPDY |
| 5 | Flag peptide | | DYKDDDDK |
| 6 | ICOS-L | Uniprot | DTQEKEVRAMVGSDVELSCACPEGSRFDLNDVYVYWQ |
| | | O75144 | TSESKTVVTYHIPQNSSLENVDSRYRNRALMSPAGMLR |
| | | | GDFSLRLFNVTPQDEQKFHCLVLSQSLGFQEVLSVEVTL |
| | | | HVAANFSVPVVSAPHSPSQDELTFTCTSINGYPRPNVYW |
| | | | INKTDNSLLDQALQNDTVFLNMRGLYDVVSVLRIARTP |
| | | | SVNIGCCIENVLLQQNLTVGSQTGNDIGERDKITENPVS |
| | | | TGEKNAAT |
| 7 | OX40-L | Uniprot | QVSHRYPRIQSIKVQFTEYKKEKGFILTSQKEDEIMKVQ |
| | | P23510 | NNSVIINCDGFYLISLKGYFSQEVNISLHYQKDEEPLFQL |
| | | | KKVRSVNSLMVASLTYKDKVYLNVTTDNTSLDDFHVN |
| | | | GGELILIHQNPGEFCVL |
| 8 | GITR-L | Uniprot | QLETAKEPCMAKFGPLPSKWQMASSEPPCVNKVSDWK |
| | | Q9UNG2 | LEILQNGLYLIYGQVAPNANYNDVAPFEVRLYKNKDMI |
| | | | QTLTNKSKIQNVGGTYELHVGDTIDLIFNSEHQVLKNNT |
| | | | YWGIILLANPQFIS |
| 9 | Erwinia | Uniprot | MERWFKSLFV LVLFFVFTAS AADKLPNIVI |
| | chrysanthemi | P06608 | LATGGTIAGS AATGTQTTGY KAGALGVDTL |
| | asparaginase | | INAVPEVKKL ANVKGEQFSN MASENMTGDV |
| | (EcAspara- | | VLKLSQRVNELLARDDVDGV VITHGTDTVE |
| | ginase) | | ESAYFLHLTV KSDKPVVFVA AMRPATAISA |
| | | | DGPMNLLEAV RVAGDKQSRG RGVMVVLNDR |
| | | | IGSARYITKT NASTLDTFKA NEEGYLGVII |
| | | | GNRIYYQNRI DKLHTTRSVF DVRGLTSLPK |
| | | | VDILYGYQDD PEYLYDAAIQ HGVKGIVYAG |
| | | | MGAGSVSVRG IAGMRKAMEK GVVVIRSTRT |
| | | | GNGIVPPDEE LPGLVSDSLN PAHARILLML |
| | | | ALTRTSDPKV IQEYFHTY |
| 10 | Human SN2 | Uniprot | MELQDPKMNG ALPSDAVGYR QEREGFLPSR |
| | | Q8WUX | GPAPGSKPVQ FMDFEGKTSF GMSVFNLSNA |
| | | 1 | IMGSGILGLA YAMAHTGVIF FLALLLCIAL |
| | | | LSSYSIHLLL TCAGIAGIRA YEQLGQRAFG |
| | | | PAGKVVVATV ICLHNVGAMS SYLFIIKSEL |
| | | | PLVIGTFLYM DPEGDWFLKG NLLIIIVSVL |
| | | | IILPLALMKH LGYLGYTSGL SLTCMLFFLV |
| | | | SVIYKKFQLG CAIGHNETAM ESEALVGLPS |
| | | | QGLNSSCEAQ MFTVDSQMSY TVPIMAFAFV |
| | | | CHPEVLPIYT ELCRPSKRRM QAVANVSIGA |
| | | | MFCMYGLTAT FGYLTFYSSV KAEMLHMYSQ |
| | | | KDPLILCVRL AVLLAVTLTV PVVLFPIRRA |
| | | | LQQLLFPGKA FSWPRHVAIA LILLVLVNVL |
| | | | VICVPTIRDI FGVIGSTSAP SLIFILPSIF YLRIVPSEVE |
| | | | PFLSWPKIQA LCFGVLGVLF MAVSLGFMFA |
| | | | NWATGQSRMS GH |
| 11 | Human | Uniprot | MKKAEMGRFS ISPDEDSSSY SSNSDFNYSY |
| | SAT2 | Q96QD8 | PTKQAALKSH YADVDPENQN FLLESNLGKK |
| | | | KYETEFHPGT TSFGMSVFNL SNAIVGSGIL |
| | | | GLSYAMANTG IALFIILLTF VSIFSLYSVH |
| | | | LLLKTANEGG SLLYEQLGYK AFGLVGKLAA |
| | | | SGSITMQNIG AMSSYLFIVK YELPLVIQAL |
| | | | TNIEDKTGLW YLNGNYLVLL |
| | | | VSLVVILPLS LFRNLGYLGY TSGLSLLCMV |
| | | | FFLIVVICKK FQVPCPVEAA LIINETINTT LTQPTALVPA |
| | | | LSHNVTENDS CRPHYFIFNS QTVYAVPILI |
| | | | FSFVCHPAVL PIYEELKDRS RRRMMNVSKI |
| | | | SFFAMFLMYL LAALFGYLTF YEHVESELLH |
| | | | TYSSILGTDI LLLIVRLAVL MAVTLTVPVV IFPIRSSVTH |
| | | | LLCASKDFSW WRHSLITVSI LAFTNLLVIF VPTIRDIFGF |
| | | | IGASAASMLI FILPSAFYIK LVKKEPMKSV |
| | | | QKIGALFFLL SGVLVMTGSM ALIVLDWVHN PGGGH |
| 12 | Human | Uniprot | TTPEPCELDDEDFRCVCNFSEPQPDWSEAFQCVSAVEVE |
| | CD14 | P08571 | IHAGGLNLEPFLKRVDADADPRQYADTVKALRVRRLT |
| | | | VGAAQVPAQLLVGALRVLAYSRLKELTLEDLKITGTMP |
| | | | PLPLEATGLALSSLRLRNVSWATGRSWLAELQQWLKPG |
| | | | LKVLSIAQAHSPAFSCEQVRAFPALTSLDLSDNPGLGER |
| | | | GLMAALCPHKFPAIQNLALRNTGMETPTGVCAALAAA |
| | | | GVQPHSLDLSHNSLRATVNPSAPRCMWSSALNSLNLSF |
| | | | AGLEQVPKGLPAKLRVLDLSCNRLNRAPQPDELPEVDN |
| | | | LTLDGNPFLVPGTALPHEGSMN |
| 13 | Human | Uniprot | ESWEPCVEVVPNITYQCMELNFYKIPDNLPFSTKNLDLS |
| | TLR4 | O00206 | FNPLRHLGSYSFFSFPELQVLDLSRCEIQTIEDGAYQSLS |
| | | | HLSTLILTGNPIQSLALGAFSGLSSLQKLVAVETNLASLE |
| | | | NFPIGHLKTLKELNVAHNLIQSFKLPEYFSNLTNLEHLD |
| | | | LSSNKIQSIYCTDLRVLHQMPLLNLSLDLSLNPMNFIQPG |
| | | | AFKEIRLHKLTLRNNFDSLNVMKTCIQGLAGLEVHRLV |
| | | | LGEFRNEGNLEKFDKSALEGLCNLTIEEFRLAYLDYYLD |
| | | | DIIDLFNCLTNVSSFSLVSVTIERVKDFSYNFGWQHLELV |
| | | | NCKFGQFPTLKLKSLKRLTFTSNKGGNAFSEVDLPSLEF |
| | | | LDLSRNGLSFKGCCSQSDFGTTSLKYLDLSFNGVITMSS |
| | | | NFLGLEQLEHLDFQHSNLKQMSEFSVFLSLRNLIYLDIS |
| | | | HTHTRVAFNGIFNGLSSLEVLKMAGNSFQENFLPDIFTE |
| | | | LRNLTFLDLSQCQLEQLSPTAFNSLSSLQVLNMSHNNFF |
| | | | SLDTFPYKCLNSLQVLDYSLNHIMTSKKQELQHFPSSLA |
| | | | FLNLTQNDFACTCEHQSFLQWIKDQRQLLVEVERMECA |
| | | | TPSDKQGMPVLSLNITCQMNK |
| 14 | Human | Uniprot | ESSNQASLSCDRNGICKGSSGSLNSIPSGLTEAVKSLDLS |
| | TLR2 | O60603 | NNRITYISNSDLQRCVNLQALVLTSNGINTIEEDSFSSLG |
| | | | SLEHLDLSYNYLSNLSSSWFKPLSSLTFLNLLGNPYKTL |
| | | | GETSLFSHLTKLQILRVGNMDTFTKIQRKDFAGLTFLEE |
| | | | LEIDASDLQSYEPKSLKSIQNVSHLILHMKQHILLLEIFV |
| | | | DVTSSVECLELRDTDLDTFHFSELSTGETNSLIKKFTFRN |
| | | | VKITDESLFQVMKLLNQISGLLELEFDDCTLNGVGNFRA |
| | | | SDNDRVIDPGKVETLTIRRLHIPRFYLFYDLSTLYSLTER |
| | | | VKRITVENSKVFLVPCLLSQHLKSLEYLDLSENLMVEEY |
| | | | LKNSACEDAWPSLQTLILRQNHLASLEKTGETLLTLKNL |
| | | | TNIDISKNSFHSMPETCQWPEKMKYLNLSSTRIHSVTGCI |
| | | | PKTLEILDVSNNNLNLFSLNLPQLKELYISRNKLMTLPD |
| | | | ASLLPMLLVLKISRNAITTFSKEQLDSFHTLKTLEAGGN |
| | | | NFICSCEFLSFTQEQQALAKVLIDWPANYLCDSPSHVRG |
| | | | QQVQDVRLSVSECHRT |
| 15 | Human CD4 | Uniprot | KKVVLGKKGDTVELTCTASQKKSIQFHWKNSNQIKILG |
| | | P01730 | NQGSFLTKGPSKLNDRADSRRSLWDQGNFPLIIKNLKIE |
| | | | DSDTYICEVEDQKEEVQLLVFGLTANSDTHLLQGQSLT |
| | | | LTLESPPGSSPSVQCRSPRGKNIQGGKTLSVSQLELQDSG |
| | | | TWTCTVLQNQKKVEFKIDIVVLAFQKASSIVYKKEGEQ |
| | | | VEFSFPLAFTVEKLTGSGELWWQAERASSSKSWITFDLK |
| | | | NKEVSVKRVTQDPKLQMGKKLPLHLTLPQALPQYAGS |
| | | | GNLTLALEAKTGKLHQEVNLVVMRATQLQKNLTCEV |
| | | | WGPTSPKLMLSLKLENKEAKVSKREKAVWVLNPEAGM |
| | | | WQCLLSDSGQVLLESNIKVLPTWSTPVQP |
| 16 | Human | Uniprot | DYQVSSPIYDINYYTSE |
| | CCR5p18 | P51681 | |
| 17 | Human | Uniprot | DYQVSSPIYDINYYTSEPCQKINVKQIAA |
| | CCR5p30 | P51681 | |
| 18 | anti-IgE | | EVQLVESGGGLVQPGGSLRLSCAVSGYSITSGYSWNWI |
| | | | RQAPGKGLEWVASITYDGSTNYNPSVKGRITISRDDSKN |
| | | | TFYLQMNSLRAEDTAVYYCARGSHYFGHWHFAVWGQ |
| | | | GTLVTVSSGGGGSGGGGSGGGGSDIQLTQSPSSLSASVG |
| | | | DRVTITCRASQSVDYDGDSYMNWYQQKPGKAPKLLIY |
| | | | AASYLESGVPSRFSGSGSGTDFTLTISSLQPEDFATYYCQ |
| | | | QSHEDPYTFGQGTKVEIK |
| 19 | Anti-TNFa | | EVQLVESGGGLVQPGRSLRLSCAASGFTFDDYAMHWV |
| | scFv | | RQAPGKGLEWVSAITWNSGHIDYADSVEGRFTISRDNA |
| | | | KNSLYLQMNSLRAEDTAVYYCAKVSYLSTASSLDYWG |
| | | | QGTLVTVSSGGGGSGGGGSGGGGSDIQMTQSPSSLSAS |
| | | | VGDRVTITCRASQGIRNYLAWYQQKPGKAPKLLIYAAS |
| | | | TLQSGVPSRFSGSGSGTDFTLTISSLQPEDVATYYCQRY |
| | | | NRAPYTFGQGTKVEIK |
| 20 | anti-PCSK9 | | EVQLVQSGAEVKKPGASVKVSCKASGYTLTSYGISWVR |
| | | | QAPGQGLEWMGWVSFYNGNTNYAQKLQGRGTMTTDP |
| | | | STSTAYMELRSLRSDDTAVYYCARGYGMDVWGQGTT |
| | | | VTVSSGGGGSGGGGSGGGGSESALTQPASVSGSPGQSIT |
| | | | ISCTGTSSDVGGYNSVSWYQQHPGKAPKLMIYEVSNRP |
| | | | SGVSNRFSGSKSGNTASLTISGLQAEDEADYYCNSYTST |
| | | | SMVFGGGTKLTVL |
| 21 | anti-IL6 R | | QVQLQESGPGLVRPSQTLSLTCTVSGYSITSDHAWSWV |
| | scFV | | RQPPGRGLEWIGYISYSGITTYNPSLKSRVTMLRDTSKN |
| | | | QFSLRLSSVTAADTAVYYCARSLARTTAMDYWGQGSL |
| | | | VTVSSGGGGSGGGGSGGGGSDIQMTQSPSSLSASVGDR |
| | | | VTITCRASQDISSYLNWYQQKPGKAPKLLIYYTSRLHSG |
| | | | VPSRFSGSGSGTDFTFTISSLQPEDIATYYCQQGNTLPYT |
| | | | FGQGTKVEIKR |

TABLE 5
Exemplary modifiers, e.g., proteases

| | |
|---|---|
| Modifier | Exemplary target |

Proteases

| | |
|---|---|
| IdeS | IgG |
| IdeZ (an immunoglobulin-degrading | IgG |
| enzyme from Streptococcus equi | |
| subspecies zooepidemicus) | |
| IgA protease | IgG |
| Papain | IgG |
| ADAM17/TACE | TNF-alpha |
| mesotrypsin | Peptides comprising linkages |
| | involving the carboxyl group |
| | of lysine or arginine |
| Lysozyme | peptidoglycan |
| Endolysin | peptidoglycan |
| Endoproteinase, e.g., LysC (can cleave | Protein having a Lys-Xaa |
| proteins on C-terminal side of | motif |
| lysine residues) | |
| Metalloendopeptidase, e.g., LysNe (can cleave | Protein having an Xaa-Lys |
| proteins on amino side of lysine residues) | motif |
| Elastase, e.g., Pseudomonas elastase (PaE) | C3 |
| alkaline protease (PaAP) | C3 |
| 56 kDa protease from Serratia marcescens | C5a, C1-INH, alpha |
| | 2-antiplasmin, |
| | antithrombin III |
| C5a peptidase, e.g., Streptocoocal C5a | C5a |
| peptidase, ScpA, ScpB, SCPA | |
| Plasmin | IgG, C3b, iC3b |
| cysteine protease, e.g., Streptococcal | IgG, cytokines, extracellular |
| pyrogenic exotoxin B (SpeB) | matrix proteins |
| PrtH (e.g., from Porphyromonas) | IgG or C3 |
| Staphylokinase | plasminogen, IgG, C3b |
| Matrix metalloproteinases (e.g., | ECM proteins, e.g., collagen, |
| MMP1, MMP2, MMP3, MMP7, | gelatin, fibronectin, laminin, |
| MMP8, MMP9, MMP10, | aggrecan, elastin, fibrin |
| MMP11, MMP12, MMP13, | |
| MMP14, MMP15, MMP16, | |
| MMP17, MMP19, MMP20, | |
| MMP21, MMP23A, | |
| MMP23B, MMP24, MMP25, | |
| MMP26, MMP27, MMP28) | |

Other modifiers

| | |
|---|---|
| Protein disulfide isomerases | Proteins comprising two |
| | cysteine residues |
| Glycosyltransferases, e.g., α-glucan- | Protein comprising tyrosine, |
| branching glycosyltransferase, enzymatic | serine, threonine, or |
| branching factor, branching | asparagine glycosylation |
| glycosyltransferase, enzyme Q, | site |
| glucosan transglycosylase, glycogen | |
| branching enzyme, amylose | |
| isomerase, plant branching enzyme, | |
| α-1,4-glucan: α-1,4-glucan-6-, | |
| glycosyltransferase, | |
| starch branching enzyme, | |
| UDP-N-acetyl-D-galactosamine, | |
| polypeptide, | |
| N-acetylgalactosaminyltransferase, | |
| GDP-fucose protein | |
| O-fucosyltransferase 2, O-GlcNAc | |
| transferase | |
| Acetyltransferases or deacetylases, e.g., | histone |
| nucleosome-histone acetyltransferase, histone | |
| acetokinase, histone acetylase, histone | |
| transacetylase, histone deacetylase | |
| Acyltransferases | Protein comprising an acyl |
| | group |
| Phosphatases, e.g., protein-tyrosine- | phosphoprotein |
| phosphatase, phosphotyrosine phosphatase, | |
| phosphoprotein phosphatase | |
| (phosphotyrosine), phosphotyrosine | |
| histone phosphatase, protein phosphotyrosine | |
| phosphatase, tyrosylprotein phosphatase, | |
| phosphotyrosine protein phosphatase, | |
| phosphotyrosylprotein phosphatase, tyrosine | |
| O-phosphate phosphatase, PPT-phosphatase, | |
| PTPase, [phosphotyrosine] protein | |
| phosphatase, PTP-phosphatase | |
| Kinases, e.g., non-specific serine/threonine | Protein comprising a serine or |
| protein kinase, Fas-activated serine/threonine | threonine phosphorylation site |
| kinase, Goodpasture antigen-binding | |
| protein kinase, IκB kinase, | |
| cAMP-dependent protein kinase, | |
| cGMP-dependent protein kinase, protein | |
| kinase C, polo kinase, cyclin-dependent | |
| kinase, mitogen-activated | |
| protein kinase, mitogen-activated | |
| protein kinase kinase kinase, receptor | |
| protein serine/threonine kinase, | |
| dual-specificity kinase | |
| Gamma-carboxylases | Protein comprising glutamic acid |
| Methyltransferases | Protein comprising a lysine |
| | methylation site; DNA; RNA |
| Complement-factor inactivating moiety, | Complement factor, e.g., C1, C2a, |
| e.g., complement control protein, Factor | C4b, C3, C3a, C3b, C5, C5a, |
| H or Factor I | C5b, C6, C7, C8, or C9 |

TABLE 6
Exemplary first exogenous polypeptides that interact with a target

| Polypeptide | Exemplary target(s) |
|---|---|
| CD16A | IgG Fc |
| CD14 | LPS, e.g., bacterial LPS |
| TLR4 | LPS, e.g., bacterial LPS |
| scFv | Complement (e.g., C3 or C5) or |
| | cytokine (e.g., TNF-alpha or IL-6 |
| | or another cytokine of Table 1), |
| | viral protein (e.g., Hepatitis B |
| | surface antigen or HIV antigen) |
| Aquaporin 4 (AQP4) and variants | Anti-AQP4 autoantibodies |
| Phospholipase A2 receptor | Anti-PLA2R autoantibodies |
| (PLA2R) and variants (shortened | |
| domains & peptides) | |
| Acetylcholine receptor (AChR) and | Anti-AChR autoantibodies |
| variants (shortened | |
| domains & peptides) | |
| Insulin and variants (proinsulin, | Anti-insulin autoantibodies |
| preproinsulin, etc.) | |
| B2-glycoprotein 1 (b2GP1) and | Anti-b2GP1 autoantibodies |
| variants (shortened domains | |
| & peptides) | |
| ADAMTS13 and variants (shortened | Anti-ADAMTS13 autoantibodies |
| domains & peptides) | |
| GAD65 and variants (shortened | Anti-GAD65 autoantibodies |
| domains & peptides) | |
| Desmogleins, e.g., Desmoglein-3 or | Anti-Desmoglein autoantibodies |
| Desmoglein-1 and variants (shortened | |
| domains & peptides) | |
| Complement-factor binding moiety, | Complement factor, e.g., C1, C2a, |
| e.g., CD55 or CD46 | C4b, C3, C3a, C3b, C5, C5a, |
| | C5b, C6, C7, C8, or C9 |

### EXAMPLES

### Example 1

### Agent-Synergistic Activity of Enucleated Erythroid Cells Expressing Two Different TRAIL Receptor Ligands on the Surface

This Example describes the construction of enucleated erythroid cells comprising pro-apoptotic polypeptides. This type of cell can be used, e.g., to promote apoptosis of infected cells.

The genes for TRAIL receptor agonists DR4.2 (SEQ ID NO: 44) and DR5.2 (SEQ ID NO: 47) were synthesized. The genes were cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)).

Human CD34+ cells derived from mobilized peripheral blood cells from normal human donors were purchased frozen from AllCells Inc. Cells were thawed in PBS with 1% FBS. Cells were then cultured in StemSpan SFEM media with StemSpan CC100 Cytokine Mix at a density of 1E5 cells/mL. Media was swapped to differentiation media on day 5. Virus production protocol was conducted as follows. Briefly, HEK293T cells were seeded 24 hours before transfection. Cells were transfected with lentivector containing the construct along with packaging plasmids. A media swap was performed 24 hours after transfection and viruses were harvested 72 hours after transfection. On day 6 after thaw, cells were transduced with equal volumes of each virus in a 1:1 cell volume to virus volume ratio, and spinoculated at 845 xg for 1.5 hours with 5-10 μg/ml of polybrene.

Transduced cells were differentiated in defined media to erythroid lineage cells and to mature enucleated reticulocytes following the method of Hu et al., Blood 18 Apr. 2013 Vol 121, 16. In brief, the cell culture procedure was comprised of 3 phases. Composition of the base culture medium was Iscove's Modified Dulbecco's Medium, 2% human peripheral blood plasma, 3% human AB serum, 200 mg/mL Holohuman transferrin, 3 IU/mL heparin, and 10 mg/mL insulin. In the first phase (day 0 to day 6), CD34+ cells at a concentration of 10̂5/mL were cultured in the presence of 10 ng/mL stem cell factor, 1 ng/mL IL-3, and 3 IU/mL erythropoietin. In the second phase (day 7 to day 11), IL-3 was omitted from the culture medium. In the third phase that lasted until day 21, the cell concentration was adjusted to 10-6/mL on day 11 and to 5×10̂6/mL on day 15, respectively. The medium for this phase was the base medium plus 3 IU/mL erythropoietin, and the concentration of transferrin was adjusted to 1 mg/mL.

Expression of the transgenes was monitored by labeling with soluble TRAIL R1 and TRAIL R2 (purchased from Sigma-Aldrich Inc.) that had been chemically conjugated to complementary fluorescent dyes Fluorescein and DyLight 650 and staining by flow cytometry. Expression levels of both ligands DR4.2 and DR5.2 were verified through flow cytometry.

An apoptosis assay was conducted according to a modified version of Marconi et al., Cell Death and Disease 2013. In short, fully mature enucleated reticulocytes expressing DR4.2 and DR5.2 were incubated with CFSE-labeled Raji Cells for 24 hours at a 1:1 ratio. Afterwards cells were stained with annexin V and analyzed by flow cytometry. Apoptosis percentages were determined from CFSE positive Raji cells that also stained positive for annexin V.

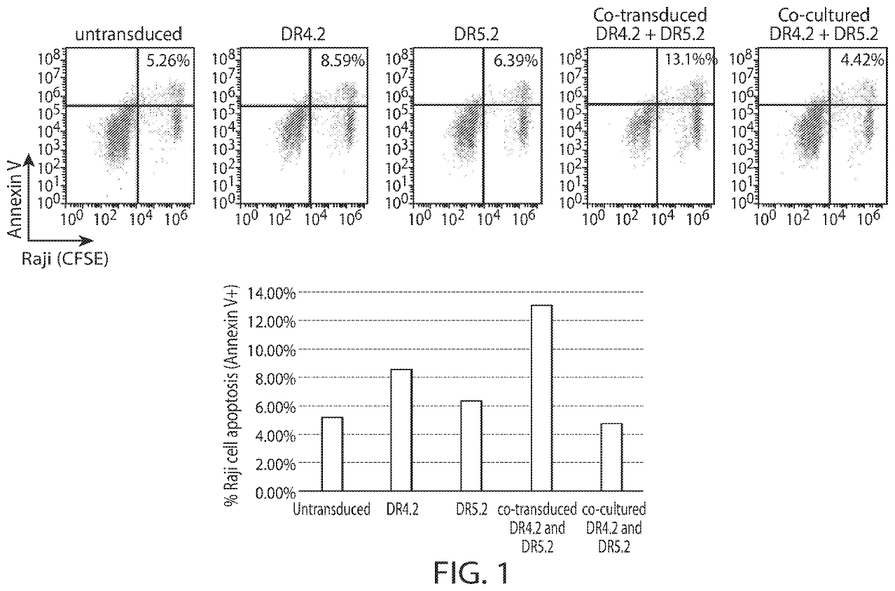

As shown in FIG. 1, when CFSE-labeled Raji cells were incubated with untransduced, DR4.2 transduced, DR5.2 transduced, or a mixture of DR4.2 transduced and DR5.2 transduced cultured reticulocytes, minimal cell death was observed over background. However, when CFSE-labeled Raji cells were incubated with cultured reticulocytes that had been simultaneously transduced with both DR4.2 and DR5.2 and thus express both proteins simultaneously, a significant amount of cell death was observed (equivalent to the maximal amount of TRAIL-induced apoptosis achievable in this assay with Raji cells—see, e.g. Marconi et al., Cell Death and Disease 2013). This data indicates that the coordinated action of TRAIL receptor agonists on the surface of a single engineered erythroid cell is able to induce cell killing in a synergistic manner, relative to cells expressing single TRAIL receptor agonists and even a mixture of cells that each express a different TRAIL receptor agonist.

### Example 2

### Generation and Activity of Enucleated Erythroid Cells Comprising Anti-PD-1, Anti-PD-L1, or Anti-CTLA4 as Infectious Disease Therapeutics

Enucleated erythroid cells were generated that express on their surface antibodies against PD-1 and PD-L1 (enucleated erythroid cells-antiPD-1 and enucleated erythroid cells-antiPD-L1) to assess whether these cells could bind their respective targets and activate a robust immune response. Binding of enucleated erythroid cells-antiPD-1 and enucleated erythroid cells-antiPD-L1 to recombinant PD-1 and PD-L1, respectively, was determined using flow cytometry, and was shown to be highly specific. Enucleated erythroid cells were also produced which express on their surface a fusion protein comprising, from N-to-C terminus, an ipilimumab-based anti-CTLA4 scFv antibody domain, an epitope tag, and full-length GPA (extracellular, transmembrane, and cytoplasmic domains). Robust expression of anti-CTLA4 polypeptides was observed in a flow cytometry assay, with over 95.2% of cells expressing anti-CTLA4 after transfection with a vector encoding anti-CTLA4.

Functional activity was tested using an in vitro Jurkat cell IL-2 secretion assay. In this assay, IL-2 secretion is inhibited by incubating Jurkat cells with NHL cells (Z138) expressing PD-L1 induced by stimulation with CD3/CD28 tetramers. IL-2 secretion was rescued by culturing the Jurkat and Z138 cells with enucleated erythroid cells-antiPD-1 or enucleated erythroid cells-antiPD-L1, but not control enucleated erythroid cells. Enucleated erythroid cells-antiCTLA4 also showed a rescue and restoration of T cell IL-2 secretion.

The ability of these engineered enucleated erythroid cells to elicit activation in a standard antigen recall assay was assayed. A robust 4-6 fold increase was demonstrated in interferon-gamma secretion of peripheral blood mononuclear cells (PBMC) in an antigen recall assay. Donor PBMC was stimulated with a common flu virus antigen. Memory T cells sensitive to immune checkpoint inhibition were tested for activation and gamma interferon secretion by co-culture with enucleated erythroid cells-antiPD-1 or enucleated erythroid cells-antiPD-L1 in comparison to control PBMCs or control enucleated erythroid cells.

These experiments indicate that enucleated erythroid cells are capable of engaging in specific cell-cell interactions and engaging the immune checkpoint.

### Example 3

### Enucleated Erythroid Cells Comprising Anti-PD-L1 Promotes T Cell Proliferation for Use in Treating an Infectious Disease

This Example demonstrates that co-culture of enucleated erythroid cells-antiPD-Llwith PBMC has led to enhanced T-cell proliferation, based upon a 4.4 fold increase in total count of T cells following incubation with enucleated erythroid cells-antiPD-L1 when compared to PBMCs alone.

### Example 4

### Enucleated Erythroid Cells Expressing a Costimulatory Protein for Use in Treating an Infectious Disease

Approaching T-cell activation from another angle, enucleated erythroid cells were engineered to express 41-BB-L, a co-stimulatory protein that is expressed on antigen presenting cells and binds the 41-BB receptor on T-cells (enucleated erythroid cells-41-BB-L). Binding of enucleated erythroid cells-41-BB-L to recombinant 41-BB was determined using flow cytometry. Co-culture of PBMCs with enucleated erythroid cells-41-BB-L showed a 1.7 fold increase in T-cell proliferation compared to PBMCs alone. Finally, when enucleated erythroid cells-41-BB-L were incubated with Jurkat cells overexpressing 41-BB and NFkB-Luc2P, activation of NFkB-mediated luciferase expression increased 30 fold compared to controls.

### Example 5

### Generation of Enucleated Erythroid Cells Comprising 4-1BB-L and anti-PD-L1 for Use in Treating an Infectious Disease

The genes for human 4-1BB-L (SEQ ID NO: 1) and anti PD-L1 (SEQ ID NO: 2) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)) or Flag epitope tag (SEQ ID NO: 5).

Human CD34+ cells derived from mobilized peripheral blood cells from normal human donors are purchased frozen from AllCells Inc. Cells are thawed and cultured at a density of 1E5 cells/mL. Conditions for culturing CS34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then transduced with the lentivirus. Lentivirus transduction is described, e.g., in WO2015/073587, which is herein incorporated by reference in its entirety.

Transduced cells are differentiated in defined media to erythroid lineage cells and to enucleated reticulocytes, e.g., as described in WO2015/073587.

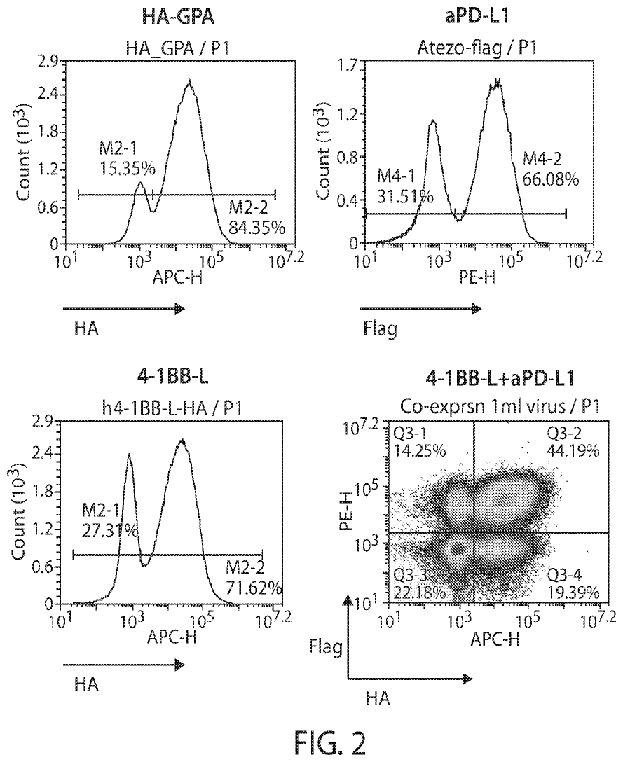

Expression of the transgenes is monitored by labeling with anti-HA and anti-Flag that had been chemically conjugated to complementary fluorescent dyes PE and APC and staining by flow cytometry. Expression levels of both ligands 4-1BB-L and anti-PD-L1 were verified through flow cytometry (FIG. 2).

### Example 6

### Activity of Enucleated Erythroid Cells Comprising 4-1BB-L and Anti-PD-L1

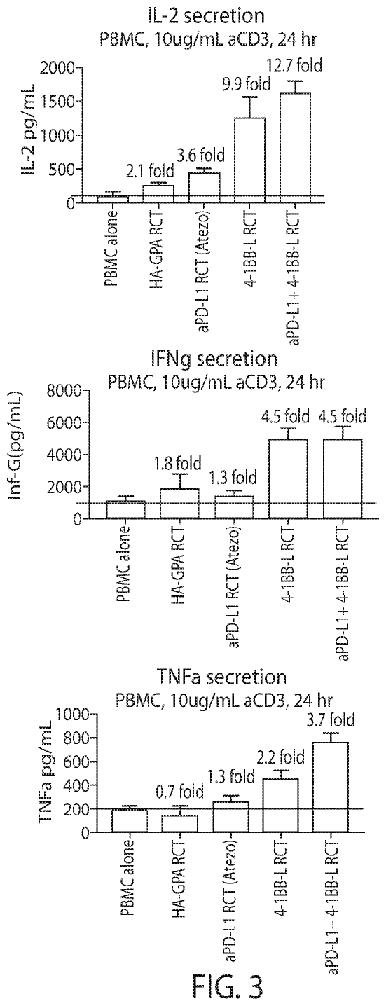

The enucleated erythroid cells expressing 4-1BB-L and anti-PD-L1 were made as described in Example 5. Peripheral blood mononuclear cells (PBMC) were purchased from Astrate bio. 200,000 PBMC were stimulated with 10 ug CD3 antibody and co-cultured with 500,000 enucleated erythroid cells for 24 hr. After 24 hr, supernatant was collected and tested for cytokine concentration using traditional ELISA for IFNg, IL-2 and TNFa. IFN-g secretion showed 1.3 and 4.5 fold increase over PBMC alone in anti-PD-L1 enucleated erythroid cells and 4-1BB-L enucleated erythroid cells, respectively, and 4.5 fold increase in the 4-1BB-L/anti-PD-L1 combination (FIG. 3). TNFa secretion showed 1.3 and 2.2 fold increase over PBMC alone in anti-PD-L1 enucleated erythroid cells and 4-1BB-L enucleated erythroid cells, respectively, and 3.7 fold increase in the 4-1BB-L/anti-PD-L1 combination.

### Example 7

### Construction of Enucleated Erythroid Cells Comprising 4-1BB-L and ICOS-L, OX40-L, and GITR-L for Use in Treating an Infectious Disease

The genes for human 4-1BB-L (SEQ ID NO: 1) and ICOS-L (SEQ ID NO: 6) or OX40-L (SEQ ID NO: 7) or GITR-L (SEQ ID NO: 8) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)).

Human CD34+ cells derived from mobilized peripheral blood cells from normal human donors are purchased frozen from AllCells Inc. Cells are thawed and cultured at a density of 1E5 cells/mL. Conditions for culturing CS34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then transduced with the lentivirus. Lentivirus transduction is described, e.g., in WO2015/073587.

Transduced cells are differentiated in defined media to erythroid lineage cells and to enucleated reticulocytes, e.g., as described in WO2015/073587.

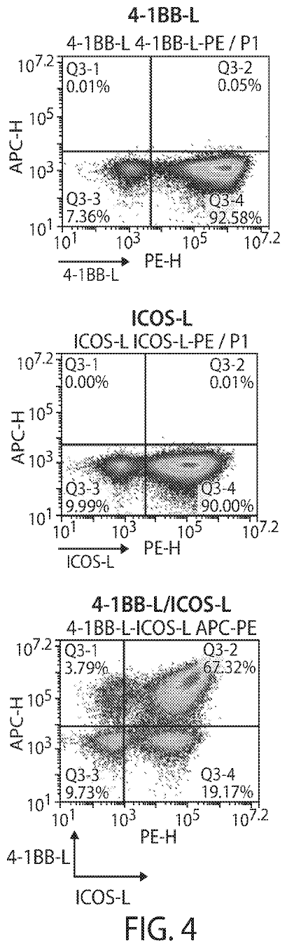

Expression of the transgenes is monitored by labeling with anti-4-1BB-L, anti-ICOS-L, anti-OX40-L or anti-GITR-L that had been chemically conjugated to complementary fluorescent dyes PE and APC and staining by flow cytometry. Expression was verified with flow cytometry. Expression levels of 4-1BB-L and ICOS-L are shown in FIG. 4.

### Example 8

### Activity of Enucleated Erythroid Cells Comprising 4-1BB-L and ICOS-L, OX40-L, and GITR-L for CD4 Cell Proliferation

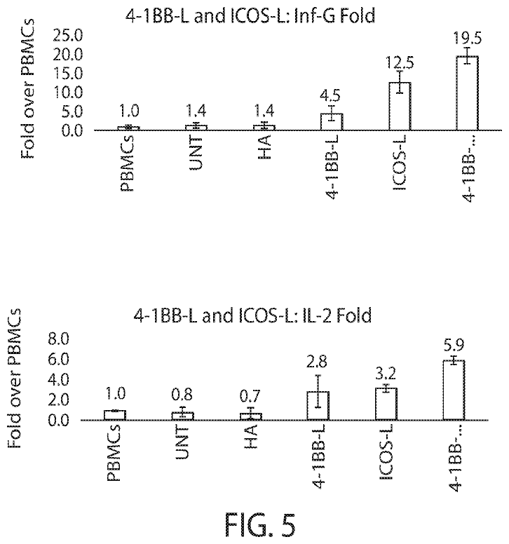

The enucleated erythroid cells comprising 4-1BB-L (SEQ ID NO: 1) and ICOS-L (SEQ ID NO: 6) were made as described in Example 7. PBMC were purchased from Astrate bio. 200,000 PBMC were stimulated with 2 ug CD3 antibody and co-cultured with 500,000 enucleated erythroid cells for 24 hr. After 24 hr, supernatant was collected and tested for cytokine concentration using traditional ELISA for IFNg and IL-2. IFN-g secretion showed 4.5 and 12.5 fold increase over PBMC alone in 4-1BB-L enucleated erythroid cells and ICOS-L enucleated erythroid cells, respectively and 19.5 fold increase in the 4-1BB-L/ICOS-L combination. IL-2 secretion showed 2.8 and 3.2 fold increase over PBMC alone in 4-1BB-L enucleated erythroid cells and ICOS-L enucleated erythroid cells, respectively, and 5.9 fold increase in the 4-1BB-L/ICOS-L combination (FIG. 5).

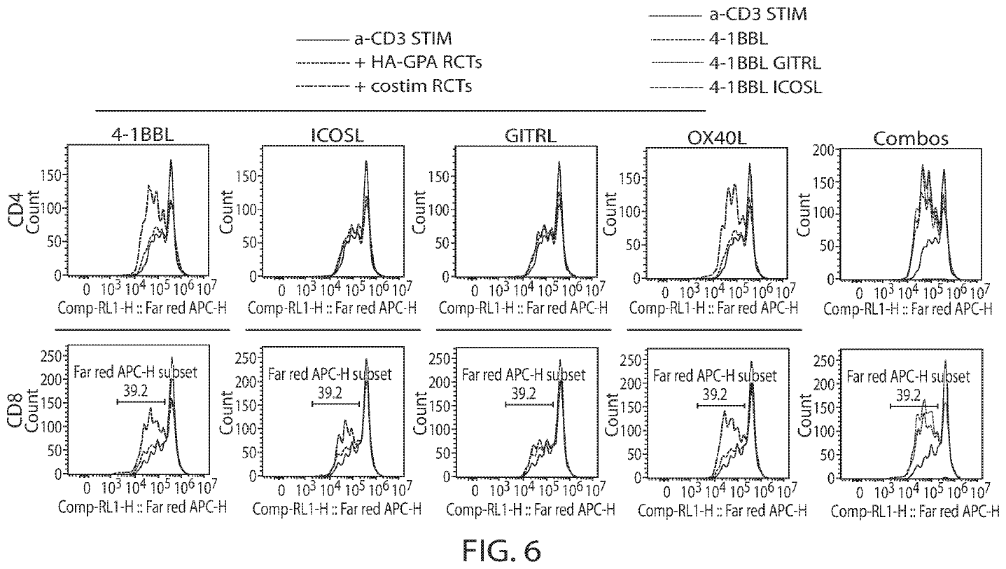

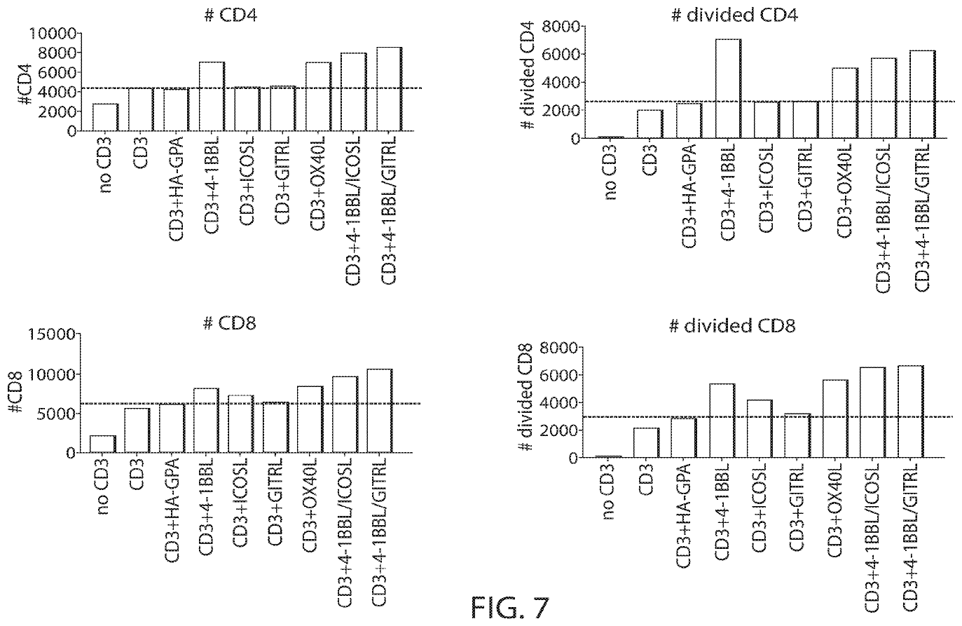

The enucleated erythroid cells expressing 4-1BB-L, ICOS-L, OX40-L (SEQ ID NO: 7), GITR-L (SEQ ID NO: 8), 4-1BB-L/ICOS-L combination, and 4-1BB-L/GITR-L combination were made as described in Example 7. PBMC were purchased from Astrate bio. 100,000 PBMC were labelled with CTFR (Thermo-Fisher), stimulated with 0.5ug/mL CD3 antibody and co-cultured with 50,000 enucleated erythroid cells for 3 days. Proliferation of PBMC was measured by assaying CTFR dilution (FIG. 6) and quantified by looking at total number of CD4 and CD8 cells, as well as the number of proliferating CD4 and CD8 cells (FIG. 7). These results demonstrate increased CD4 cell proliferation for the PBMC co-cultured with 4-1BB-L, OX40-L, and combinations of 4-1BB-L/ICOS-L and 4-1BB-L/GITR-L.

### Example 9

### Construction of Enucleated Erythroid Cells Comprising Asparaginase and Asparagine Transporter for Use In Treating Malaria

The genes for Erwinia chrysanthemi asparaginase (SEQ ID NO: 9) and an asparagine transporter such sodium-coupled neutral amino acid transporter 5 (SN2; SLC38A; SEQ ID NO: 10) or sodium-coupled neutral amino acid transporter 2 (SAT2; SLC38A2; SEQ ID NO: 11) are synthesized. The genes are cloned into a lentivirus vector (SBI).

Human CD34+ cells derived from mobilized peripheral blood cells from normal human donors are purchased frozen from AllCells Inc. Cells are thawed and cultured at a density of 1E5 cells/mL. Conditions for culturing CS34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then co-transduced with the lentivirus for asparaginase and an asparagine transporter described above. Lentivirus transduction is described, e.g., in WO2015/073587.

Transduced cells are differentiated in defined media to erythroid lineage cells and to enucleated reticulocytes, e.g., as described in WO2015/073587.

Expression of asparaginase and an asparagine transporter is monitored by Western blot analysis using antibodies specific for asparaginase and the transporter being expressed.

### Example 10

### Activity of Enucleated Erythroid Cells Comprising Asparaginase and an Asparagine Transporter In a Malaria Mouse Model

The enucleated erythroid cells comprising asparaginase and an asparagine transporter are made as described in Example 9 and prepared using sequences for Erwinia chrysanthemi asparaginase and murine SN2 or murine SAT2. The resultant enucleated erythroid cells are then formulated in a buffer appropriate for enucleated erythroid cells and dose of between le7 and 1e11 enucleated erythroid cells is administered intravenously to malaria mouse model as described in Nagaraj et al, Nat. Commun. 6: 8775, 2015; incorporated by reference herein in its entirety. Plasmodium berghei-infected mice treated with asparaginase and an asparagine transporter expressing enucleated erythroid cells are assayed for liver-stage sporozoites compared to mice untreated with enucleated erythroid cells, wherein activity is observed when the treated mice have fewer sporozoites than untreated mice. The treated mice are also assayed for the ability to produce Pb male gametocytes unable to exflagellate, compared to mice untreated with enucleated erythroid cells.

### Example 11

### Generation of Enucleated Erythroid Cells Comprising Asparaginase and Asparagine Transporter SN2 or SAT2 for Use In Treating Acute Myeloblastic Leukemia (AML) and Acute Lymphoblastic Leukemia (ALL)

The genes for Erwinia chrysanthemi asparaginase (SEQ ID NO: 9) and an asparagine transporter, such as sodium-coupled neutral amino acid transporter 5 (SN2; SLC38A; SEQ ID NO: 10) or sodium-coupled neutral amino acid transporter 2 (SAT2; SLC38A2; SEQ ID NO: 11), are synthesized. The genes are cloned into a lentivirus vector (SBI).

Human CD34+ cells derived from mobilized peripheral blood cells from normal human donors are purchased frozen from AllCells Inc. Cells are thawed and cultured at a density of 1E5 cells/mL. Conditions for culturing CS34+ cells are described, e.g., in WO2015/073587.

Virus production protocol is conducted as follows. Briefly, HEK293T cells are seeded 24 hours before transfection. Cells are transfected with lentivector containing the construct along with packaging plasmids, and viruses are harvested. Cells are then co-transduced with the lentivirus for asparaginase and an asparagine transporter described above. Lentivirus transduction is described, e.g., in WO2015/073587. Transduced cells are differentiated in defined media to erythroid lineage cells and to enucleated erythroid cells, e.g., as described in WO2015/073587.

Expression of asparaginase and an asparagine transporter is monitored by Western blot analysis using antibodies specific for asparaginase and the transporter being expressed.

### Example 12

### Activity of Enucleated Erythroid Cells Comprising Asparaginase and an Asparagine Transporter in an AML Mouse Model

The enucleated erythroid cells comprising asparaginase and an asparagine transporter is made as described in Example 11 and prepared using sequences for Erwinia chrysanthemi asparaginase and murine SN2 or murine SAT2. The resultant enucleated erythroid cells are then formulated in a buffer appropriate for erythroid cells and dose of between le7 and le 11 enucleated erythroid cells is administered intravenously to xenograph mouse model for AML as described in Kohnken et al, Front. Oncol. 2017; 7: 22, 2017; incorporated herein by reference in its entirety. In this assay, activity leads to reduction in the number of tumor cells in NOD/SCID mice transplanted with AML-derived cell lines compared to untreated mice.

### Example 13

### Generation of Enucleated Erythroid Cells Comprising CD14 and TLR2 for Use In Treating an Infectious Disease

The genes for human CD14 (SEQ ID NO: 12; Uniprot P08571) and human TLR2 (SEQ ID NO: 14; Uniprot 060603) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The enucleated erythroid cells expressing surface CD14 and TLR2 fusions are produced as described in Example 5.

### Example 14

### Activity of Enucleated Erythroid Cells Comprising CD14 and TLR2 in a Murine Infection Model

The enucleated erythroid cells comprising CD14 and TLR2 are then formulated in a buffer appropriate for enucleated erythroid cells. Administration of the enucleated erythroid cells comprising CD14 and TLR2 may result in reduce levels of infectious virus in the murine CMV model, which is used to understand human CMV disease, e.g., relative to controls, as described in Brune et al. Current protocols in immunology 19.7.1-19.7.13, 1999, and Cekinovic et al. Methods Mol Biol. 1119: 289-310, 2014; each of which is herein incorporated by reference in its entirety. Virus titers may be obtained through plaque-forming cell (PFC) assays.

### Example 15

### Construction of Enucleated Erythroid Cells Comprising CD14 and TLR4 for Use In Treating an Infectious Disease

The genes for human CD14 (SEQ ID NO: 12; Uniprot P08571) and human TLR4 (SEQ ID NO: 13; Uniprot 000206) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The enucleated erythroid cell expressing surface CD14 and TLR4 fusions are produced as described in Example 5

### Example 16

### Activity of Enucleated Erythroid Cells Comprising CD14 and TLR4 In a Murine Infection Model

The enucleated erythroid cells comprising CD14 and TLR4 are then formulated in a buffer appropriate for enucleated erythroid cells. Administration of the enucleated erythroid cells comprising CD14 and TLR4 in the murine cytomegalovirus (CMV) model, which is used to understand human CMV disease, e.g., relative to controls, may reduce levels of infectious virus as described in Brune et al. Current protocols in immunology 19.7.1-19.7.13, 1999, and Cekinovic et al. Methods Mol. Biol. 1119: 289-310, 2014; each of which is incorporated herein by reference in its entirety. Virus titers are obtained through plaque-forming cell (PFC) assays.

### Example 17

### Construction of Enucleated Erythroid Cells Comprising TLR2 and TLR4 for Use In Treating an Infectious Disease

The genes for human TLR2 (SEQ ID NO: 14; Uniprot 060603) and human TLR4 (SEQ ID NO: 13; Uniprot 000206) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The enucleated erythroid cell expressing surface CD14 and TLR4 fusions are produced as described in Example 5.

### Example 18

### Activity of Enucleated Erythroid Cells Expressing TLR2 and TLR4 In a Murine Infection Model

The enucleated erythroid cells expressing TLR2 and TLR4 is then formulated in a buffer appropriate for enucleated erythroid cells. Administration of the enucleated erythroid cells expressing TLR2 and TLR4 in the murine cytomegalovirus (CMV) model, which is used to understand human CMV disease, may reduce levels of infectious virus as described in Brune et al. Current protocols in immunology 19.7.1-19.7.13, 1999, and Cekinovic et al. Methods Mol. Biol. 1119: 289-310, 2014; each of which is incorporated herein by reference in its entirety. Virus titers are obtained through plaque-forming cell (PFC) assays.

An alternative cecal ligation and puncture (CLP) model of bacterial peritonitis can be tested as an animal model of sepsis. An active cell, in this assay, increases survival, reduces bacteria load, and/or reduces levels of inflammatory cytokines as described in Fink M, Virulence. 5(1): 143-153, 2014, Toscano et al. J. Vis. Exp. 7;(51), 2011, and Cuenca et al. Curr. Protoc. Immunol. Chapter 19: unit 19.13, 2010; each of which is incorporated herein by reference in its entirety.

### Example 19

### Construction of Enucleated Erythroid Cells Comprising CD4 and CCR5 for Use In Treating an Infectious Disease

The genes for CD4 (SEQ ID NO: 15; Uniprot P01730) and peptide variants of CCR5p18 (SEQ ID NO: 16; uniprot P51681) and CCR5p30 (SEQ ID NO: 17; P51681) are synthesized. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)). The enucleated erythroid cell expressing surface CD4 and CCR5 peptide are produced as described in Example 5. The cells were then washed with phosphate buffered saline with 0.1% bovine serum albumin, and stained with an anti-HA antibody linked to a fluorophore (phycoerythrin). The cells were then analyzed via flow cytometry to determine the protein expression. The prototype expression was determined to be in the range of 88.3-98.4% (Table 7). This experiment indicates that it is possible to express a very high percentage of the cells with two different prototypes.

TABLE 7
Expression of CD4 and CCR5 variants from enucleated
erythroid cells comprising CD4, CCR5p18, CCR5p30, CD4
and CCR5p18, and CD4 and CCR5p30.

| Prototype | Detection | Percent HA expression |
|---|---|---|
| CD4 | HA tag | 88.3% |
| CCR5p18 | HA tag | 96.9% |
| CCR5p30 | HA tag | 98.4% |
| CD4+CCR5p18 | HA tag | 95.8% |
| CD4+CCR5p30 | HA tag | 95.6% |

### Example 20

### Generation of Enucleated Erythroid Cells Comprising Anti-IgE, Anti-TNFa, Anti-PCSK9, and Anti-IL6 R for Use In Treating Bacteremia

The genes for anti-IgE (SEQ ID NO: 18), anti-TNFa (SEQ ID NO: 19), anti-PCSK9 (SEQ ID NO: 20), and anti-IL6 R (SEQ ID NO: 21) are synthesized by a commercial vendor. The genes are cloned into a lentivirus vector (SBI) upstream of the gene for human glycophorin A and separated by a sequence encoding a 12-amino acid Gly-Ser (GGGSGGGSGGGS (SEQ ID NO: 3)) flexible linker and an HA epitope tag (YPYDVPDY (SEQ ID NO: 4)).

Human CD34+ cells can be cultured, and virus can be produced, as described in Example 5. Transduced cells are differentiated as described herein.

To assess the expression of the transgenes, cells are labeled simultaneously with the ligands IgE, TNFa, PCSK9, and IL-6 (purchased from Life Technologies) and lipopolysaccharide (ThermoFisher), which are chemically conjugated to complementary fluorescent dyes. The cells are analyzed by flow cytometry to verify that (a) the agents are all expressed on the surface of the cell and (b) the agents are capable of binding to their target ligands.

The cell population is formulated in AS-3 additive solution and administered intravenously to a patient who is, e.g, developing bacteremia. Administration of the enucleated erythroid cells comprising anti-IgE, anti-TNFa, anti-PCSK9, and anti-IL6 R may result in the patient exhibiting an improvement in bacteremia and/or sepsis symptoms as measured by a reduction in circulating cytokine levels, a reduction or prevention of vascular leak syndrome, and improved survival.

### Example 21

### Capture and Modification of a Target Protein

In this Example, transgenic enucleated erythroid cells were used to capture and modify a target protein. The control cells and the experimental cells each comprise endogenous glycophorin A (GPA) in their membranes, which was used to bind the target protein. The experimental cells expressed an exogenous protein comprising surface-exposed IdeS fused to GPA as a membrane anchor. IdeS is capable of cleaving antibodies to produce a F(ab′)2 fragment and a Fc fragment. The target protein is an anti-GPA antibody that is fluorescently labelled with FITC. Both the constant and variable regions of the target antibody were FITC-labelled, so that if the antibody was cleaved, both fragments could be detected.

First, the control cells and IdeS-expressing cells were tested by FACS for the ability to bind the anti-GPA antibody. Both control and IdeS-expressing cells bound the antibody as measured by association of FITC with the cells. In addition, both control and IdeS-expressing cells bound the antibody as measured by or using a second detection method with a fluorescently labeled anti-rabbit Fc antibody. These measurements were taken at an early timepoint, before cells were incubated to allow IdeS-mediated cleavage of the target antibody.

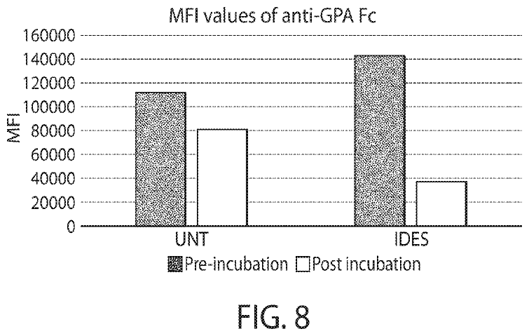

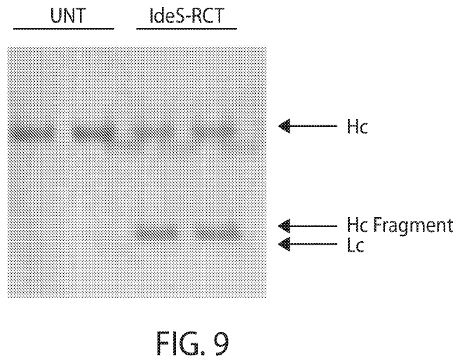

In contrast, only the IdeS-expressing cells were able to cleave the target antibody. This was shown by incubating the control or IdeS-expressing cells with the target antibody to allow antibody cleavage to occur. Fluorescently labeled anti-rabbit Fc antibody was added to the reaction in order to detect intact antibodies on the surface of the erythroid cells. The IdeS-expressing cells showed a decrease in anti-rabbit Fc association with the cells (FIG. 8), indicating lower levels of Fc on the surface of the IdeS-expressing cells compared to the control cells. There was no decrease in the amount of the directly FITC-labeled target antibody associated with control cells or IdeS-expressing cells, indicating that at least the FITC-labeled variable region of the target antibody still bound the IdeS-expressing and control cells. This result was confirmed by Western blot, where anti rabbit heavy chain and anti rabbit light chain antibodies were used to detect intact and cleaved antibody in the supernatant of control or IdeS-expressing cells. The experiment showed that IdeS-expressing erythroid cells but not control erythroid cells cleaved the anti-GPA-antibody, resulting in appearance of the heavy chain fragment (FIG. 9).

Thus, the control cells were able to bind the target antibody, but only the IdeS-expressing cells were able to bind and cleave the target antibody.

1. An enucleated erythroid cell, comprising a first exogenous polypeptide comprising a first infectious disease therapeutic, and a second exogenous polypeptide, comprising a second infectious disease therapeutic,
wherein the first and second exogenous polypeptides have agent-additive, agent-synergistic, multiplicative, independent function, localization-based, proximity-dependent, scaffold-based, multimer-based, pathway-based, or compensatory activity.

2. An enucleated erythroid cell, comprising a first exogenous polypeptide comprising a first infectious disease therapeutic, a second exogenous polypeptide, comprising a second infectious disease therapeutic, and a third exogenous polypeptide comprising a third infectious disease therapeutic.

3. An enucleated erythroid cell, comprising a first exogenous polypeptide comprising a first infectious disease therapeutic, and a second exogenous polypeptide, comprising a second infectious disease therapeutic, wherein:
a) the first and second exogenous polypeptides act on the same target, wherein optionally the target is a cell surface receptor and/or an endogenous human protein;
b) the first exogenous polypeptide binds to a first endogenous human protein and the second exogenous polypeptide binds to a second endogenous human target protein, e.g., with a Kd of less than 500, 200, 100, 50, 20, 10, 5, 2, or 1 nM;
c) the first exogenous polypeptide acts on (e.g., binds) a first target, and the second exogenous polypeptide act on (e.g., binds) a second target, wherein the first and second targets are members of the same biological pathway, wherein optionally the targets are cell surface receptors, endogenous human proteins, or both;
d) the first exogenous polypeptide comprises a first pro-apoptotic polypeptide and the second exogenous polypeptide comprises a second pro-apoptotic polypeptide, e.g., a TRAIL receptor ligand, e.g., a TRAIL polypeptide;
e) the first and second exogenous polypeptides are in close proximity to each other, e.g., are less than 10, 7, 5, 4, 3, 2, 1, 0.5, 0.2, or 0.1 nm apart for a duration of at least 1, 2, 5, 10, 30, or 60 seconds; 1, 2, 5, 10, 30, or 60 minutes, or 1, 2, 3, 6, 12, or 14 hours;
f) the first and second exogenous polypeptides have a Kd of less than 500, 200, 100, 50, 20, 10, 5, 2, or 1 nM for each other;
g) the first exogenous polypeptide comprises an antigen-presenting polypeptide, e.g., an MHC molecule, e.g., an MHC class II molecule, and the second exogenous polypeptide comprises an antigen;
h) the first and second exogenous polypeptides act on different targets, wherein optionally at least one of the targets is a cell surface receptor and/or an endogenous human protein, e.g., the first exogenous polypeptide binds a first cell type, e.g., an immune effector cell, and the second exogenous polypeptide binds a second cell type, e.g., an immune effector cell, e.g., a T cell;
i) the first exogenous polypeptide and the second exogenous polypeptide have an abundance ratio of about 1:1, from about 2:1 to 1:2, from about 5:1 to 1:5, from about 10:1 to 1:10, from about 20:1 to 1:20, from about 50:1 to 1:50, from about 100:1 to 1:100 by weight or by copy number;
j) the first exogenous polypeptide and the second exogenous polypeptide have a Kd for a first target and a second target, respectively, with a ratio of about 1:1, from about 2:1 to 1:2, from about 5:1 to 1:5, from about 10:1 to 1:10, from about 20:1 to 1:20, from about 50:1 to 1:50, from about 100:1 to 1:100;
k) the first exogenous polypeptide has a first activity (e.g., binding) towards a first target, and the second exogenous polypeptide has a second activity (e.g., binding) towards the first target, e.g., the first and second exogenous polypeptides bind a single target;
l) the first exogenous polypeptide acts on (e.g., binds) a first target and the second exogenous polypeptide acts on (e.g., binds) a second target, and the first and second targets are part of the same pathway, wherein optionally the first exogenous polypeptide acts on the first target and the second exogenous polypeptide acts on the second target simultaneously;
m) the first exogenous polypeptide acts on (e.g., binds) a first target and the second exogenous polypeptide acts on (e.g., binds) a second target, and the first and second targets are part of different pathways, wherein optionally the first and second pathways both act to promote a given cellular response;
n) the first exogenous polypeptide localizes the enucleated erythroid cell to a desired site, e.g., a human cell, and the second exogenous polypeptide has a therapeutic activity, e.g., an immunomodulation activity such as a T cell activation activity or antigen presenting activity;
o) the first exogenous polypeptide binds a first cell, e.g., a first cell type, and the second exogenous polypeptide binds a second cell, e.g., a second cell type, e.g., an immune effector cell, e.g., a T cell;
p) the first exogenous polypeptide and the second exogenous polypeptide are non-human proteins;
q) the first exogenous polypeptide and the second exogenous polypeptide are both enzymes, e.g., biosynthetic enzymes;
r) the first exogenous polypeptide promotes formation of an intermediate molecule and the second exogenous polypeptide acts on the intermediate molecule;
s) the first exogenous polypeptide and the second exogenous polypeptide act on successive steps of a pathway;
t) the erythroid cell comprises at least at least 10 copies, 100 copies, 1,000 copies, 5,000 copies 10,000 copies, 25,000 copies, 50,000 copies, or 100,000 copies of each of the first exogenous polypeptide and the second exogenous polypeptide; or
u) the copy number of the first exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the second exogenous polypeptide; or
v) the copy number of the second exogenous polypeptide is no more than 10%, 20%, 30%, 40%, 50%, 60%, 70%, 80%, or 90% greater, or no more than 2, 5, 10, 20, 50, 100, 200, 500, or 1000 times greater than the copy number of the first exogenous polypeptide.

4. The erythroid cells of any of claims 1-3 for use in a method of treating a subject having an infectious disease, wherein the erythroid cells are administered to the subject in an amount effective to treat the infectious disease.

5. The erythroid cells for use of claim 4, wherein the infectious disease is HIV, Hepatitis B, bacteremia or LPS toxicity.

6. The cell or erythroid cells for use of any of the preceding claims, wherein the first exogenous polypeptide comprises:
a virus-binding polypeptide, e.g., an antibody molecule, e.g., an scFv;
an anti-viral polypeptide, e.g., a protease, e.g., neuraminidase;
CD4 or a functional variant or fragment thereof;
CCR5 or a functional variant or fragment thereof;
TLR4, CD14, MBL, rBPI21, and LPS binding protein, or a functional variant or fragment thereof, or any combination thereof;
a viral protein (e.g., Hepatitis B surface antigen or HIV antigen);
a vaccine antigen and a polypeptide that induces activation of an APC, e.g. ICOS, CD28, CD40, 4-1BB, or OX40; or
an antibacterial protein, e.g., rBPI21.

7. The cell or erythroid cells for use of any of the preceding claims, wherein the first exogenous polypeptide comprises a virus-binding polypeptide, e.g., an antibody molecule, e.g., an scFv, and the second exogenous polypeptide comprises an anti-viral polypeptide, e.g., a protease, e.g., neuraminidase.

8. The cell or erythroid cells for use of any of the preceding claims, wherein the first exogenous polypeptide binds a target, e.g., wherein the target comprises a toxin, e.g., anthrax toxin (e.g., PA, EF, or LF, or any combination thereof), a botulinum toxins (A, B, C, D, E, F, G, or any combination thereof), ricin, saxitoxin, Staphyloccocal enterotoxin B, Tetrodotoxin, or Trichothecene mycotoxins.

9. The cell or erythroid cells for use of any of the preceding claims wherein the cell comprises a plurality of antibody molecules, which bind a plurality of common bacterial, fungal, or viral pathogens.

10. The cell or erythroid cells for use of any of the preceding claims, wherein:
the first exogenous polypeptide binds an antigen presenting cell and the second exogenous polypeptide binds a T cell;
the first exogenous polypeptide comprises an MHCII alpha chain and the second exogenous polypeptide comprises an MHCII beta chain polypeptide.

11. The cell or erythroid cells for use of any of the preceding claims, wherein the first exogenous polypeptide binds to a target more strongly than the first exogenous polypeptide binds to the second exogenous polypeptide.

12. The cell or erythroid cells for use of any of the preceding claims, wherein the first exogenous polypeptide promotes fusion of the erythroid cell with a target cell.

13. The cell or erythroid cells for use of any of the preceding claims, wherein the cell comprises at least 2 but no more than 5, 6, 7, 8, 9, or 10 different exogenous polypeptides, e.g., exogenous polypeptides that are encoded by one or more exogenous nucleic acids that are not retained by the enucleated red blood cell.

14. The cell or erythroid cells for use of any of the preceding claims, wherein the exogenous polypeptides are encoded by one or more exogenous nucleic acids that are not retained by the enucleated erythroid cell.

15. The cell or erythroid cells for use of any of the preceding claims, wherein one or more (e.g., two or three) of the first, second, and optionally third exogenous polypeptides are transmembrane polypeptides or surface-anchored polypeptides.

16. The cell or erythroid cells for use of any of the preceding claims, wherein the first exogenous polypeptide interacts with, e.g., binds, a moiety on a target cell, and the second exogenous polypeptide alters a property of the target cell, e.g., kills or activates the target cell.

17. The cell or erythroid cells for use of any of the preceding claims, wherein both the first and second exogenous polypeptides have a stoichiometric mode of action, or both have a catalytic mode of action, and both are present at a similar abundance, e.g., about 1:1 or from about 2:1 to 1:2.

18. The cell or erythroid cells for use of any of the preceding claims, wherein the first exogenous polypeptide is more abundant than the second exogenous polypeptide by at least about 10%, 20%, 30%, 50%, or a factor of 2, 3, 4, 5, 10, 20, 50, or 100 (and optionally up to 10 or 100 fold) by weight or copy number.

19. The cell or erythroid cells for use of any of the preceding claims, wherein the first polypeptide has a stoichiometric mode of action and the second polypeptide has a catalytic mode of action, and the first polypeptide is more abundant than the second polypeptide.

20. The cell or erythroid cells for use of any of the preceding claims, wherein the cell has one or more of the following characteristics:
a) an osmotic fragility of less than 50% cell lysis at 0.3%, 0.35%, 0.4%, 0.45%, or 0.5% NaCl;
b) a cell volume of about 10-200 fL or a cell diameter of between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns, or between about 10 microns and about 30 microns;
c) greater than 1%, 2%, 3%, 4%, 5%, 6%, 7%, 8%, 9% or 10% fetal hemoglobin; or at least about 20, 25, or 30 pg/cell of hemoglobin; or
d) phosphatidylserine content of the outer leaflet is less than 30%, 25%, 20%, 15%, 10%, or 5% as measured by Annexin V staining.

21. The cell or erythroid cells for use of any of the preceding claims, wherein at least one, e.g., all, of the plurality of exogenous polypeptides are glycosylated.

22. The cell or erythroid cells for use of any of the preceding claims, wherein the exogenous polypeptide or polypeptides lack a sortase transfer signature such as LPXTG.

23. The cell or erythroid cells for use of any of the preceding claims, wherein:
i) at least 50, 60, 70, 80, 90, 95, or 99% of the exogenous polypeptides, e.g., fusion proteins on the surface of the erythroid cell have an identical sequence,
ii) at least 50, 60, 70, 80, 90, 95, or 99% of the exogenous polypeptides, e.g., fusion proteins have the same transmembrane region,
iii) the first and/or second exogenous polypeptide, e.g., fusion protein does not include a full length endogenous membrane protein, e.g., comprises a segment of a full length endogenous membrane protein, which segment lacks at least 1, 2, 3, 4, 5, 10, 20, 50, 100, 200, or 500 amino acids of the full length endogenous membrane protein;
iv) at least 50, 60, 70, 80, 90, 95, or 99% of the exogenous polypeptides, e.g., fusion proteins do not differ from one another by more than 1, 2, 3, 4, 5, 10, 20, or 50 amino acids,
v) the first and/or second exogenous polypeptide lacks a sortase transfer signature,
vi) the first and/or second exogenous polypeptide comprises a moiety that is present on less than 1, 2, 3, 4, or 5 sequence distinct fusion polypeptides;
vii) the first and/or second exogenous polypeptide is present as a single fusion polypeptide;
viii) the first and/or second exogenous polypeptide, e.g., fusion protein does not contain Gly-Gly at the junction of an endogenous transmembrane protein and the moiety;
ix) the first and/or second exogenous polypeptide, e.g., fusion protein does not contain Gly-Gly, or the fusion protein does not contain Gly-Gly, or does not contain Gly-Gly in an extracellular region, does not contain Gly-Gly in an extracellular region that is within 1, 2, 3, 4, 5, 10, 20, 50, or 100 amino acids of a transmembrane segment; or a combination thereof.

24. The erythroid cell or erythroid cell for use according to any of the preceding claims, wherein the first exogenous polypeptide interacts with a target, and the second exogenous polypeptide (e.g., IdeS) modifies the target.

25. A method of making an erythroid cell according to any of the preceding claims, comprising:
a) providing an erythroid cell, and
b) contacting the erythroid cell with nucleic acid encoding the first exogenous protein and nucleic acid encoding the second exogenous protein, under conditions that allow uptake of the nucleic acid by the erythroid cell, and
c) culturing the cell under conditions that allow for expression of the first and second exogenous proteins,
thereby making an erythroid cell of any of the preceding claims.

26. The method of claim 25, wherein the nucleic acid encoding the first exogenous protein and the nucleic acid encoding the second exogenous protein are separate nucleic acids.

27. The method of claim 25, wherein the nucleic acid encoding the first exogenous protein and the nucleic acid encoding the second exogenous protein are part of the same nucleic acid molecule.

28. A plurality of erythroid cells according to any of the preceding claims, e.g., wherein the plurality comprises at least 10⁸, 10⁹, 10¹⁰, 10¹¹, or 10¹² erythroid cells according to any of the preceding claims.

29. A pharmaceutical composition comprising a cell according to any of the preceding claims, or a plurality of cells according to claim 27.

30. An enucleated erythroid cell, comprising a first exogenous polypeptide comprising a first infectious disease therapeutic, and a second exogenous polypeptide, comprising a second infectious disease therapeutic, wherein:
the first infectious disease therapeutic comprises a CD14 polypeptide and the second infectious disease therapeutic comprises a TLR2 polypeptide;
the first infectious disease therapeutic comprises a TLR2 polypeptide and the second infectious disease therapeutic comprises a TLR4 polypeptide;
the first infectious disease therapeutic comprises a CD4 polypeptide and the second infectious disease therapeutic comprises a CCR5 polypeptide;
the first infectious disease therapeutic comprises a 4-1BB-L polypeptide and the second infectious disease therapeutic comprises an anti PD-L1 polypeptide;
the first infectious disease therapeutic comprises an 1BB-L polypeptide and the second infectious disease therapeutic comprises an ICOS-L polypeptide, an OX40-L polypeptide, or a GITR-L polypeptide; or
the first infectious disease therapeutic comprises an asparaginase polypeptide and the second infectious disease therapeutic comprises an asparagine transporter SN2 or SAT2 polypeptide.

31. The erythroid cells of claim 30 for use in a method of treating a subject having an infectious disease (e.g., CMV, HIV, or malaria), wherein the erythroid cells are administered to the subject in an amount effective to treat the infectious disease (e.g., CMV, HIV, or malaria).
